(19) United States
(12) Patent Application Publication
(10) Pub. No.: US 2005/0010048 A1
(43) Pub. Date: Jan. 13, 2005

(54) Aza-and polyaza-naphthalenly ketones useful as hiv integrase inhibitors

(76) Inventors: Linghang Zhuang, Chalfont, PA (US); John S. Wai, Harleysville, PA (US); Linda S. Payne, Lansdale, PA (US); Steven D. Young, Lansdale, PA (US); Thorsten E. Fisher, Hatfield, PA (US); Mark W. Embrey, North Wales, PA (US); James P.. Guare, Quakertown, PA (US)

Correspondence Address: MERCK AND CO INC, P O BOX 2000, RAHWAY, NJ, 070650907, US

(21) Appl. No.: 10/398,929

(22) PCT Filed: Oct. 9, 2001
(86) PCT No.: PCT/US01/42553

Related U.S. Application Data
(60) Provisional application No. 60/239,732, filed on Oct. 12, 2000.

(51) Int. Cl. C07D 471/02; C07D 487/02

(52) U.S. Cl. 544/280; 546/123; 544/183; 544/350

(57) ABSTRACT

Certain aza- and polyaza-naphthalenyl ketones including certain quinolinyl and naphthyridinyl ketones are described as inhibitors of HIV integrase and inhibitors of HIV replication. These compounds are useful in the prevention or treatment of infection by HIV and the treatment or the delay in the onset of AIDS, as compounds or pharmaceutically acceptable salts, or as ingredients in pharmaceutical compositions, optionally in combination with other antivirals, immunomodulators, antibiotics or vaccines. Methods of treating or delaying the onset of AIDS and methods of preventing or treating infection by HIV are also described.

### FIELD OF THE INVENTION

The present invention is directed to aza- and polyaza-naphthalenyl ketones and pharmaceutically acceptable salts thereof, their synthesis, and their use as inhibitors of the HIV integrase enzyme. The compounds of the present invention include 1-aryl-1-(poly)azanaphthylenyl methanones and 1-heterocyclyl-1-(poly)azanaphthylenyl methanones. Suitable (poly)azanapthalenyl groups include quinolinyl, naphthyridinyl, and quinoxalinyl. The compounds and pharmaceutically acceptable salts thereof of the present invention are useful for preventing or treating infection by HIV and for treating AIDS.

References are made throughout this application to various publications in order to more fully describe the state of the art to which this invention pertains. The disclosures of these references are hereby incorporated by reference in their entireties.

### BACKGROUND OF THE INVENTION

A retrovirus designated human immunodeficiency virus (HIV) is the etiological agent of the complex disease that includes progressive destruction of the immune system (acquired immune deficiency syndrome; AIDS) and degeneration of the central and peripheral nervous system. This virus was previously known as LAV, HTLV-III, or ARV. A common feature of retrovirus replication is the insertion by virally-encoded integrase of proviral DNA into the host cell genome, a required step in HIV replication in human T-lymphoid and monocytoid cells. Integration is believed to be mediated by integrase in three steps: assembly of a stable nucleoprotein complex with viral DNA sequences; cleavage of two nucleotides from the 3′ termini of the linear proviral DNA; covalent joining of the recessed 3′ OH termini of the proviral DNA at a staggered cut made at the host target site. The fourth step in the process, repair synthesis of the resultant gap, may be accomplished by cellular enzymes.

Nucleotide sequencing of HIV shows the presence of a pol gene in one open reading frame [Ratner, L. et al., Nature, 313, 277(1985)]. Amino acid sequence homology provides evidence that the pol sequence encodes reverse transcriptase, integrase and an HIV protease [Toh, H. et al., EMBO J.4, 1267 (1985); Power, M. D. et al., Science, 231, 1567 (1986); Pearl, L. H. et al., Nature, 329, 351 (1987)]. All three enzymes have been shown to be essential for the replication of HIV.

It is known that some antiviral compounds which act as inhibitors of HIV replication are effective agents in the treatment of AIDS and similar diseases, including reverse transcriptase inhibitors such as azidothymidine (AZT) and efavirenz and protease inhbitors such as indinavir and nelfinavir. The compounds of this invention are inhibitors of HIV integrase and inhibitors of HIV replication. The inhibition of integrase in vitro and HIV replication in cells is a direct result of inhibiting the strand transfer reaction catalyzed by the recombinant integrase in vitro in HIV infected cells. The particular advantage of the present invention is highly specific inhibition of HIV integrase and HIV replication.

The following references are of interest as background:

Matsumura, J. Am. Chem. Soc. 1935, 57: 124-128 discloses 7-o-carboxylic-benzoyl-8-hydroxyquinoline and its methyl ester.

Blanco et al., J. Heterocycl. Chem. 1966, 33 361-366 discloses a tautomer of 5,8-dihydroxy-7-benzoyl-1,6-naphthyridine.

Sharma et al., Monatsch. Chemie 1985, 116: 353-356 discloses 7-benzoyl-8-hydroxyquinoline.

U.S. Pat. No. 3,113,135 discloses certain 7-benzoyl-8-hydroxyquinolines and 7-benzoyl-8-hydroxyquinaldines having anti-microbial activity.

U.S. Pat. No. 5,798,365 discloses certain 4-alkylene substituted-3,4-dihydroquinoline derivatives exhibiting antiviral activity, in particular against HIV.

U.S. Pat. No. 5,324,839 and U.S. Pat. No. 5,478,938 disclose nitrogenous bicyclic derivatives substituted with benzyl having antagonistic properties for angiotensin II receptors.

U.S. Pat. No. 5,602,146 discloses 4-iminoquinolines having antiviral activity.

WO 97/37977 discloses certain 4-carbonyl and 4-carboxylic quinoline derivatives and their tautomers which are useful in treating retroviral infection such as AIDS.

### SUMMARY OF THE INVENTION

The present invention is directed to novel aza- and polyazanaphthalenyl ketones. These compounds are useful in the inhibition of HIV integrase, the prevention of infection by HIV, the treatment of infection by HIV and in the treatment of AIDS and/or ARC, either as compounds, pharmaceutically acceptable salts or hydrates (when appropriate), pharmaceutical composition ingredients, whether or not in combination with other HIV/AIDS antivirals, anti-infectives, immunomodulators, antibiotics or vaccines. More particularly, the present invention includes a compound of Formula (I): 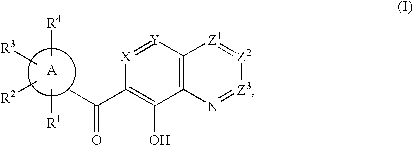
wherein A is

(1) phenyl,

(2) phenyl fused to a carbocycle to form a fused carbocyclic ring system; or

(3) heterocycle containing one or more heteroatoms selected from nitrogen, oxygen and sulfur and a balance of carbon atoms, with at least one of the ring atoms being carbon;

A is connected by a ring carbon to the exocyclic carbonyl, and is substituted by R¹, R², R³, and R⁴;

X is N or C-Q¹;
Y is N or C-Q², provided that X and Y are not both N;
Z¹ is N or C-Q³;
Z² is N or C-Q⁴;
Z³ is N or CH;
   each of Q¹, Q², Q³, and Q⁴ is independently

(1) —H,

(2) —C₁₋₆ alkyl,

(3) —C₁₋₆ fluoroalkyl,

(4) —OH,

(5) —O—C₁₋₆ alkyl,

(6) —O—C₁₋₆ fluoroalkyl,

(7) halo,

(8) —CN,

(9) —C₁₋₆ alkyl-OR^{a},

(10) —C₀₋₆ alkyl-C(═O)R^{a},

(11) —C_{O-6} alkyl-CO₂R^{a},

(12) —C₀₋₆ alkyl-SR^{a},

(13) —N(R^{a})₂,

(14) —C₁₋₆ alkyl-N(R^{a})₂,

(15) —C₀₋₆ alkyl-C(═O)N(R^{a})₂,

(16) —C₁₋₆ alkyl-N(R^{a})—C(R^{a})═O,

(17) —SO₂R^{a},

(18) —N(R^{a})SO₂R^{a},

(19) —C₂₋₅ alkynyl,

(20) —C₂₋₅ alkynyl-CH₂N(R^{a})₂,

(21) —C₂₋₅ alkynyl-CH₂OR^{a},

(22)

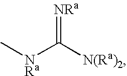

(23) —N(R^{a})—C₁₋₆ alkyl-SR^{a},

(24) —N(R^{a})—C₁₋₆ alkyl-OR^{a},

(25) —N(R^{a})—C₁₋₆ alkyl-N(R^{a})₂,

(26) —N(R^{a})—C₁₋₆ alkyl-N(R^{a})—C(R^{a})═O,

(27) —R^{k},

(28) —C₁₋₆ alkyl substituted with R^{k},

(29) —C₁₋₆ fluoroalkyl substituted with R^{k},

(30) —C₂₋₅ alkenyl-R^{k},

(31) —C₂₋₅ alkynyl-R^{k},

(32) —O—R^{k},

(33) —O—C₁₋₄ alkyl-R^{k},

(34) —S(O)ₙ—R^{k},

(35) —S(O)ₙ—C₁₋₄ alkyl-R^{k},

(36) —O—C₁₋₆ alkyl-OR^{k},

(37) —O—C₁₋₆ alkyl-O—C₁₋₄ alkyl-R^{k},

(38) —O—C₁₋₆ alkyl-SR^{k},

(39) —N(R^{c})—R^{k},

(40) —N(R^{c})—C₁₋₆ alkyl substituted with one or two R^{k} groups;

(41) —N(R^{c})—C₁₋₆ alkyl-OR^{k},

(42) —C(═O)N—C₁₋₆ alkyl-R^{k}, or

(43) —C₂₋₅ alkynyl-CH₂S(O)ₙ—R^{a};

each of R¹ and R² is independently:

(1) —H,

(2) —C₁₋₆ alkyl,

(3) —C₁₋₆ fluoroalkyl,

(4) —O—C₁₋₆ alkyl,

(5) —O—C₁₋₆ fluoroalkyl,

(6) —OH,

(7) halo,

(8) —NO₂,

(9) —CN,

(10) —C₁₋₆ alkyl-OR^{a},

(11) —CO₁₋₆ alkyl-C(═O)R^{a},

(12) —C₀₋₆ alkylCO₂R^{a},

(13) —C₀₋₆ alkyl-SR^{a},

(14) —N(R^{a})₂,

(15) —C₁₋₆ alkyl-N(R^{a})₂,

(16) —C₀₋₆ alkyl-C(═O)N(R^{a})₂,

(17) —C₁₋₆ alkyl-N(R^{a})—C(R^{a})═O,

(18) —SO₂R^{a},

(19) —N(R^{a})SO₂R^{a},

(20) —C₂₋₅ alkenyl,

(21) —O—C₁₋₆ alkyl-OR^{a},

(22) —O—C₁₋₆ alkyl-SR^{a},

(23) —O—C₁₋₆ alkyl-NH—CO₂R^{a},

(24) —O—C₂₋₆ alkyl-N(R^{a})₂,

(25) —N(R^{a})—C₁₋₆ alkyl-SR^{a},

(26) —N(R^{a})—C₁₋₆ alkyl-OR^{a},

(27) —N(R^{a})—C₁₋₆ alkyl-N(R^{a})₂,

(28) —N(R^{a})—C₁₋₆ alkyl-N(R^{a})—C(R^{a})═O,

(29) R^{k},

(30) —C₁₋₆ alkyl substituted with 1 or 2 R^{k} groups,

(31) —C₁₋₆ fluoroalkyl substituted with 1 or 2 R^{k} groups,

(32) —C₂₋₅ alkenyl-R^{k},

(33) —C₂₋₅ alkynyl-R^{k},

(34) —OR^{k},

(35) —O—C₁₋₄ alkyl-R^{k},

(36) —S(O)ₙ-R^{k},

(37) —S(O)ₙ—C₁₋₄ alkyl-R^{k},

(38) —O—C₁₋₆ alkyl-OR^{k},

(39) —O—C₁₋₆ alkyl-O—C₁₋₄ alkyl-R^{k},

(40) —O—C₁₋₆ alkyl-SR^{k},

(41) —C₁₋₆ alkyl (OR^{b})(R^{k}),

(42) —C₁₋₆ alkyl (OR^{b})(—C₁₋₄ alkyl-R^{k}),

(43) —C₀₋₆ alkyl-N(R^{b})(R^{k}),

(44) —C₀₋₆ alkyl-N(R^{b})(—C₁₋₄ alkyl-R^{k}),

(45) —C₁₋₆ alkyl S(O)ₙ-R^{k},

(46) —C₁₋₆ alkyl S(O)ₙ—C₁₋₄ alkyl-R^{k},

(47) —C₀₋₆ alkyl C(O)—R^{k}, or

(48) —C₀₋₆ alkyl C(O)—C₁₋₄ alkyl-R^{k};

each of R³ and R⁴ is independently

(1) —H,

(2) halo,

(3) —CN,

(4) —NO₂,

(5) —OH,

(6) C₁₋₆ alkyl,

(7) C₁₋₆ fluoroalkyl,

(8) —O—C₁₋₆ alkyl,

(9) —O—C₁₋₆ fluoroalkyl,

(10) —C₁₋₆ alkyl-OR^{a},

(11) —C₀₋₆ alkyl-C(═O)R^{a},

(12) —C₀₋₆ alkyl-CO₂R^{a},

(13) —C₀₋₆ alkyl-SR^{a},

(14) —N(R^{a})₂,

(15) —C₁₋₆ alkyl-N(R^{a})₂,

(16) —C₀₋₆ alkyl-C(═O)N(R^{a})₂,

(17) —SO₂R^{a},

(18) —N(R^{a})SO₂R^{a},

(19) —C₂₋₅ alkenyl,

(20) —O-C₁₋₆ alkyl-OR^{a},

(21) —O—C₁₋₆ alkyl-SR^{a},

(22) —O—C₁₋₆ alkyl-NH—CO₂R^{a},

(23) —O—C₂₋₆ alkyl-N(R^{a})₂, or

(24) oxo;

each R^{a} is independently —H, —C₁₋₆ alkyl, or —C₁₋₆ fluoroalkyl;

each R^{b} is independently:

(1) —H,

(2) —C₁₋₄ alkyl,

(3) —C₁₋₄ fluoroalkyl,

(4) R^{k},

(5) —C₂₋₃ alkenyl,

(6) —C₁₋₄ alkyl-R^{k},

(7) —C₂₋₃ alkenyl-R^{k},

(8) —S(O)ₙ—R^{k}, or

(9) —C(O)—R^{k};

each R^{c} is independently

(1) —H,

(2) —C₁₋₆ alkyl,

(3) —C₁₋₆ alkyl substituted with —N(R^{a})₂, or

(4) —C₁₋₄ alkyl-aryl, wherein aryl is optionally substituted with 1 to 5 substituents independently selected from halogen, C₁₋₆ alkyl, C₁₋₆ fluoroalkyl, —O—C₁₋₆ alkyl, —O—C₁₋₆ fluoroalkyl, —S—C₁₋₆ alkyl, —CN, and —OH;

each R^{k} is independently carbocycle or heterocycle, wherein either the carbocycle or heterocycle is unsubstituted or substituted with from 1 to 5 substituents each of which is independently selected from

(a) halogen,

(b) C₁₋₆ alkyl,

(c) C₁₋₆ fluoroalkyl,

(d) —O—C₁₋₆ alkyl,

(e) —O-C₁₋₆ fluoroalkyl,

(f) —S-C₁₋₆ alkyl,

(g) —CN,

(h) —OH,

(i) oxo,

(j) —(CH₂)₀₋₃C(═O)N(R^{a})₂,

(k) —(CH₂)₀₋₃C(═O)R^{a},

(l) —N(R^{a})—C(═O)R^{a},

(m) —N(R^{a})—C(═O)OR^{a},

(n) —(CH₂)₁₋₃N(R^{a})—C(═O)R^{a},

(o) —N(R^{a})₂,

(p) —C16 alkyl-N(R^{a})₂,

(q) aryl,

(r) aryloxy-,

(s) —C₁₋₄ alkyl substituted with aryl,

(t) heteromonocycle,

(u) —C₁₋₄ alkyl substituted with a heteromonocycle,

(v) heteromonocyclylcarbonyl-C₀₋₆ alkyl-,

(w) N-heteromonocyclyl-N-C₁₋₆ alkyl-amino-;
wherein the aryl group in (q) aryl, (r) aryloxy, and (s) —C₁₋₄ alkyl substituted with aryl, is optionally substituted with from 1 to 3 substituents independently selected from halogen, C₁₋₆ alkyl, —O—C₁₋₆ alkyl, C₁₋₆ alkyl substituted with N(R^{a})₂, C₁₋₆ fluoroalkyl, and —OH; and
wherein the heteromonocyclyl group in (t) heteromonocycle, (u) —C₁₋₄ alkyl substituted with a heteromonocycle, (v) heteromonocyclyl-carbonyl-C₀₋₆ alkyl-, and (w) N-heteromonocyclyl-N-C₁₋₆ alkyl-amino- is optionally substituted with from 1 to 3 substituents independently selected from halogen, C₁₋₆ alkyl, —O—C₁₋₆ alkyl, C₁₋₆ fluoroalkyl, oxo, and —OH; and
   each n is independently an integer equal to 0, 1 or 2;
   and provided that:

(i) when A is phenyl, X is CH, Y is CH, and Z¹=Z²=Z³=CH, then at least one of R¹, R², R³, and R⁴ is not —H;

(ii) when A is phenyl, X is CH, Y is CQ² wherein Q² is halo or —C₁₋₆ alkyl or phenyl optionally substituted with halo or —C₁₋₆ alkyl or benzyl optionally substituted with halo or —C₁₋₆ alkyl, Z¹=Z²=Z³=CH, and all but one of R¹, R², R³ and R⁴ are independently —H, halo or —C₁₋₆ alkyl, then the other of R¹, R², R³ and R⁴ is not —H, halo or —C₁₋₆ alkyl;

(iii) when A is phenyl, X is CH, Y is CH, Z¹=Z²=Z³=CH, and one of R¹, R², R³, and R⁴ is —CO₂R^{a}, then at least one of the others of R¹, R², R³, and R⁴ is not —H;

(iv) when A is phenyl, X is N, Y is C—OH, and Z¹=Z²=Z³=CH, then at least one of R¹, R², R³, and R⁴ is not —H; and

(v) when A is phenyl, X is CH, Y is CH, Z¹ is CQ³, and Z²=Z³=CH, then either (v-a) Q³ is not unsubstituted or substituted benzyl or (v-b) at least one of R¹, R², R³, and R⁴ is not —H;

or a pharmaceutically acceptable salt thereof.

The present invention also includes pharmaceutical compositions containing a compound as described above and methods of preparing such pharmaceutical compositions. The present invention further includes methods of treating AIDS, methods of delaying the onset of AIDS, methods of preventing AIDS, methods of preventing infection by HIV, and methods of treating infection by HIV.

Other embodiments, aspects and features of the present invention are either further described in or will be apparent from the ensuing description, examples and appended claims.

### DETAILED DESCRIPTION OF THE INVENTION

The present invention includes the aza- and polyaza-naphthalenyl ketones of Formula (I) above. These compounds and pharmaceutically acceptable salts thereof are HIV integrase inhibitors.

A first embodiment of the invention is a compound of Formula I,

wherein

each R^{k} is independently:

(1) aryl selected from phenyl and naphthyl, wherein aryl is unsubstituted or substituted with from 1 to 5 substituents independently selected from:
(a) halogen,
(b) C₁₋₆ alkyl,
(c) C₁₋₆ fluoroalkyl,
(d) —O—C₁₋₆ alkyl,
(e) —O—C₁₋₆ fluoroalkyl,
(f) phenyl,
(g) —S—C₁₋₆ alkyl,
(h) —CN,
(i) —OH,
(j) phenyloxy, unsubstituted or substituted with from 1 to 3 substituents independently selected from:
   (i) halogen,
   (ii) C₁₋₆ alkyl,
   (iii) C₁₋₆ fluoroalkyl, and
   (iv) —OH,
(k) —N(R^{a})₂,
(l) —C₁₋₆ alkyl-N(R^{a})₂,
(m) —R^{t},
(p) —(CH₂)₀₋₃C(═O)N(R^{a})₂, and
(q) —(CH₂)₀₋₃C(═O)R^{a};

(2) —C₃₋₇ cycloalkyl, unsubstituted or substituted with from 1 to 3 substituents independently selected from:
(a) halogen,
(b) C₁₋₆ alkyl,
(c) —O-C₁₋₆ alkyl,
(d) C₁₋₆ fluoroalkyl,
(e) —O—C₁₋₆ fluoroalkyl,
(f) —CN,
(h) phenyl, and
(j) —OH;

(3) —C₃₋₇ cycloalkyl fused with a phenyl ring, unsubstituted or substituted with from 1 to 5 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,
(f) —CN, and
(g) —OH;

(4) a 5- or 6-membered heteroaromatic ring containing from 1 to 4 heteroatoms independently selected from oxygen, nitrogen and sulfur, wherein the heteroaromatic ring is unsubstituted or substituted on nitrogen or carbon with from 1 to 5 substituents independently selected from:
(a) halogen,
(b) C₁₋₆ alkyl,
(c) C₁₋₆ fluoroalkyl,
(d) —O—C₁₋₆ alkyl,
(e) —O—C₁₋₆ fluoroalkyl,
(f) phenyl,
(g) —S—C₁₋₆ alkyl,
(h) —CN,
(i) —OH,
(j) phenyloxy, unsubstituted or substituted with from 1 to 3 substituents independently selected from:
   (i) halogen,
   (ii) C₁₋₆ alkyl,
   (iii) C₁₋₆ fluoroalkyl, and
   (iv) —OH,
(k) —N(R^{a})₂,
(l) —C₁₋₆ alkyl-N(R^{a})₂,
(m) —R^{t},
(n) oxo,
(o) —(CH₂)₀₋₃C(═O)N(R^{a})₂, and
(p) —(CH₂)₀₋₃C(═O)R^{a};

(5) a 5- or 6-membered saturated heterocyclic ring containing 1 or 2 heteroatoms independently selected from oxygen, nitrogen and sulfur, wherein the heterocyclic ring is unsubstituted or substituted with from 1 to 4 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,
(f) —CN,
(g) oxo,
(h) phenyl
(i) benzyl,
(j) phenylethyl,
(k) —OH,
(l) —(CH₂)₀₋₃C(═O)N(R^{a})₂,
(m) —(CH₂)₀₋₃C(═O)R^{a},
(n) —N(R^{a})—C(═O)R^{a},
(o) —N(R^{a})—C(═O)OR^{a},
(p) —(CH₂)₁₋₃N(R^{a})—C(═O)R^{a},
(q) —N(R^{a})₂,
(r) —(CH₂)₁₋₃N(R^{a})₂,
(s) —(CH₂)₀₋₃C(═O)R^{t},
(t) —R^{t},
(u) —N(R^{a})R^{t}, and
(v) —(CH₂)₁₋₃R^{t}; or

(6) an 8- to 10- membered heterobicyclic ring containing from 1 to 4 heteroatoms independently selected from oxygen, nitrogen and sulfur, wherein the heterobicyclic ring is saturated or unsaturated and is unsubstituted or substituted with from 1 to 5 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,
(f) —CN,
(g) ═O, and
(h) —OH; and
   R^{t} is naphthyl or a 5- or 6-membered heteromonocylic ring containing from 1 to 4 nitrogen atoms, wherein the heteromonocyclic ring is saturated or unsaturated, and wherein the naphthyl or the heteromonocyclic ring is unsubstituted or substituted with 1 or 2 substituents independently selected from halogen, oxo, C₁₋₄ alkyl, and —O—C₁₋₄ alkyl;
   and all other variables are as originally defined above;
   and provided that:

(i) when A is phenyl, X is CH, Y is CH, and Z¹=Z²=Z³=CH, then at least one of R¹, R², R³, and R⁴ is not —H;

(ii) when A is phenyl, X is CH, Y is CQ² wherein Q² is halo or —C₁₋₆ alkyl or phenyl optionally substituted with halo or —C₁₋₆ alkyl or benzyl optionally substituted with halo or —C₁₋₆ alkyl, Z¹=Z²=Z³=CH, and all but one of R¹, R², R³ and R⁴ are independently —H, halo or —C₁₋₆ alkyl, then the other of R¹, R², R³ and R⁴ is not —H, halo or —C₁₋₆ alkyl;

(iii) when A is phenyl, X is CH, Y is CH, Z¹=Z²=Z³=CH, and one of R¹, R², R³, and R⁴ is —CO₂R^{a}, then at least one of the others of R¹, R², R³, and R⁴ is not —H;

(iv) when A is phenyl, X is N, Y is C—OH, and Z¹=Z²=Z³=CH, then at least one of R¹, R², R³, and R⁴ is not —H; and

(v) when A is phenyl, X is CH, Y is CH, Z¹ is CQ³, and Z²=Z³=CH, then either (v-a) Q³ is not unsubstituted or substituted benzyl or (v-b) at least one of R¹, R², R³, and R⁴ is not —H;

or a pharmaceutically acceptable salt thereof.

A second embodiment of the invention is a compound of Formula (I),

wherein

each R^{k} is independently:

(1) aryl selected from phenyl and naphthyl, wherein aryl is unsubstituted or substituted with from 1 to 4 substituents independently selected from:
(a) halogen,
(b) C₁₋₆ alkyl,
(c) C₁₋₆ fluoroalkyl,
(d) —O—C₁₋₆ alkyl,
(e) —O—C₁₋₆ fluoroalkyl,
(f) phenyl,
(g) —S—C₁₋₆ alkyl,
(h) —CN,
(i) —OH,
(j) phenyloxy, unsubstituted or substituted with from 1 to 3 substituents independently selected from:
   (i) halogen,
   (ii) C₁-₆ alkyl,
   (iii) C₁₋₆ fluoroalkyl, and
   (iv) —OH,
(k) —N(R^{a})₂,
(l) —C₁₋₆ alkyl-N(R^{a})₂,
(m) R^{t},
(p) —(CH₂)₀₋₃C(═O)N(R^{a})₂, and
(q) —(CH₂)₀₋₃C(═O)R^{a};

(2) —C₃₋₆ cycloalkyl, unsubstituted or substituted with from 1 to 3 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,
(f) —CN,
(h) phenyl, and
(j) —OH;

(3) —C₃₋₆ cycloalkyl fused with a phenyl ring, unsubstituted or substituted with from 1 to 4 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,
(f) —CN, and
(g) —OH;

(4) a 5- or 6- membered heteroaromatic ring selected from thienyl, pyridyl, imidazolyl, pyrrolyl, pyrazolyl, thiazolyl, isothiazolyl, oxazolyl, isooxazolyl, pyrazinyl, pyrimidinyl, triazolyl, tetrazolyl, furanyl, and pyridazinyl, wherein the heteroaromatic ring is unsubstituted or substituted on nitrogen or carbon with from 1 to 4 substituents independently selected from:
(a) halogen,
(b) C₁₋₆ alkyl,
(c) C₁₋₆ fluoroalkyl,
(d) —O—C₁₋₆ alkyl,
(e) —O—C₁₋₆ fluoroalkyl,
(f) phenyl,
(g) —S—C₁₋₆ alkyl,
(h) —CN,
(i) —OH,
(j) phenyloxy, unsubstituted or substituted with from 1 to 3 substituents independently selected from:
   (i) halogen,
   (ii) C₁₋₆ alkyl,
   (iii) C₁₋₆ fluoroalkyl, and
   (iv) —OH,
(k) —N(R^{a})₂,
(l) —C₁₋₆ alkyl-N(R^{a})₂,
(m) R^{t},
(n) oxo,
(o) —(CH₂)₀₋₃C(═O)N(R^{a})₂, and
(p) —(CH₂)₀₋₃C(═O)R^{a};

(5) a 5- or 6- membered saturated heterocyclic ring selected from piperidinyl, morpholinyl, thiomorpholinyl, thiazolidinyl, isothiazolidinyl, oxazolidinyl, isooxazolidinyl, pyrrolidinyl, imidazolidinyl, piperazinyl, tetrahydrofuranyl, and pyrazolidinyl, wherein the heterocyclic ring is unsubstituted or substituted with from 1 to 3 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,
(f) —CN,
(g) ═O,
(h) phenyl
(i) benzyl,
(j) phenylethyl,
(k) —OH,
(l) —(CH₂)₀₋₃C(═O)N(R^{a})₂,
(m) —(CH₂)₀₋₃C(═O)R^{a},
(n) N(R^{a})—C(═O)R^{a},
(o) N(R^{a})—C(═O)OR^{a},
(p) (CH₂)₁₋₃N(R^{a})—C(═O)R^{a},
(q) N(R^{a})₂,
(r) (CH₂)₁₋₃N(R^{a})₂,
(s) —(CH₂)₀₋₃C(═O)R^{t},
(t) —R^{t},
(u) —N(R^{a})R^{t}, and
(v) —(CH₂)₁₋₃R^{t}; or

(6) an 8- to 10- membered heterobicyclic ring selected from indolyl, benzotriazolyl, benzoimidazolyl, imidazo[4,5-b]pyridinyl, dihydroimidazo[4,5-b]pyridinyl, pyrazolo[4,3-c]pyridinyl, dihydropyrazolo[4,3-c]pyridinyl, tetrahydropyrazolo[4,3-c]pyridinyl, pyrrolo[1,2-a]pyrazinyl, dihydropyrrolo[1,2-a]pyrazinyl, tetrahydropyrrolo[1,2-a]pyrazinyl, octahydropyrrolo[1,2-a]pyrazinyl, isoindolyl, indazolyl, indolinyl, isoindolinyl, quinolinyl, isoquinolinyl, quinoxalinyl, quinazolinyl, cinnolinyl, chromanyl, and isochromanyl, wherein the bicyclic ring is unsubstituted or substituted with 1 or 2 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,
(f) —CN,
(g) ═O, and
(h) —OH; and
   R^{t} is naphthyl or a 5- or 6-membered heteromonocylic ring selected from pyrrolidinyl, pyrazolidinyl, imidazolinyl, piperidinyl, piperazinyl, pyrrolyl, pyridyl, imidazolyl, pyrazolyl, triazolyl, tetrazolyl, pyrazinyl, pyrimidinyl, and pyradizinyl; and wherein the naphthyl or the heteromonocyclic ring is unsubstituted or substituted with 1 or 2 substituents independently selected from halogen, oxo, C₁₋₄ alkyl, and —O—C₁₋₄ alkyl;
   and all other variables are as originally defined above;
   and provided that:

(i) when A is phenyl, X is CH, Y is CH, and Z¹=Z²=Z³=CH, then at least one of R¹, R², R³, and R⁴ is not —H;

(ii) when A is phenyl, X is CH, Y is CQ² wherein Q² is halo or —C₁₋₆ alkyl or phenyl optionally substituted with halo or —C₁₋₆ alkyl or benzyl optionally substituted with halo or —C₁₋₆ alkyl, Z¹=Z²=Z³=CH, and all but one of R¹, R², R³ and R⁴ are independently —H, halo or —C₁₋₆ alkyl, then the other of R¹, R², R³ and R⁴ is not —H, halo or —C₁₋₆ alkyl;

(iii) when A is phenyl, X is CH, Y is CH, Z¹=Z²=Z³=CH, and one of R¹, R², R³, and R⁴ is —CO₂R^{a}, then at least one of the others of R¹, R², R³, and R⁴ is not —H;

(iv) when A is phenyl, X is N, Y is C—OH, and Z¹=Z²=Z³=CH, then at least one of R¹, R², R³, and R⁴ is not —H; and

(v) when A is phenyl, X is CH, Y is CH, Z¹ is CQ³, and Z²=Z³=CH, then either (v-a) Q³ is not unsubstituted or substituted benzyl or (v-b) at least one of R¹, R², R³, and R⁴ is not —H;

or a pharmaceutically acceptable salt thereof.

A third embodiment of the invention is a compound of Formula (I),

wherein

A is

(1) phenyl,

(2) phenyl fused to a carbocycle to form a fused carbocyclic ring system; or

(3) a heterocycle which is:
(i) a 4- to 7-membered saturated or unsaturated monocylic heterocycle which contains from 1 to 4 nitrogen atoms, from zero to 2 heteroatoms selected from oxygen and sulfur, and a balance of carbon atoms, with at least one of the ring atoms being carbon;
(ii) a 7- to 11 -membered fused bicyclic heterocycle either ring of which is saturated or unsaturated, wherein the fused bicyclic heterocycle contains from 1 to 5 nitrogen atoms, from zero to 3 heteroatoms selected from oxygen and sulfur, and a balance of carbon atoms with at least two of the ring atoms being carbon; or
(iii) a 11- to 15-membered fused tricyclic heterocycle any ring of which is saturated or unsaturated, wherein the fused tricyclic heterocycle contains from 1 to 6 nitrogen atoms, from zero to 3 heteroatoms selected from oxygen and sulfur, and a balance of carbon atoms with at least three of the ring atoms being carbon;
   and all other variables are as originally defined above;
   and provided that:

(i) when A is phenyl, X is CH, Y is CH, and Z¹=Z²=Z³=CH, then at least one of R¹, R², R³, and R⁴ is not —H;

(ii) when A is phenyl, X is CH, Y is CQ² wherein Q² is halo or —C₁₋₆ alkyl or phenyl optionally substituted with halo or —C₁₋₆ alkyl or benzyl optionally substituted with halo or —C₁₋₆ alkyl, Z¹=Z²=Z³=CH, and all but one of R¹, R², R³ and R⁴ are independently —H, halo or —C₁₋₆ alkyl, then the other of R¹, R², R³ and R⁴ is not —H, halo or —C₁₋₆ alkyl;

(iii) when A is phenyl, X is CH, Y is CH, Z¹=Z²=Z³=CH, and one of R¹, R², R³, and R⁴ is —CO₂R^{a}, then at least one of the others of R¹, R², R³, and R⁴ is not —H;

(iv) when A is phenyl, X is N, Y is C—OH, and Z¹=Z²=Z³=CH, then at least one of R¹, R², R³, and R⁴ is not —H; and

(v) when A is phenyl, X is CH, Y is CH, Z¹ is CQ³, and Z²=Z³=CH, then either (v-a) Q³ is not unsubstituted or substituted benzyl or (v-b) at least one of R¹, R², R³, and R⁴ is not —H;

or a pharmaceutically acceptable salt thereof.

A fourth embodiment of the present invention is a compound of Formula I, wherein

A is

(1) phenyl,

(2) phenyl fused to a carbocycle to form a fused carbocyclic ring system; or

(3) a heterocycle which is:
(i) a 5- or 6-membered saturated or unsaturated monocylic heterocycle which contains from 1 to 4 nitrogen atoms, from zero to 2 heteroatoms selected from oxygen and sulfur, and a balance of carbon atoms, with at least one of the ring atoms being carbon;
(ii) a 8- to 11-membered fused bicyclic heterocycle either ring of which is saturated or unsaturated, wherein the fused bicyclic heterocycle contains from 1 to 5 nitrogen atoms, from zero to 3 heteroatoms selected from oxygen and sulfur, and a balance of carbon atoms with at least two of the ring atoms being carbon; or
(iii) a 12- to 14-membered fused tricyclic heterocycle any ring of which is saturated or unsaturated, wherein the fused tricyclic heterocycle contains from 1 to 6 nitrogen atoms, from zero to 3 heteroatoms selected from oxygen and sulfur, and a balance of carbon atoms with at least three of the ring atoms being carbon;
   and all other variables are as originally defined above;
   and provided that:

(i) when A is phenyl, X is CH, Y is CH, and Z¹=Z²=Z³=CH, then at least one of R¹, R², R³, and R⁴ is not —H;

(ii) when A is phenyl, X is CH, Y is CQ² wherein Q² is halo or —C₁₋₆ alkyl or phenyl optionally substituted with halo or —C₁₋₆ alkyl or benzyl optionally substituted with halo or —C₁₋₆ alkyl, Z¹=Z²=Z³=CH, and all but one of R¹, R², R³ and R⁴ are independently —H, halo or —C 1-6 alkyl, then the other of R¹, R², R³ and R⁴ is not —H, halo or —C₁₋₆ alkyl;

(iii) when A is phenyl, X is CH, Y is CH, Z¹=Z²=Z³=CH, and one of R¹, R², R³, and R⁴ is —CO₂R^{a}, then at least one of the others of R¹, R², R³, and R⁴ is not —H;

(iv) when A is phenyl, X is N, Y is C—OH, and Z¹=Z²=Z³=CH, then at least one of R¹, R², R³, and R⁴ is not —H; and

(v) when A is phenyl, X is CH, Y is CH, Z¹ is CQ³, and Z²=Z³=CH, then either (v-a) Q³ is not unsubstituted or substituted benzyl or (v-b) at least one of R¹, R², R³, and R⁴ is not —H;

or a pharmaceutically acceptable salt thereof.

A fifth embodiment of the present invention is a compound of Formula I, wherein

A is

(1) phenyl,

(2) a fused carbocyclic ring system selected from the group consisting of

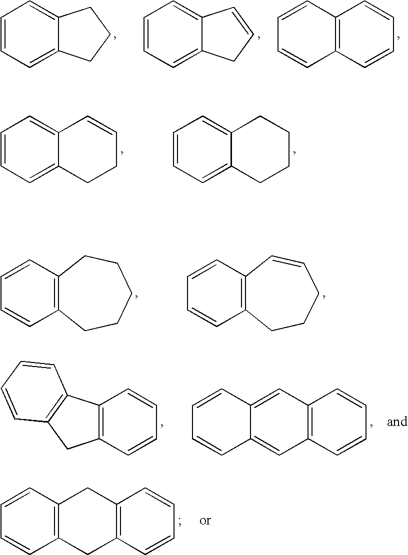

(3) a 5- or 6-membered saturated or unsaturated monocylic heterocycle selected from the group consisting of pyrrolyl, pyrazolyl, imidazolyl, triazolyl, tetrazolyl, pyridyl, pyrazinyl, pyrimidinyl, oxazolyl, thiazolyl, pyrrolidinyl, morpholinyl, piperidinyl, piperazinyl, and thiadiazinanyl;

and all other variables are as originally defined above;

and provided that:

(i) when A is phenyl, X is CH, Y is CH, and Z¹=Z²=Z³=CH, then at least one of R¹, R², R³, and R⁴ is not —H;

(ii) when A is phenyl, X is CH, Y is CQ² wherein Q² is halo or —C₁₋₆ alkyl or phenyl optionally substituted with halo or —C₁₋₆ alkyl or benzyl optionally substituted with halo or —C₁₋₆ alkyl, Z¹=Z²=Z³=CH, and all but one of R, R², R³ and R⁴ are independently —H, halo or —C₁₋₆ alkyl, then the other of R¹, R², R³ and R⁴ is not —H, halo or —C₁₋₆ alkyl;

(iii) when A is phenyl, X is CH, Y is CH, Z¹=Z²=Z³=CH, and one of R¹, R², R³, and R⁴ is —CO₂R^{a}, then at least one of the others of R¹, R², R³, and R⁴ is not —H;

(iv) when A is phenyl, X is N, Y is C—OH, and Z¹=Z²=Z³=CH, then at least one of R¹, R², R³, and R⁴ is not —H; and

(v) when A is phenyl, X is CH, Y is CH, Z¹ is CQ³, and Z²=Z³=CH, then either (v-a) Q³ is not unsubstituted or substituted benzyl or (v-b) at least one of R¹, R², R³, and R⁴ is not —H;

or a pharmaceutically acceptable salt thereof.

A sixth embodiment of the present invention is a compound of Formula I, wherein

X is N;

Y is C-Q²;

Z¹ is C-Q³;

Z² is C-Q⁴;

Z³ is CH;

Q² is

(1) —H,

(2) —C₁₋₆ alkyl,

(3) —C₁₋₆ fluoroalkyl,

(4) —OH,

(5) —O-C₁₋₆ alkyl,

(6) —O—C₁₋₆ fluoroalkyl,

(7) halo,

(8) —CN,

(9) —C₁₋₆ alkyl-OR^{a},

(10) —C₀₋₆ alkyl-C(═O)R^{a},

(11) —C₀₋₆ alkyl-CO₂R^{a},

(12) —C₀₋₆ alkyl-SR^{a},

(13) —N(R^{a})₂,

(14) —C₁₋₆ alkyl —N(R^{a})₂,

(15) —C₀₋₆ alkyl-C(═O)N(R^{a})₂,

(16) —C₁₋₆ alkyl-N(R^{a})—C(R^{a})═O,

(17) —SO₂R^{a},

(18) —N(R^{a})SO₂R^{a},

(19) —C₂₋₅ alkynyl,

(20) —C₂₋₅ alkynyl-CH₂N(R^{a})₂,

(21) —C₂₋₅ alkynyl-CH₂OR^{a},

(22)

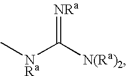

(23) —N(R^{a})—C₁₋₆ alkyl-SR^{a},

(24) —N(R^{a})—C₁₋₆ alkyl-OR^{a},

(25) —N(R^{a})—C₁₋₆ alkyl-N(R^{a})₂,

(26) —N(R^{a})—C₁₋₆ alkyl-N(R^{a})—C(R^{a})═O,

(27) —R^{k},

(28) —C₁₋₆ alkyl substituted with R^{k},

(29) —C₁₋₆ fluoroalkyl substituted with R^{k},

(30) —C₂₋₅ alkenyl-R^{k},

(31) —C₂₋₅ alkynyl-R^{k},

(32) —O—R^{k},

(33) —O—C₁₋₄ alkyl-R^{k},

(34) —S(O)ₙ—R^{k},

(35) —S(O)ₙ—C₁₋₄ alkyl-R^{k},

(36) —O—C₁₋₆ alkyl-OR^{k},

(37) —O—C₁₋₆ alkyl-O—C₁₋₄ alkyl-R^{k},

(38) —O—C₁₋₆ alkyl-SR^{k},

(39) —N(R^{c})—R^{k},

(40) —N(R^{c})—C₁₋₄ alkyl substituted with one or two R^{k} groups,

(41) —N(R^{c})—C₁₋₆ alkyl-OR^{k},

(42) —C(═O)N—C₁₋₆ alkyl-R^{k}, or

(43) —C₂₋₅ alkynyl-CH₂S(O)ₙ—R^{a}; and

each of Q³ and Q⁴:

(1) —H,

(2) —C₁₋₆ alkyl,

(3) —C₁₋₆ fluoroalkyl,

(4) —OH,

(5) —O—C₁₋₆ alkyl,

(6) —O—C₁₋₆ fluoroalkyl,

(7) halo,

(8) —CN,

(9) —C₁₋₆ alkyl-OR^{a},

(10) —C₀₋₆ alkyl-C(═O)R^{a},

(11) —C₁₋₆ alkyl-CO2R^{a},

(12) —SR^{a},

(13) —N(R^{a})₂,

(14) —C₁₋₆ alkyl —N(R^{a})₂,

(15) —C₀₋₆ alkyl-C(═O)N(R^{a})₂,

(16) —SO₂R^{a},

(17) —N(R^{a})SO₂R^{a},

(18) —R^{k}, or

(19) —C₁₋₆ alkyl substituted with R^{k};

and all other variables are as originally defined;

and provided that when A is phenyl, Y is C—OH, and Z¹ are Z² are both CH, then at least one of R¹, R², R³, and R⁴ is not —H;

or a pharmaceutically acceptable salt thereof.

A first class of the present invention is a compound of Formula (I), wherein

Q⁴ is:

(1) —H,

(2) —C₁₋₆ alkyl,

(3) —C₁₋₆ fluoroalkyl,

(4) —O—C₁₋₆ alkyl,

(5) —O—C₁₋₆ fluoroalkyl,

(6) halo,

(7) —CN,

(8) —C₁₋₆ alkyl-OR^{a},

(9) —C₀₋₆ alkyl-C(═O)R^{a},

(10) —C₀₋₆ alkyl-CO₂R^{a},

(11) —SR^{a},

(12) —N(R^{a})₂,

(13) —C₁₋₆ alkyl —N(R^{a})₂,

(14) —C₀₋₆ alkyl-C(═O)N(R^{a})₂,

(15) —SO₂R^{a}, or

(16) —N(R^{a})SO₂R^{a};

and all other variables are as defined in the sixth embodiment;

and provided that when A is phenyl, Y is C—OH, and Z¹ and Z² are both CH, then at least one of R¹, R², R³, and R⁴ is not —H;

or a pharmaceutically acceptable salt thereof.

A second class of the present invention is a compound of Formula (I), wherein Q³ and Q⁴ are both —H;

and all other variables are as defined in the sixth embodiment;

and provided that when A is phenyl, Y is C—OH, then at least one of R¹, R², R³, and R⁴ is not —H;

or a pharmaceutically acceptable salt thereof.

A seventh embodiment of the present invention is a compound of Formula (I), wherein

R¹ is

(1) R^{k},

(2) —C₁₋₆ alkyl substituted with R^{k},

(3) —C₁₋₆ fluoroalkyl substituted with R^{k},

(4) —C₂₋₅ alkenyl-R^{k},

(5) —C₂₋₅ alkynyl-R^{k},

(6) OR^{k},

(7) —O—C₁₋₄ alkyl-R^{k},

(8) —S(O)ₙ—R^{k}, or

(9) —S(O)ₙ—C₁₋₄ alkyl-R^{k}; and

R² is

(1) —H,

(2) —C₁₋₆ alkyl,

(3) —C₁₋₆ fluoroalkyl,

(4) —O—C₁₋₆ alkyl,

(5) —O—C₁₋₆ fluoroalkyl,

(6) —OH,

(7) halo,

(8) —NO₂,

(9) —CN,

(10) —C₁₋₆ alkyl-OR^{a},

(11) —C₀₋₆ alkyl-C(═O)R^{a},

(12) —C₀₋₆ alkyl-CO₂R^{a},

(13) —C₀₋₆ alkyl-SR^{a},

(14) —N(R^{a})₂,

(15) —C₁₋₆ alkyl-N(R^{a})₂,

(16) —C₀₋₆ alkyl-C(═O)N(R^{a})₂,

(17) —C₁₋₆ alkyl-N(R^{a})—C(R^{a})═O,

(18) —SO₂R^{a},

(19) —N(R^{a})SO₂R^{a},

(20) —C₂₋₅ alkenyl,

(21) —O—C₁₋₆ alkyl-OR^{a},

(22) —O—C₁₋₆ alkyl-SR^{a},

(23) —O—C₁₋₆ alkyl-NH—CO₂R^{a},

(24) —O—C₂₋₆ alkyl-N(R^{a})₂,

(25) —N(R^{a})—C₁₋₆ alkyl-SR^{a},

(26) —N(R^{a})—C₁₋₆ alkyl-OR^{a},

(27) —N(R^{a})—C₁₋₆ alkyl-N(R^{a})₂,

(28) —N(R^{a})—C₁₋₆ alkyl-N(R^{a})—C(R^{a})═O,

(29) —R^{k},

(30) —C₁₋₆ alkyl substituted with 1 or 2 R^{k}groups,

(31) —C₁₋₆ fluoroalkyl substituted with 1 or 2 R^{k} groups,

(32) —C₂₋₅ alkenyl-R^{k},

(33) —C₂₋₅ alkynyl-R^{k},

(34) —OR^{k},

(35) —O—C₁₋₄ alkyl-R^{k},

(36) —S(O)ₙ—R^{k},

(37) —S(O)ₙ—C₁₋₄ alkyl-R^{k},

(38) —O—C₁₋₆ alkyl-OR^{k},

(39) —O—C₁₋₆ alkyl-O—C₁₋₄ alkyl-R^{k},

(40) —O—C₁₋₆ alkyl-SR^{k},

(41) —C₁₋₆ alkyl (OR^{b})(R^{k}),

(42) —C₁₋₆ alkyl (OR^{b})(—C₁₋₄ alkyl-R^{k}),

(43) —C₀₋₆ alkyl-N(R^{b})(R^{k}),

(44) —C₀₋₆ alkyl-N(R^{b})(—C₁₋₄ alkyl-R^{k}),

(45) —C₁₋₆ alkyl S(O)ₙ—R^{k},

(46) —C₁₋₆ alkyl S(O)ₙ—C₁₋₄ alkyl-R^{k},

(47) —C₀₋₆ alkyl C(O)—R^{k}, or

(48) —C₀₋₆ alkyl C(O)—C₁4 alkyl-R^{k};

and all other variables are as originally defined;

or a pharmaceutically acceptable salt thereof.

An eighth embodiment of the present invention is a compound of Formula I, wherein

R¹ is

(1) R^{k},

(2) —C₁₋₄ alkyl substituted with R^{k},

(3) —C₁₋₄ fluoroalkyl substituted with R^{k},

(4) —C₂₋₅ alkenyl-R^{k},

(5) —C₂₋₅ alkynyl-R^{k},

(6) —O—R^{k},

(7) —O—C₁₋₄ alkyl-R^{k},

(8) —S(O)ₙ-R^{k}, or

(9) —S(O)ₙ-C₁₋₄ alkyl-R^{k}; and

R² is

(1) —H,

(2) —C₁₋₄ alkyl,

(3) —C₁₋₄ fluoroalkyl,

(4) —O—C₁₋₄ alkyl,

(5) —O—C₁₋₄ fluoroalkyl,

(6) —OH,

(7) halo selected from —F, —Cl and —Br,

(8) —CN,

(9) —C14 alkyl-OR^{a},

(10) —C₀₋₄ alkyl-C(═O)R^{a},

(11) —C₀₋₄ alkyl-C_{O-2}R^{a},

(12) —C₀₋₄ alkyl-SR^{a},

(13) —N(R^{a})₂,

(14) —C₁₋₄ alkyl-N(R^{a})₂,

(15) —C_{O-4} alkyl-C(═O)N(R^{a})²,

(16) —C₁₋₄ alkyl-N(R^{a})—C(R^{a})═O,

(17) —SO₂R^{a},

(18) —N(R^{a})SO₂R^{a},

(19) —O—C₁₋₄ alkyl-OR^{a},

(20) —O —C₁₋₄ alkyl-SR^{a},

(21) —R^{k},

(22) —C₁₋₄ alkyl substituted with R^{k},

(23) —C₁₋₄ fluoroalkyl substituted with R^{k},

(24) —O—R^{k},

(25) —O—C₁₋₄ alkyl-R^{k},

(26) —S(O)ₙ—R^{k},

(27) —S(O)ₙ—C₁₋₄ alkyl-R^{k},

(28) —O—C₁₋₄ alkyl-OR^{k},

(29) —O—C₁₋₄ alkyl-O—C₁₋₄ alkyl-R^{k},

(30) —O—C₁₋₄ alkyl-SR^{k},

(31) —C₁₋₄ alkyl (OR^{b})(R^{k}),

(32) —C₁₋₄ alkyl (OR^{b})(—C₁₋₄ alkyl-R^{k}),

(33) —C₀₋₄ alkyl-N(R^{b})(R^{k}),

(34) —C₀₋₄ alkyl-N(R^{b})(—C₁₋₄ alkyl-R^{k}),

(35) —C₁₋₄ alkyl S(O)ₙ—R^{k},

(36) —C₁₋₄ alkyl S(O)ₙ—C₁₋₄ alkyl-R^{k},

(37) —C₀₋₄ alkyl C(O)—R^{k}, or

(38) —C₀₋₄ alkyl C(O)—C₁₋₄ alkyl-R^{k};

each R^{a} is independently —H or —C₁₋₄ alkyl;

each R^{b} is independently:

(1) —H,

(3) —CF₃,

(4) —R^{k}, or

(5) —(CH₂)₁₋₄-R^{k};

and all other variables are as originally defined;

or a pharmaceutically acceptable salt thereof.

A ninth embodiment of the present invention is a compound of Formula (II): 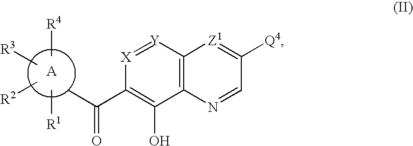
wherein
A is

(1) phenyl,

(2) a fused carbocyclic ring system selected from the group consisting of

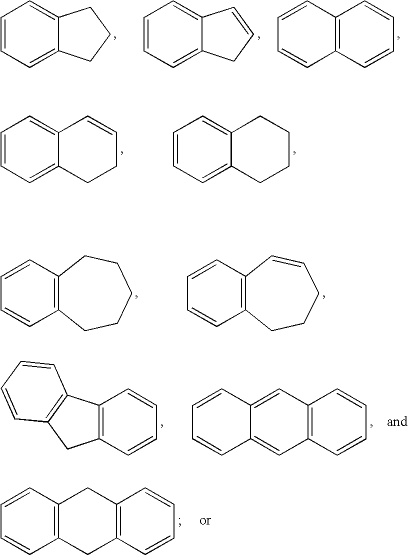

(3) a 5- or 6-membered saturated or unsaturated monocylic heterocycle which contains from 1 to 4 nitrogen atoms, from zero to 2 heteroatoms selected from oxygen and sulfur, and a balance of carbon atoms, with at least one of the ring atoms being carbon;

A is connected by a ring carbon to the exocyclic carbonyl, and is substituted by R¹, R², R³, and R⁴;

X is N or C-Q¹;

Y is N or C-Q², provided that X and Y are not both N;

Z¹ is N or C-Q³;

Q¹ is —H or —C₁₋₄ alkyl;

Q² is

(1) —H,

(2) —C₁₋₄ alkyl,

(3) —C₁₋₄ fluoroalkyl,

(4) —O—C₁₋₄ alkyl,

(5) —O—C₁₋₄ fluoroalkyl,

(6) —OH,

(7) halo,

(8) —CN,

(9) —C₁₋₄ alkyl-OR^{a},

(10) —(CH₂)₀₋₂C(═O)R^{a},

(11) —(CH₂)_{0- 2}CO₂R^{a},

(12) —(CH₂)₀₋₂SR^{a},

(13) —N(R^{a})₂,

(14) —C₁₋₄ alkyl —N(R^{a})₂,

(15) —(CH₂)₀₋₂C(═O)N(R^{a})₂,

(16) —SO₂R^{a},

(17) —N(R^{a})SO₂R^{a},

(18) —C₂₋₃ alkynyl,

(19) —C≡C—CH₂N(R^{a})₂,

(20) —C≡C—CH₂OR^{a}

(21) —N(R^{a})—C₁₋₄ alkyl-SR^{a},

(22) —N(R^{a})—C₁₋₄ alkyl-OR^{a},

(23) —N(R^{a})—C₁₋₄ alkyl-N(R^{a})₂,

(24) —N(R^{a})—C₁₋₄ alkyl-N(R^{a})—C(R^{a})—O,

(25) —R^{k},

(26) —C₁₋₄ alkyl substituted with R^{k},

(27) —C₁₋₄ fluoroalkyl substituted with R^{k},

(28) —C₂₋₅ alkenyl-R^{k},

(29) —C₂₋₅ alkynyl-R^{k},

(30) —O—R^{k},

(31) —O—C₁₋₄ alkyl-R^{k},

(32) —S(O)ₙ—R^{k},

(33) —N(R^{c})—R^{k},

(34) —N(R^{c})—C₁₋₄ alkyl substituted with one or two R^{k}groups,

(35) —N(R^{c})—C₁₋₄ alkyl-OR^{k},

(36) —C(═O)N—C₁₋₄ alkyl-R^{k},

(37) —C═C—CH₂SR^{a}, or

(38) C═C—CH₂SO₂R^{a};

Q³ is

(1) —H,

(2) —C₁₋₄ alkyl,

(3) —C₁₋₄ fluoroalkyl,

(4) —O—C₁₋₄ alkyl,

(5) —O—C₁₋₄ fluoroalkyl,

(6) halo selected from —F, —Cl, and —Br,

(7) —CN,

(8) —C₁₋₄ alkyl-OR^{a}, or

(9) —C₁₋₄ alkyl substituted with R^{k};

Q⁴ is:

(1) —H,

(2) —C₁₋₄ alkyl,

(3) —C₁₋₄ fluoroalkyl,

(4) —O—C₁₋₄ alkyl,

(5) —O—C₁₋₄ fluoroalkyl,

(6) halo selected from —F, —Cl, and —Br,

(7) —CN,

(8) —C16 alkyl-OR^{a},

(9) —N(R^{a})₂, or

(10) —C₁₋₆ alkyl —N(R^{a})₂;

each of R¹ and R² is independently:

(1) —H,

(2) —C₁₋₄ alkyl,

(3) —C₁₋₄ fluoroalkyl,

(4) —O—C₁₋₄ alkyl,

(5) —O—C₁₋₄ fluoroalkyl,

(6) —OH,

(7) halo,

(8) —CN,

(9) —C₁₋₄ alkyl-OR^{a},

(10) —(CH₂)₀₋₂C(═O)R^{a},

(11) —(CH₂)₀₋₂C₀₋₂R^{a},

(12) —(CH₂)₀₋₂SR^{a},

(13) —N(R^{a})₂,

(14) —C₁₋₄ alkyl-N(R^{a})₂,

(15) —(CH₂)₀₋₂C(═O)N(R^{a})₂,

(16) —C₁₋₄ alkyl-N(R^{a})—C(R^{a})═O,

(17) —SO₂R^{a},

(18) —N(R^{a})SO₂R^{a},

(19) —O—C₁₋₄ alkyl-OR^{a},

(20) —O—C₁₋₄ alkyl-SR^{a},

(21) —O—C₁₋₄ alkyl-NH—CO₂R^{a},

(22) —O—C₂₋₄ alkyl-N(R^{a})₂,

(23) —N(R^{a})—C₁₋₄ alkyl-SR^{a},

(24) —N(R^{a})—C₁₋₄ alkyl-OR^{a},

(25) —N(R^{a})—C₁₋₄ alkyl-N(R^{a})₂,

(26) —N(R^{a})—C₁₋₄ alkyl-N(R^{a})—C(R^{a})═O,

(27) R^{k},

(28) —C₁₋₄ alkyl substituted with 1 or 2 R^{k}groups,

(29) —C₁₋₄ fluoroalkyl substituted with 1 or 2 R^{k}groups,

(30) —O—R^{k},

(31) —O—C₁₋₄ alkyl-R^{k},

(32) —S(O)ₙ—R^{k},

(33) —S(O)ₙ—C₁₋₄ alkyl-R^{k},

(34) —O—C₁₋₄ alkyl-OR^{k},

(35) —O—C₁₋₄ alkyl-O—C₁₋₄ alkyl-R^{k},

(36) —O—C₁₋₄ alkyl-SR^{k}, or

(37) —CO₁₋₄ alkyl-N(R^{b})(R^{k});

each of R³ and R⁴ is independently

(1) —H,

(2) halo,

(3) —CN,

(4) —OH,

(5) C₁₋₄ alkyl,

(6) C₁₋₄ fluoroalkyl,

(7) —O—C₁₋₄ alkyl,

(8) —O—C₁₋₄ fluoroalkyl,

(9) —C₁₋₄ alkyl-OR^{a},

(10) —O—C₁₋₄ alkyl-OR^{a},

(11) —O—C₁₋₄ alkyl-SR^{a},

(12) —O—C₁₋₄ alkyl-NH—CO₂R^{a}, or

(13) —O—C₂₋₄ alkyl-N(R^{a})₂;

each R^{a} is independently —H or —C₁₋₄ alkyl;

each R^{b} is independently:

(1) —H,

(2) —C₁₋₄ alkyl,

(3) —C₁₋₄ fluoroalkyl,

(4) R^{k},

(5) —C₁₋₄ alkyl-R^{k},

(6) —S(O)ₙ-R^{k}, or

(7) —C(═O)-R^{k};

each R^{c} is independently

(1) —H,

(2) —C₁₋₄ alkyl,

(3) —C₁₋₄ alkyl substituted with —N(R^{a})₂, or

(4) —C₁₋₄ alkyl-phenyl, wherein the phenyl is optionally substituted with 1 to 3 substituents independently selected from halogen, C₁₋₄ alkyl, C₁₋₄ fluoroalkyl, —O—C₁₋₄ alkyl, —O—C₁₋₄ fluoroalkyl, —S—C₁₋₄ alkyl, —CN, and —OH;

each R^{k} is independently:

(1) aryl selected from phenyl and naphthyl, wherein aryl is unsubstituted or substituted with from I to 5 substituents independently selected from:
(a) halogen,
(b) C₁₋₆ alkyl,
(c) C₁₋₆ fluoroalkyl,
(d) —O—C₁₋₆ alkyl,
(e) —O—C₁₋₆ fluoroalkyl,
(f) phenyl,
(g) —S—C₁₋₆ alkyl,
(h) —CN,
(i) —OH,
(j) phenyloxy, unsubstituted or substituted with from 1 to 3 substituents independently selected from:
   (i) halogen,
   (ii) C₁₋₆ alkyl,
   (iii) C₁₋₆ fluoroalkyl, and
   (iv) —OH,
(k) —N(R^{a})₂,
(l) —C₁₋₆ alkyl-N(R^{a})₂,
(m) R^{t},
(p) —(CH₂)₀₋₃C(═O)N(R^{a})₂, and
(q) —(CH₂)₀₋₃C(═O)R^{a};

(2) —C₃₋₇ cycloalkyl, unsubstituted or substituted with from 1 to 3 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,,
(f) —CN,
(h) phenyl, and
(j) —OH;

(3) —C₃₋₇ cycloalkyl fused with a phenyl ring, unsubstituted or substituted with from 1 to 5 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,
(f) —CN, and
(g) —OH;

(4) a 5- or 6- membered heteroaromatic ring containing from 1 to 4 heteroatoms independently selected from oxygen, nitrogen and sulfur, wherein the heteroaromatic ring is unsubstituted or substituted on nitrogen or carbon with from 1 to 5 substituents independently selected from:
(a) halogen,
(b) C₁₋₆ alkyl,
(c) C₁₋₆ fluoroalkyl,
(d) —O—C₁₋₆ alkyl,
(e) —O—C₁₋₆ fluoroalkyl,
(f) phenyl,
(g) —S—C₁₋₆ alkyl,
(h) —CN,
(i) —OH,
(j) phenyloxy, unsubstituted or substituted with from 1 to 3 substituents independently selected from:
   (i) halogen,
   (ii) C₁₋₆ alkyl,
   (iii) C₁₋₆ fluoroalkyl, and
   (iv) —OH,
(k) —N(R^{a})₂,
(l) —C₁₋₆ alkyl-N(R^{a})₂,
(m) R^{t},
(n) oxo,
(o) —(CH₂)₀₋₃C(═O)N(R^{a})₂, and
(p) —(CH2)₀₋₃C(═O)R^{a};

(5) a 5- or 6- membered saturated heterocyclic ring containing 1 or 2 heteroatoms independently selected from oxygen, nitrogen and sulfur, wherein the heterocyclic ring is unsubstituted or substituted with from 1 to 4 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,
(f) —CN,
(g) oxo,
(h) phenyl,
(i) benzyl,
(j) phenylethyl,
(k) —OH,
(l) —(CH₂)₀₋₃C(═O)N(R^{a})₂,
(m) —(CH₂)₀₋₃C(═O)R^{a},
(n) —N(R^{a})—C(═O)R^{a},
(o) —N(R^{a})-C(=O)OR^{a},
(p) —(CH₂)₁₋₃N(R^{a})—C(═O)R^{a},
(q) —N(R^{a})₂,
(r) —(CH₂)₁₋₃N(R^{a})₂,
(s) —(CH₂)₀₋₃C(═O)R^{t},
(t) —R^{t},
(u) —N(R^{a})R^{t}, and
(v) —(CH₂)₁₋₃R^{t}; or

(6) an 8- to 10-membered heterobicyclic ring containing from 1 to 4 heteroatoms independently selected from oxygen, nitrogen and sulfur, wherein the heterobicyclic ring is saturated or unsaturated, and is unsubstituted or substituted with from 1 to 5 substituents independently selected from:
(a) halogen,
(b) C₁₋₆ alkyl,
(c) —O—C16 alkyl,
(d) C₁₋₆ fluoroalkyl,
(e) —O—C₁₋₆ fluoroalkyl,
(f) —CN,
(g) ═O, and
(h) —OH;
   R^{t} is naphthyl or a 5- or 6-membered heteromonocylic ring containing from 1 to 4 nitrogen atoms, wherein the heteromonocyclic ring is saturated or unsaturated, and wherein the naphthyl or the heteromonocyclic ring is unsubstituted or substituted with 1 or 2 substituents independently selected from halogen, oxo, C₁₋₄ alkyl, and —O—C₁₋₄ alkyl; and
   n is an integer equal to 0, 1 or 2;
   and provided that:

(i) when A is phenyl, X is CH, Y is CH, Z¹ is CH, and Q⁴ is —H, then at least one of R¹, R², R³, and R⁴ is not —H;

(ii) when A is phenyl, X is CH, Y is CQ² wherein Q² is halo or —C₁₋₆ alkyl or phenyl optionally substituted with halo or —C₁₋₆ alkyl or benzyl optionally substituted with halo or —C₁₋₆ alkyl, Z¹ is CH, Q⁴ is —H, and all but one of R¹, R², R³ and R⁴ are independently —H, halo or —C₁₋₆ alkyl, then the other of R¹, R², R³ and R⁴ is not —H, halo or —C₁₋₆ alkyl;

(iii) when A is phenyl, X is CH, Y is CH, Z¹ is CH, Q⁴ is —H, and one of R¹, R², R³, and R⁴ is —CO₂R^{a}, then at least one of the others of R¹, R², R³, and R⁴ is not —H;

(iv) when A is phenyl, X is N, Y is C—OH, Z¹ is CH, and Q⁴ is —H, then at least one of R¹, R², R³, and R⁴ is not —H; and

(v) when A is phenyl, X is CH, Y is CH, Z¹ is CQ³, and Q⁴ is —H, then either (v-a) Q³ is not unsubstituted or substituted benzyl or (v-b) at least one of R¹, R², R³, and R⁴ is not —H;

or a pharmaceutically acceptable salt thereof.

A tenth embodiment of the present invention is a compound of Formula II, wherein

X is N;

Y is C-Q²;

Z¹ is C-Q³;

Q² is

(1) —H,

(2) —C₁₋₄ alkyl,

(3) —(CH₂)₀₋₂CF₃,

(4) —OH,

(5) —O—C₁₋₄ alkyl,

(6) —O—(CH₂)₀₋₂CF₃,

(7) halo selected from —F, —Cl and —Br,

(8) —CN,

(9) —(CH₂)₁₋₃OR^{a},

(10) —(CH₂)₀₋₂C(═O)R^{a},

(11) —(CH₂)₀₋₂CO₂R^{a},

(12) —(CH₂)₀₋₂SR^{a},

(13) —N(R^{a})₂,

(14) —(CH₂)₁₋₃N(R^{a})₂,

(15) —(CH₂)₀₋₂C(═O)N(R^{a})₂,

(16) —SO₂R^{a},

(17) —N(R^{a})SO₂R^{a},

(18) —C≡C—CH₂OR^{a},

(19) —N(R^{a})—(CH₂)₁₋₄SR^{a},

(20) —N(R^{a})—(CH₂)₁₋₄OR^{a},

(21) —N(R^{a})—(CH₂)₁₋₄—N(R^{a})₂,

(22) —N(R^{a})—(CH₂)₁₋₄N(R^{a})—C(R^{a})═O,

(23) —R^{k},

(24) —(CH₂)₁₄R^{k},

(25) —C═C—CH₂R^{k}

(26) —O—R^{k},

(27) —S(O)ₙ—R^{k},

(28) —N(R_{c})—R^{k},

(29) —N(R_{c})—(CH₂)₁₄H substituted with one or two R^{k}groups,

(29) —N(R_{c})—(CH₂)₁₋₄OR^{k},

(30) —C(═O)N—(CH₂)₁₋₄R^{k},

(31) —C≡C—CH₂SR^{a}, or

(32) —C≡C—CH₂SO₂R^{a};

Q³ is —H or —C₁₋₄ alkyl;

Q⁴ is —H;

R¹ is

(1) —R^{k},

(2) —(CH₂)₁₋₄H substituted with R^{k},

(3) —O—R^{k},

(4) —O—(CH₂)₁₋₄—C₁₋₄ alkyl-R^{k},

(5) —S(O)ₙ—R^{k},

(6) —S(O)ₙ—(CH₂)₁₋₄—R^{k},

(7) —O—(CH₂)₁₋₄—OR^{k},

(8) —O—(CH₂)₁₋₄—O—(CH₂)₁₋₄—R^{k},

(9) —O—(CH₂)₁₋₄—SR^{k}, or

(10) —(CH₂)₀₋₄—N(R^{b})(R^{k});

R² is

(1) —H,

(2) —C₁₋₄ alkyl,

(3) —(CH₂)₀₋₂CF₃,

(4) —O—C₁₋₄ alkyl,

(5) —O—(CH₂)₀₋₂CF₃,

(6) —OH,

(7) halo selected from —F, —Cl and —Br,

(8) —CN,

(9) —(CH₂)₁₋₃OR^{a},

(10) —(CH₂)₀₋₂C(═O)R^{a},

(11) —(CH₂)₀₋₂CO₂R^{a},

(12) —(CH₂)₀₋₂SR^{a},

(13) —N(R^{a})₂,

(14) —(CH₂)₁₋₃N(R^{a})₂,

(15) —(CH₂)₀₋₂C(═O)N(R^{a})₂,

(16) —C₁₋₄ alkyl-N(R^{a})—C(R^{a})═O,

(17) —SO₂R^{a},

(18) —N(R^{a})SO₂R^{a},

(19) —O—(CH₂)₁₋₄OR^{a},

(20) —O—(CH₂)₁₋₄SR^{a},

(21) —O—(CH₂)₁₋₄NH—CO₂R^{a},

(22) —O—(CH₂)₂₋₄N(R^{a})₂,

(23) —N(R^{a})—(CH₂)₁₋₄SR^{a},

(24) —N(R^{a})—(CH₂)₁₋₄OR^{a},

(25) —N(R^{a})—(CH₂)₁₋₄N(R^{a})₂,

(26) —N(R^{a})—(CH₂)₁₋₄N(R^{a})—C(R^{a})═O,

(27) —R^{k},

(28) —(CH₂)₁₋₄H substituted with R^{k},

(29) —O—R^{k},

(30) —O—(CH₂)₁₋₄R^{k},

(31) —S(O)ₙ—R^{k},

(32) —S(O)ₙ—(CH₂)₁₋₄R^{k},

(33) —O—(CH₂)₁₋₄OR^{k},

(34) —O—(CH₂)₁₋₄—O—(CH₂)₁₋₄R^{k},

(35) —O—(CH₂)₁₋₄SR^{k}, or

(36) —(CH₂)₀₋₄N(R^{b})(R^{k});

each of R³ and R⁴ is independently

(1) —H,

(2) halo selected from —F, —Cl and —Br,

(3) —CN,

(4) —OH,

(5) C₁₋₄ alkyl,

(6) —(CH₂)₀₋₂CF₃,

(7) —O—C₁₋₄ alkyl, or

(8) —O(CH₂)₀₋₂CF₃,

each R^{a} is independently —H or —C₁₋₄ alkyl;

each R^{b} is independently:

(1) —H,

(2) —C₁₋₄ alkyl,

(3) —CF₃,

(4) —R^{k}, or

(5) —(CH₂)₁₋₄—R^{k};

each R^{c} is independently

(1) —H,

(2) —C₁₋₄ alkyl,

(3) —(CH₂)₁₋₄N(R^{a})₂, or

(4) —(CH₂)₁₋₄-phenyl, wherein the phenyl is optionally substituted with 1 to 3 substituents independently selected from halogen, C₁₋₄ alkyl, C₁₋₄ fluoroalkyl, —O—C₁₋₄ alkyl, —O—C₁₋₄ fluoroalkyl, —S—C₁₋₄ alkyl, —CN, and —OH; and

each R^{k} is independently:

(1) aryl selected from phenyl and naphthyl, wherein aryl is unsubstituted or substituted with from I to 4 substituents independently selected from:
(a) halogen,
(b) C₁₋₄ alkyl,
(c) C₁₋₄ fluoroalkyl,
(d) —O—C₁₋₄ alkyl,
(e) —O—C₁₋₄ fluoroalkyl,
(f) phenyl,
(g) —S—C₁₋₄ alkyl,
(h) —CN,
(i) —OH,
(j) phenyloxy, unsubstituted or substituted with from 1 to 3 substituents independently selected from:
   (i) halogen,
   (ii) C₁₋₄ alkyl,
   (iii) C₁₋₄ fluoroalkyl, and
   (iv) —OH,
(k) —N(R^{a})₂,
(l) —C₁₋₄ alkyl-N(R^{a})₂,
(m) R^{t},
(p) —(CH₂)₀₋₃C(═O)N(R^{a})₂, and
(q) —(CH₂)₀₋₃C(═O)R^{a};

(2) —C₃₋₆ cycloalkyl, unsubstituted or substituted with from 1 to 3 substituents independently selected from:
(a) halogen,
(b) C₁₋₄ alkyl,
(c) —O—C₁₋₄ alkyl,
(d) C₁₋₄ fluoroalkyl,
(e) —O—C₁₋₄ fluoroalkyl,
(f) —CN,
(h) phenyl, and
(j) —OH;

(3) —C₃₋₆ cycloalkyl fused with a phenyl ring, unsubstituted or substituted with from 1 to 4 substituents independently selected from:
(a) halogen,
(b) C₁₋₄ alkyl,
(c) —O—C₁₋₄ alkyl,
(d) C₁₋₄ fluoroalkyl,
(e) —O—C₁₋₄ fluoroalkyl,
(f) —CN, and
(g) —OH;

(4) a 5- or 6-membered heteroaromatic ring selected from thienyl, pyridyl, imidazolyl, pyrrolyl, pyrazolyl, thiazolyl, isothiazolyl, oxazolyl, isooxazolyl, pyrazinyl, pyirimidinyl, triazolyl, tetrazolyl, furanyl, and pyridazinyl, wherein the heteroaromatic ring is unsubstituted or substituted on nitrogen or carbon with from 1 to 4 substituents independently selected from:
(a) halogen,
(b) C₁₋₄ alkyl,
(c) C₁₋₄ fluoroalkyl,
(d) —O—C₁₋₄ alkyl,
(e) —O—C₁₋₄ fluoroalkyl,
(f) phenyl,
(g) —S—C₁₋₄ alkyl,
(h) —CN,
(i) —OH,
(j) phenyloxy, unsubstituted or substituted with from 1 to 3 substituents independently selected from:
   (i) halogen,
   (ii) C₁₋₄ alkyl,
   (iii) C₁₋₄ fluoroalkyl, and
   (iv) —OH,
(k) —N(R^{a})₂,
(l) —C₁₋₄ alkyl-N(R^{a})₂,
(m) R^{t},
(n) oxo,
(o) —(CH₂)₀₋₃C(═O)N(R^{a})₂, and
(p) —(CH₂)₀₋₃C(═O)R^{a};

(5) a 5- or 6-membered saturated heterocyclic ring selected from piperidinyl, morpholinyl, thiomorpholinyl, thiazolidinyl, isothiazolidinyl, oxazolidinyl, isooxazolidinyl, pyrrolidinyl, imidazolidinyl, piperazinyl, tetrahydrofuranyl, and pyrazolidinyl, wherein the heterocyclic ring is unsubstituted or substituted with from 1 to 3 substituents independently selected from:
(a) halogen,
(b) C₁₋₄ alkyl,
(c) —O—C₁₋₄ alkyl,
(d) C₁₋₄ fluoroalkyl,
(e) —O—C₁₋₄ fluoroalkyl,
(f) —CN,
(g) ═O,
(h) phenyl,
(i) benzyl,
(j) phenylethyl,
(k) —OH,
(l) —(CH₂)₀₋₃C(═O)N(R^{a})₂,
(m) —(CH₂)₀₋₃C(═O)R^{a},
(n) N(R^{a})—C(═O)R^{a},
(o) N(R^{a})—C(═O)OR^{a},
(p) (CH₂)₁₋₃N(R^{a})—C(═O)R^{a},
(q) N(R^{a})₂,
(r) (CH₂)₁₋₃N(R^{a})₂,
(s) —(CH₂)₀₋₃C(═O)R^{t},
(t) —R^{t},
(u) —N(R^{a})R^{t}, and
(v) —(CH₂)₁₋₃R^{t}; or

(6) an 8- to 10- membered heterobicyclic ring selected from indolyl, benzotriazolyl, benzoimidazolyl, imidazo[4,5-b]pyridinyl, dihydroimidazo[4,5-b]pyridinyl, pyrazolo[4,3-c]pyridinyl, dihydropyrazolo[4,3-c]pyridinyl, tetrahydropyrazolo[4,3-c]pyridinyl, pyrrolo[1,2-a]pyrazinyl, dihydropyrrolo[1 ,2-a]pyrazinyl, tetrahydropyrrolo[1,2-a]pyrazinyl, octahydropyrrolo[1,2-a]pyrazinyl, isoindolyl, indazolyl, indolinyl, isoindolinyl, quinolinyl, isoquinolinyl, quinoxalinyl, quinazolinyl, cinnolinyl, chromanyl, and isochromanyl, wherein the bicyclic ring is unsubstituted or substituted with 1 or 2 substituents independently selected from:
(a) halogen,
(b) C₁₋₄ alkyl,
(c) —O—C₁₋₄ alkyl,
(d) C₁₋₄ fluoroalkyl,
(e) —O—C₁₋₄ fluoroalkyl,
(f) —CN,
(g) ═O, and
(h) —OH; and
   R^{t} is naphthyl or a 5- or 6-membered heteromonocylic ring selected from pyrrolidinyl, pyrazolidinyl, imidazolinyl, piperidinyl, piperazinyl, pyrrolyl, pyridyl, imidazolyl, pyrazolyl, triazolyl, tetrazolyl, pyrazinyl, pyrimidinyl, and pyradizinyl; and wherein the naphthyl or the heteromonocyclic ring is unsubstituted or substituted with 1 or 2 substituents independently selected from halogen, oxo, C₁₋₄ alkyl, and —O—C₁₋₄ alkyl;
   or a pharmaceutically acceptable salt thereof.

An eleventh embodiment of the present invention is a compound of Formula III: 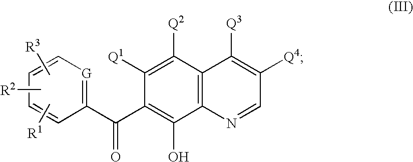
wherein G is N or is CH optionally substituted with one of R¹, R², and R³;
and all other variables are as originally defined;
and provided that:

(i) when G is not N and Q¹=Q²=Q³=Q⁴=H, then at least one of R¹, R² and R³ is not —H;

(ii) when G is not N, Q¹ is H, Q² is halo or —C₁₋₆ alkyl or phenyl optionally substituted with halo or —C₁₋₆ alkyl or benzyl optionally substituted with halo or —C₁₆ alkyl, Q³=Q⁴=H, and all but one of R¹, R²,and R³ are independently —H, halo or —C₁₋₆ alkyl, then the other of R¹, R², and R³ is not —H, halo or —C₁₋₆ alkyl;

(iii) when G is not N, Q¹=Q²=Q³=Q⁴=H, and one of R¹, R², and R³ is —CO₂R^{a}, then at least one of the others of R¹, R² and R³ is not —H; and

(iv) when G is not N and Q¹=Q²=Q⁴=H, then either (v-a) Q³ is not unsubstituted or substituted benzyl or (v-b) at least one of R¹, R² and R³ is not —H;

or a pharmaceutically acceptable salt thereof.

A twelfth embodiment of the present invention is a compound of Formula (III), wherein

each of Q¹ and Q⁴ is —H;

Q² is

(1) —H,

(2) methyl,

(3) ethyl,

(4) CF₃,

(5) —OH,

(6) methoxy,

(7) ethoxy

(8) —OCF₃

(9) halo selected from —F, —Cl and —Br,

(10) —CN,

(11) —CH₂OH,

(12) —CH₂OCH₃

(13) —SR^{a},

(14) —N(R^{a})₂,

(15) —SO₂R^{a},

(16) —C≡C—CH₂OR^{a}

(17) —N(R^{a})—(CH₂)₁₋₃SR^{a},

(18) —N(R^{a})—(CH₂)₁₋₃OR^{a},

(19) —N(R^{a})—(CH₂)₁₋₃N(R^{a})₂,

(20) —N(R^{a})—(CH2)₁₋N(R^{a})—C(R^{a})═O,

(21) —R^{k},

(22) —(CH₂)₁₋₄R^{k},

(23) —C≡C—CH₂R^{k},

(24) —O—R^{k},

(25) —S—R^{k},

(26) —SO₂—R^{k},

(27) —N(R^{c})—R^{k},

(28) —N(R^{c})—(CH₂)₁₋₄R^{k},

(29) —N(R^{c})—(CH₂)₁₋₄OR^{k},

(30) —C(═O)N—(CH₂)₁₋₄R^{k},

(31) —C≡C—CH₂SR^{a},or

(32) —C≡C—CH₂SO₂R^{a};

Q³ is —H or —C₁₋₄ alkyl;

each of R¹ and R² is independently:

(1) —H,

(2) methyl,

(3) ethyl,

(4) CF₃,

(5) methoxy,

(6) ethoxy

(7) —OCF3

(8) halo selected from —F, —Cl and —Br,

(9) —CN,

(10) —CH₂OR^{a},

(11) —CO₂R^{a},

(12) —SR^{a},

(13) —N(R^{a})₂,

(14) —(CH₂)₁₋₃N(R^{a})₂,

(15) —SO₂R^{a},

(16) —(CH₂)₁₋₂N(R^{a})—C(R^{a})═O,

(17) —R^{k},

(18) —(CH₂)₁₋₄R^{k},

(19) —O—R^{k}, or

(20) —O—(CH₂)₁₋₄R^{k},

R³ is —H;

each R^{a} is independently —H or —C₁₋₄ alkyl;

each R^{c} is independently

(1) —H,

(2) —C₁₋₄ alkyl,

(3) —(CH₂)₁₋₄N(R^{a})₂, or

(4) —(CH₂)₁₋₄-phenyl, wherein the phenyl is optionally substituted with 1 to 3 substituents independently selected from halogen, C₁₋₄ alkyl, C₁₋₄ fluoroalkyl, —O—C₁₋₄ alkyl, —O—C₁₋₄ fluoroalkyl, —S—C₁₋₄ alkyl, —CN, and —OH; and

each R^{k} is independently:

(1) aryl selected from phenyl and naphthyl, wherein aryl is unsubstituted or substituted with from 1 to 4 substituents independently selected from:
(a) halogen,
(b) C₁₋₄ alkyl,
(c) C₁₋₄ fluoroalkyl,
(d) —O—C₁₋₄ alkyl,
(e) —O—C₁₋₄ fluoroalkyl,
(f) phenyl,
(g) —S—C₁₋₄ alkyl,
(h) —CN,
(i) —OH,
(j) phenyloxy, unsubstituted or substituted with from 1 to 3 substituents independently selected from:
   (i) halogen,
   (ii) C₁₋₄ alkyl,
   (iii) C₁₋₄ fluoroalkyl, and
   (iv) —OH,
(k) —N(R^{a})₂,
(l) —C₁₋₄ alkyl-N(R^{a})₂,
(m) —R^{t},
(p) —(CH₂)₀₋₃C(═O)N(R^{a})₂, and
(q) —(CH₂)₀₋₃C(═O)R^{a};

(2) —C₃₋₆ cycloalkyl, unsubstituted or substituted with from 1 to 3 substituents independently selected from:
(a) halogen,
(b) C₁₋₄ alkyl,
(c) —O—C₁₋₄ alkyl,
(d) C₁₋₄ fluoroalkyl,
(e) —O—C₁₋₄ fluoroalkyl,
(f) —CN,
(h) phenyl, and
(j) —OH;

(3) —C₃₋₆ cycloalkyl fused with a phenyl ring, unsubstituted or substituted with from 1 to 4 substituents independently selected from:
(a) halogen,
(b) C₁₋₄ alkyl,
(c) —O—C₁₋₄ alkyl,
(d) C₁₋₄ fluoroalkyl,
(e) —O—C₁₋₄ fluoroalkyl,
(f) —CN, and
(g) —OH;

(4) a 5- or 6-membered heteroaromatic ring selected from thienyl, pyridyl, imidazolyl, pyrrolyl, pyrazolyl, thiazolyl, isothiazolyl, oxazolyl, isooxazolyl, pyrazinyl, pyirimidinyl, triazolyl, tetrazolyl, furanyl, and pyridazinyl, wherein the heteroaromatic ring is unsubstituted or substituted on nitrogen or carbon with from 1 to 4 substituents independently selected from:
(a) halogen,
(b) C₁₋₄ alkyl,
(c) C₁₋₄ fluoroalkyl,
(d) —O—C₁₋₄ alkyl,
(e) —O—C₁₋₄ fluoroalkyl,
(f) phenyl,
(g) —S—C₁₋₄ alkyl,
(h) —CN,
(i) —OH,
(j) phenyloxy, unsubstituted or substituted with from 1 to 3 substituents independently selected from:
   (i) halogen,
   (ii) C₁₋₄ alkyl,
   (iii) C₁₋₄ fluoroalkyl, and
   (iv) —OH,
(k) —N(R^{a})₂,
(l) —C₁₋₄ alkyl-N(R^{a})₂,
(m) —R^{t},
(n) oxo,
(o) —(CH₂)₀₋₃C(═O)N(R^{a})₂, and
(p) —(CH₂)₀₋₃C(═O)R^{a};

(5) a 5- or 6-membered saturated heterocyclic ring selected from piperidinyl, morpholinyl, thiomorpholinyl, thiazolidinyl, isothiazolidinyl, oxazolidinyl, isooxazolidinyl, pyrrolidinyl, imidazolidinyl, piperazinyl, tetrahydrofuranyl, and pyrazolidinyl, wherein the heterocyclic ring is unsubstituted or substituted with from 1 to 3 substituents independently selected from:
(a) halogen,
(b) C₁₋₄ alkyl,
(c) —O—C₁₋₄ alkyl,
(d) C₁₋₄ fluoroalkyl,
(e) —O—C₁₋₄ fluoroalkyl,
(f) —CN,
(g) ═O,
((h) phenyl,
(i) benzyl,
(j) phenylethyl,
(k) —OH,
(l) —(CH₂)₀₋₃C(═O)N(R^{a})₂,
(m) —(CH₂)₀₋₃C(═O)R^{a},
(n) N(R^{a})—C(═O)R^{a},
(o) N(R^{a})—C(═O)OR^{a},
(p) (CH₂)₁₋₃N(R^{a})—C(═O)R^{a},
(q) N(R^{a})₂,
(r) (CH₂)₁₋₃N(R^{a})₂,
(s) —(CH₂)₀₋₃C(═O)R^{t},
(t) —R^{t},
(u) —N(R^{a})R^{t}, and
(v) —(CH₂)₁₋₃R^{t}; or

(6) an 8- to 10-membered heterobicyclic ring selected from indolyl, benzotriazolyl, benzoimidazolyl, imidazo[4,5-b]pyridinyl, dihydroimidazo[4,5-b]pyridinyl, pyrazolo[4,3-c]pyridinyl, dihydropyrazolo[4,3-c]pyridinyl, tetrahydropyrazolo[4,3-c]pyridinyl, pyrrolo[1,2-a]pyrazinyl, dihydropyrrolo[1,2-a]pyrazinyl, tetrahydropyrrolo[1,2-a]pyrazinyl, octahydropyrrolo[1,2-a]pyrazinyl, isoindolyl, indazolyl, indolinyl, isoindolinyl, quinolinyl, isoquinolinyl, quinoxalinyl, quinazolinyl, cinnolinyl, chromanyl, and isochromanyl, wherein the bicyclic ring is unsubstituted or substituted with 1 or 2 substituents independently selected from:
(a) halogen,
(b) C₁₋₄ alkyl,
(c) —O—C₁₋₄ alkyl,
(d) C₁₋₄ fluoroalkyl,
(e) —O—C₁₋₄ fluoroalkyl,
(f) —CN,
(g) ═O, and
(h) —OH;
   R^{t} is naphthyl or a 5- or 6-membered heteromonocylic ring selected from pyrrolidinyl, pyrazolidinyl, imidazolinyl, piperidinyl, piperazinyl, pyrrolyl, pyridyl, imidazolyl, pyrazolyl, triazolyl, tetrazolyl, pyrazinyl, pyrimidinyl, and pyradizinyl; and wherein the naphthyl or the heteromonocyclic ring is unsubstituted or substituted with 1 or 2 substituents independently selected from halogen, oxo, C₁4 alkyl, and —O—C₁₄ alkyl;
   and provided that:

(i) when G is not N, Q² is H, and Q³ is H, then at least one of R¹ and R² is not —H;

(ii) when G is not N, Q² is halo or methyl or ethyl or phenyl optionally substituted with halo or —C₁₋₄ alkyl or benzyl optionally substituted with halo or —C₁₋₄ alkyl, and Q³ is H, then at least one of R¹ and R² is not —H, halo, methyl or ethyl; and

(iii) when G is not N, Q² is H, and Q³ is H, and one of R¹ and R² is —CO₂R^{a}, then the other of R¹ and R² is not —H;

or a pharmaceutically acceptable salt thereof.

A third class of the present invention is a compound of Formula III, wherein

R¹ is:

(1) —R^{k},

(2) —(CH₂)₁₋₄R^{k},

(3) —O—R^{k}, or

(4) —O—(CH₂)₁₋₄R^{k};

R² is:

(1) —H,

(2) methyl,

(3) ethyl,

(4) CF₃,

(5) methoxy,

(6) ethoxy

(7) —OCF₃

(8) halo selected from —F, —Cl and —Br,

(9) —CN,

(10) —CH₂OR^{a},

(11) —CO₂R^{a},

(12) —SR^{a},

(13) —N(R^{a})₂,

(14) —(CH₂)₁₋₃N(R^{a})₂,

(15) —SO₂R^{a},

(16) —(CH₂)₁₋₂N(R^{a})—C(R^{a})═O,

(17) —R^{k},

(18) —(CH₂)₁₄R^{k},

(19) —O—R^{k}, or

(20) —O—(CH₂)₁₄R^{k},

each R^{c} is independently —H or —C₁₋₄ alkyl;

each R^{k} is independently:

(1) phenyl which is unsubstituted or substituted with from 1 to 4 substituents independently selected from:
(a) halogen selected from -F, —Cl, and -Br,
(b) methyl,
(c) —CF₃,
(d) methoxy,
(e) —OCF₃,
(f) phenyl,
(g) —S—CH₃,
(h) —CN,
(i) —OH,
(j) phenyloxy, unsubstituted or substituted with from 1 to 3 substituents independently selected from:
   (i) halogen selected from —F, —Cl, and —Br,
   (ii) methyl,
   (iii) —CF₃, and
   (iv) —OH,
(k) —N(R^{a})₂,
(l) —(CH₂)₁₋₃N(R^{a})₂,
(m) —R^{t},
(p) —(CH₂)₀₋₃C(═O)N(R^{a})₂, and
(q) —(CH₂)₀₋₃C(═O)R^{a};

(2) —C₃₋₆ cycloalkyl, unsubstituted or substituted with from 1 to 3 substituents independently selected from:
(a) halogen selected from —F, —Cl, and —Br,
(b) methyl,
(c) —CF₃,
(d) methoxy,
(e) —OCF₃,
(f) —CN,
(h) phenyl, and
(j) —OH;

(3) a 5- or 6-membered heteroaromatic ring selected from thienyl, pyridyl, imidazolyl, pyrrolyl, pyrazolyl, thiazolyl, isothiazolyl, oxazolyl, isooxazolyl, pyrazinyl, pyirimidinyl, triazolyl, tetrazolyl, furanyl, and pyridazinyl, wherein the heteroaromatic ring is unsubstituted or substituted on nitrogen or carbon with 1 or 2 substituents independently selected from:
(a) halogen selected from —F, —Cl, and —Br,
(b) methyl,
(c) —CF₃,
(d) methoxy,
(e) —OCF₃,
(f) phenyl,
(g) —S—C₁₋₆ alkyl,
(h) —CN,
(i) —OH,
(j) phenyloxy, unsubstituted or substituted with from 1 to 3 substituents independently selected from:
   (i) halogen selected from —F, —Cl, and —Br,
   (ii) methyl,
   (iii) —CF₃, and
   (iv) —OH,
(k) —N(R^{a})₂,
(l) —C₁₋₆ alkyl-N(R^{a})₂,
(m) —R^{t},
(n) oxo,
(o) —(CH₂)₀₋₃C(═O)N(R^{a})₂, and
(p) —(CH₂)₀₋₃C(═O)R^{a}; and

(4) a 5- or 6-membered saturated heterocyclic ring selected from piperidinyl, morpholinyl, thiomorpholinyl, thiazolidinyl, isothiazolidinyl, oxazolidinyl, isooxazolidinyl, pyrrolidinyl, imidazolidinyl, piperazinyl, tetrahydrofuranyl, and pyrazolidinyl, wherein the heterocyclic ring is unsubstituted or substituted with 1 or 2 substituents independently selected from:
(a) halogen selected from —F, —Cl, and —Br,
(b) methyl,
(c) —CF₃,
(d) methoxy,
(e) —OCF₃,
(f) —CN,
(g) ═O,
(h) phenyl,
(i) benzyl,
(j) phenylethyl,
(k) —OH,
(l) —(CH₂)₀₋₃C(═O)N(R^{a})₂,
(m) —(CH₂)₀₋₃C(═O)R^{a},
(n) N(R^{a})—C(═O)R^{a},
(o) N(R^{a})—C(═O)OR^{a},
(p) N(R^{a})—C(═O)OC(CH₃)₃,
(q) (CH₂)₁₋₃N(R^{a})—C(═O)R^{a},
(r) N(R^{a})₂,
(s) (CH₂)₁₋₃N(R^{a})₂,
(t) —(CH₂)₀₋₃C(═O)R^{t},
(u) —R^{t},
(v) —N(R^{a})R^{t}, and
(w) —(CH₂)₁₋₃R^{t}; and
R^{t} is selected from pyrrolidinyl, pyrazolidinyl, imidazolinyl, piperidinyl, piperazinyl, pyrrolyl, pyridyl, imidazolyl, pyrazolyl, triazolyl, tetrazolyl, pyrazinyl, pyrimidinyl, and pyradizinyl; any one of which is unsubstituted or substituted with 1 or 2 substituents independently selected from —F, —Cl, —Br, oxo, methyl, and methoxy;
   and all other variables are as defined in the twelfth embodiment;
   or a pharmaceutically acceptable salt thereof.

A fourth class of the present invention is a compound of Formula IV: 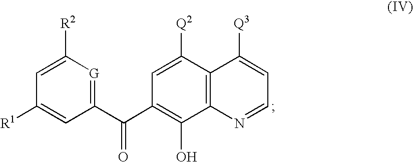
wherein G is N or CH;
and all other variables are as defined in the twelfth embodiment;
and provided that:

(i) when G is not N, Q² is H, and Q³ is H, then at least one of R¹ and R² is not —H;

(ii) when G is not N, Q² is halo or methyl or ethyl or phenyl optionally substituted with halo or —C₁₋₄ alkyl or benzyl optionally substituted with halo or —C₁₋₄ alkyl, and Q³ is H, then at least one of R₁ and R² is not —H, halo, methyl or ethyl; and

(iii) when G is not N, Q² is H, and Q³ is H, and one of R₁ and R² is —CO₂R^{a}, then the other of R₁ and R² is not —H;

or a pharmaceutically acceptable salt thereof.

A thirteenth embodiment of the present invention is a compound of Formula V: 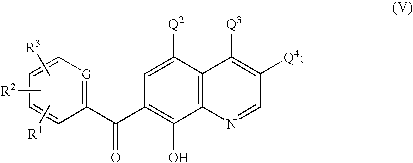
wherein G is N or is CH optionally substituted with one of R¹, R², and R³;
and all other variables are as originally defined;
and provided that when G is not N, Q² is OH, and Q³=Q⁴=H, then at least one of R¹, R², and R³ is not —H;
or a pharmaceutically acceptable salt thereof.

A fourteenth embodiment of the present invention is a compound of Formula (V), wherein

Q²is

(1) —H,

(2) methyl,

(3) ethyl,

(4) CF₃,

(5) —OH,

(6) methoxy,

(7) ethoxy

(8) —OCF₃

(9) halo selected from —F, —Cl and —Br,

(10) —CN,

(11) —CH₂OH,

(12) —CH₂OCH₃

(13) —SR^{a},

(14) —N(R^{a})₂,

(15) —SO₂R^{a},

(16) —C≡C—CH₂OR^{a}

(17) —N(R^{a})—(CH₂)₁₋₃SR^{a},

(18) —N(R^{a})—(CH₂)₁₋₃OR^{a},

(19) —N(R^{a})—(CH₂)₁₋₃N(R^{a})₂,

(20) —N(R^{a})—(CH₂)₁₋₃N(R^{a})—C(R^{a})═O,

(21) —R^{k},

(22) —(CH₂)₁₋₄R^{k},

(23) —C≡C—CH₂R^{k},

(24) —O—R^{k},

(25) —S—R^{k},

(26) —SO₂—R^{k},

(27) —N(R^{c})—R^{k},

(28) —N(R^{c})—(CH₂)₁₋₄R^{k},

(29) —N(R^{c})—(CH₂)₁₋₄OR^{k},

(30) —C(═O)N—(CH₂)₁₋₄R^{k},

(31) —C≡C—CH₂SR^{a}, or

(32) —C≡C—CH₂SO₂R^{a};

Q³ is —H or —C₁₋₄ alkyl;

Q⁴ is —H;

each of R¹ and R² is independently:

(1) —H,

(2) methyl,

(3) ethyl,

(4) CF₃,

(5) methoxy,

(6) ethoxy

(7) —OCF₃

(8) halo selected from —F, —Cl and —Br,

(9) —CN,

(10) —CH₂OR^{a},

(1 1) —CO₂R^{a},

(12) —SR^{a},

(13) —N(R^{a})₂,

(14) —(CH₂)₁₋₃N(R^{a})₂,

(15) —SO₂R^{a},

(16) —(CH₂)₁₋₂N(R^{a})—C(R^{a})═O,

(17) R^{k},

(18) —(CH₂)₁₋₄R^{k},

(19) —O—R^{k}, or

(20) —O—(CH₂)₁₋₄R^{k},

R³ is —H;

each R^{a} is independently —H or —C₁₋₄ alkyl;

each R^{c} is independently

(1) —H,

(2) —C₁₋₄ alkyl,

(3) —(CH₂)₁₋₄N(R^{a})₂, or

(4) —(CH₂)₁₋₄-phenyl, wherein the phenyl is optionally substituted with 1 to 3 substituents independently selected from halogen, C₁₋₄ alkyl, C₁₋₄ fluoroalkyl, —O—C₁₋₄ alkyl, —O—C₁₋₄ fluoroalkyl, —S—C₁₋₄ alkyl, —CN, and —OH; and

each R^{k} is independently:

(1) aryl selected from phenyl and naphthyl, wherein aryl is unsubstituted or substituted with from 1 to 4 substituents independently selected from:
(a) halogen,
(b) C₁₋₄ alkyl,
(c) C₁₋₄ fluoroalkyl,
(d) —O—C₁₋₄ alkyl,
(e) —O—C₁₋₄ fluoroalkyl,
(f) phenyl,
(g) —S—C₁₋₄ alkyl,
(h) —CN,
(i) —OH,
(j) phenyloxy, unsubstituted or substituted with from 1 to 3 substituents independently selected from:
   (i) halogen,
   (ii) C₁₋₄ alkyl,
   (iii) C₁₋₄ fluoroalkyl, and
   (iv) —OH,
(k) —N(R^{a})₂,
(l) —C₁₋₄ alkyl-N(R^{a})₂,
(m) —R^{t},
(p) —(CH₂)₀₋₃C(═O)N(R^{a})₂, and
(q) —(CH₂)₀₋₃C(═O)R^{a};

(2) —C₃₋₆ cycloalkyl, unsubstituted or substituted with from 1 to 3 substituents independently selected from:
(a) halogen,
(b) C₁₋₄ alkyl,
(c) —O—C₁₋₄ alkyl,
(d) C₁₋₄ fluoroalkyl,
(e) —O—C₁₋₄ fluoroalkyl,
(f) —CN,
(h) phenyl, and
(j) —OH;

(3) —C₃₋₆ cycloalkyl fused with a phenyl ring, unsubstituted or substituted with from 1 to 4 substituents independently selected from:
(a) halogen,
(b) C₁₋₄ alkyl,
(c) —O—C₁₋₄ alkyl,
(d) C₁₋₄ fluoroalkyl,
(e) —O—C₁₋₄ fluoroalkyl,
(f) —CN, and
(g) —OH;

(4) a 5- or 6-membered heteroaromatic ring selected from thienyl, pyridyl, imidazolyl, pyrrolyl, pyrazolyl, thiazolyl, isothiazolyl, oxazolyl, isooxazolyl, pyrazinyl, pyirimidinyl, triazolyl, tetrazolyl,furanyl, and pyridazinyl, wherein the heteroaromatic ring is unsubstituted or substituted on nitrogen or carbon with from 1 to 4 substituents independently selected from:
(a) halogen,
(b) C₁₋₄ alkyl,
(c) C₁₋₄fluoroalkyl,
(d) —O—C₁₋₄ alkyl,
(e) —O—C₁₋₄ fluoroalkyl,
(f) phenyl,
(g) —S—C₁₋₄ alkyl,
(h) —CN,
(i) —OH,
(j) phenyloxy, unsubstituted or substituted with from 1 to 3 substituents independently selected from:
   (i) halogen,
   (ii) C₁₋₄ alkyl,
   (iii) C₁₋₄ fluoroalkyl, and
   (iv) —OH,
(k) —N(R^{a})₂,
(l) —C₁₋₄ alkyl-N(R^{a})₂,
(m) R^{t},
(n) oxo,
(o) —(CH₂)₀₋₃C(═O)N(R^{a})₂, and
(p) —(CH₂)₀₋₃C(═O)R^{a};

(5) a 5- or 6- membered saturated heterocyclic ring selected from piperidinyl, morpholinyl, thiomorpholinyl, thiazolidinyl, isothiazolidinyl, oxazolidinyl, isooxazolidinyl, pyrrolidinyl, imidazolidinyl, piperazinyl, tetrahydrofuranyl, and pyrazolidinyl, wherein the heterocyclic ring is unsubstituted or substituted with from 1 to 3 substituents independently selected from:
(a) halogen,
(b) C₁₋₄ alkyl,
(c) —O—C₁₋₄ alkyl,
(d) C₁₋₄ fluoroalkyl,
(e) —O—C₁₋₄ fluoroalkyl,
(f) —CN,
(g) ═O,
((h) phenyl,
(i) benzyl,
(j) phenylethyl,
(k) —OH,
(l) —(CH₂)₀₋₃C(═O)N(R^{a})₂,
(m) —(CH₂)₀₋₃C(═O)R^{a},
(n) N(R^{a})—C(═O)R^{a},
(o) N(R^{a})—C(═O)OR^{a},
(p) (CH₂)₁₋₃N(R^{a})—C(═O)R^{a},
(q) N(R^{a})₂,
(r) (CH₂)₁₋₃N(R^{a})₂,
(s) —(CH₂)₀₋₃C(═O)R^{t},
(t) —R^{t},
(u) —N(R^{a})R^{t}, and
(v) —(CH₂)₁₋₃R^{t}; or

(6) an 8- to 10-membered heterobicyclic ring selected from indolyl, benzotriazolyl, benzoimidazolyl, imidazo[4,5-b]pyridinyl, dihydroimidazo[4,5-b]pyridinyl, pyrazolo[4,3-c]pyridinyl, dihydropyrazolo[4,3-c]pyridinyl, tetrahydropyrazolo[4,3-c]pyridinyl, pyrrolo[1,2-a]pyrazinyl, dihydropyrrolo[1,2-a]pyrazinyl, tetrahydropyrrolo[1,2-a]pyrazinyl, octahydropyrrolo[1,2-a]pyrazinyl, isoindolyl, indazolyl, indolinyl, isoindolinyl, quinolinyl, isoquinolinyl, quinoxalinyl, quinazolinyl, cinnolinyl, chromanyl, and isochromanyl, wherein the bicyclic ring is unsubstituted or substituted with 1 or 2 substituents independently selected from:
(a) halogen,
(b) C₁₋₄ alkyl,
(c) —O—C₁₋₄ alkyl,
(d) C₁₋₄ fluoroalkyl,
(e) —O—C₁₋₄ fluoroalkyl,
(f) —CN,
(g) ═O, and
(h) —OH;
   R^{t} is naphthyl or a 5- or 6-membered heteromonocylic ring selected from pyrrolidinyl, pyrazolidinyl, imidazolinyl, piperidinyl, piperazinyl, pyrrolyl, pyridyl, imidazolyl, pyrazolyl, triazolyl, tetrazolyl, pyrazinyl, pyrimidinyl, and pyradizinyl; and wherein the naphthyl or the heteromonocyclic ring is unsubstituted or substituted with 1 or 2 substituents independently selected from halogen, oxo, C₁₋₄ alkyl, and —O—C₁₋₄ alkyl;
   and provided that when G is not N, Q² is OH, and Q³ is H, then at least one of R¹ and R² is not —H;
   or a pharmaceutically acceptable salt thereof.

A fifth class of the present invention is a compound of Formula V,

wherein

R¹ is:

(1) —R^{k},

(2) —(CH₂)₁₋₄R^{k},

(3) —O—R^{k}, or

(4) —O—(CH₂)₁₋₄R^{k};

R² is:

(1) —H,

(2) methyl,

(3) ethyl,

(4) CF₃,

(5) methoxy,

(6) ethoxy

(7) —OCF₃

(8) halo selected from —F, —Cl and —Br,

(9) —CN,

(10) —CH₂OR^{a},

(11) —CO₂R^{a},

(12) —SR^{a},

(13) —N(R^{a})₂,

(14) —(CH₂)₁₋₃N(R^{a})₂,

(15) —SO₂R^{a},

(16) —(CH₂)₁₋₂N(R^{a})—C(R^{a})═O,

(17) —R^{k},

(18) —(CH₂)₁₋₄R^{k},

(19) —O—R^{k}, or

(20) —O—(CH₂)₁₋₄R^{k},

each R^{c} is independently —H or —C₁₋₄ alkyl;

each R^{k} is independently:

(1) phenyl which is unsubstituted or substituted with from 1 to 4 substituents independently selected from:
(a) halogen selected from —F, —Cl, and —Br,
(b) methyl,
(c) —CF₃,
(d) methoxy,
(e) —OCF₃,
(f) phenyl,
(g) —S—CH₃,
(h) —CN,
(i) —OH,
(j) phenyloxy, unsubstituted or substituted with from 1 to 3 substituents independently selected from:
   (i) halogen selected from —F, —Cl, and —Br,
   (ii) methyl,
   (iii) —CF₃, and
   (iv) —OH,
(k) —N(R^{a})₂,
(l) —(CH₂)₁₋₃N(R^{a})₂,
(m) R^{t},
(p) —(CH₂)₀₋₃C(═O)N(R^{a})₂, and
(q) —(CH₂)₀₋₃C(═O)R^{a};

(2) —C₃₋₆ cycloalkyl, unsubstituted or substituted with from 1 to 3 substituents independently selected from:
(a) halogen selected from —F, —Cl, and —Br,
(b) methyl,
(c) —CF₃,
(d) methoxy,
(e) —OCF₃,
(f) —CN,
(h) phenyl, and
(j) —OH;

(3) a 5- or 6- membered heteroaromatic ring selected from thienyl, pyridyl, imidazolyl, pyrrolyl, pyrazolyl, thiazolyl, isothiazolyl, oxazolyl, isooxazolyl, pyrazinyl, pyirimidinyl, triazolyl, tetrazolyl, furanyl, and pyridazinyl, wherein the heteroaromatic ring is unsubstituted or substituted on nitrogen or carbon with 1 or 2 substituents independently selected from:
(a) halogen selected from —F, —Cl, and —Br,
(b) methyl,
(c) —CF₃,
(d) methoxy,
(e) —OCF₃,
(f) phenyl,
(g) —S—C₁₋₆ alkyl,
(h) —CN,
(i) —OH,
(j) phenyloxy, unsubstituted or substituted with from 1 to 3 substituents independently selected from:
   (i) halogen selected from —F, —Cl, and —Br,
   (ii) methyl,
   (iii) —CF₃, and
   (iv) —OH,
(k) —N(R^{a})₂,
(l) —C₁₋₆ alkyl-N(R^{a})₂,
(m) R^{t},
(n) oxo,
(o) —(CH₂)₀₋₃C(═O)N(R^{a})₂, and
(p) —(CH₂)₀₋₃C(═O)R^{a}; and

(4) a 5- or 6-membered saturated heterocyclic ring selected from piperidinyl, morpholinyl, thiomorpholinyl, thiazolidinyl, isothiazolidinyl, oxazolidinyl, isooxazolidinyl, pyrrolidinyl, imidazolidinyl, piperazinyl, tetrahydrofuranyl, and pyrazolidinyl, wherein the heterocyclic ring is unsubstituted or, substituted with 1 or 2 substituents independently selected from:
(a) halogen selected from —F, —Cl, and —Br,
(b) methyl,
(c) —CF₃,
(d) methoxy,
(e) —OCF₃,
(f) —CN,
(g) ═O,
(h) phenyl,
(i) benzyl,
(j) phenylethyl,
(k) —OH,
(l) —(CH₂)₀₋₃C(═O)N(R^{a})₂,
(m) —(CH₂)₀₋₃C(═O)R^{a},
(n) N(R^{a})—C(═O)R^{a},
(o) N(R^{a})—C(═O)OR^{a},
(p) N(R^{a})—C(═O)OC(CH₃)₃,
(q) (CH₂)₁₋₃N(R^{a})—C(═O)R^{a},
(r) N(R^{a})₂,
(s) (CH₂)₁₋₃N(R^{a})₂,
(t) —(CH₂)₀₋₃C(═O)R^{t},
(u) —R^{t},
(v) —N(R^{a})R^{t}, and
(w) —(CH₂)₁₋₃R^{t}; and
   R^{t} is selected from pyrrolidinyl, pyrazolidinyl, imidazolinyl, piperidinyl, piperazinyl, pyrrolyl, pyridyl, imidazolyl, pyrazolyl, triazolyl, tetrazolyl, pyrazinyl, pyrimidinyl, and pyradizinyl; any one of which is unsubstituted or substituted with 1 or 2 substituents independently selected from —F, —Cl, —Br, oxo, methyl, and methoxy;
   and all other variables are as defined in the fourteenth embodiment;
   or a pharmaceutically acceptable salt thereof.

A sub-class of the present invention is a compound of Formula (VI): 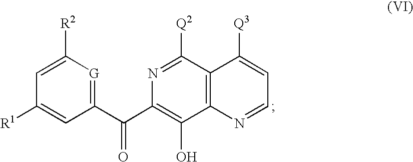
wherein G is N or CH;
and all other variables are as defined in the fifth class;
or a pharmaceutically acceptable salt thereof.

Exemplary compounds of the invention include compounds selected from the group consisting of:
1-(3-Benzylphenyl)-1-(8-hydroxyquinolin-7-yl)methanone;
1-(3-Benzylphenyl)-1-(8-hydroxy-4-methylquinolin-7-yl)methanone;
1-(3-Benzylphenyl)-1-(8-hydroxy-5-methylquinolin-7-yl)methanone;
1-[3-Benzyl-5-(1H-1,2,4-triazol-1-ylmethyl)phenyl]-1-(5-chloro-8-hydroxyquinolin-7-yl)methanone;
1-(3-Benzyl-5-imidazol-1-ylmethylphenyl)-1-(5-chloro-8-hydroxyquinolin-7-yl)methanone;
1-(4-Benzyl-pyridin-2-yl)-1-(8-hydroxyquinolin-7-yl)methanone;
1-(3-Benzylphenyl)-1-(8-hydroxy-[1,6]naphthyridin-7-yl)methanone;
1-[3-Benzyl-5-(1,1-dioxoisothiazolidin-2-ylmethyl)-phenyl]-1-(8-hydroxy-[1,6]naphthyridin-7-yl)methanone;
1-(3-Benzyl-5-(morpholin-4-ylmethyl)phenyl)- 1-(8-hydroxy-[1,6]naphthyridin-7-yl)methanone;
1-(3-Benzyl-5-piperidin-1-ylmethylphenyl)-1-(8-hydroxy-[1,6-naphthyridin-7-yl)methanone;
1-[3-Benzyl-5-(4-methylpiperazin-1-ylmethyl)phenyl]-1-(8-hydroxy-[1,6]naphthyridin-7-yl)methanone;
1-{3-Benzyl-5-[1-(8-hydroxy-[1 ,6]naphthyridin-7-yl)methanoyl]benzyl}-1H-pyridin-2-one;
3-{3-Benzyl-5-[(8-hydroxy-1,6-naphthyridin-7-yl)carbonyl]benzyl}-1-methylpyrimidine-2,4-(1H,3H)-dione;
1-[3-Benzyl-5-(tetrazol-1-ylmethyl)phenyl]-l -(8-hydroxy-[1,6]naphthyridin-7-yl)methanone;
1-[3-Benzyl-5-(tetrazol-2-ylmethyl)phenyl]-1-(8-hydroxy-[1,6]naphthyridin-7-yl)methanone;
1-(3-Benzyl-5-pyrazol-1-ylmethylphenyl)-1-(8-hydroxy-[1,6]naphthyridin-7-yl)methanone;
3-{3-Benzyl-5-[1-(8-hydroxy-[1,6]naphthyridin-7-yl)methanoyl]benzyl}-3H-pyrimidin-4-one;
1-{3-Benzyl-5-[1-(8-hydroxy-[1,6]naphthyridin-7-yl)methanoyl]benzyl}pyrrolidin-2-one;
N-{3-Benzyl-5-[1-(8-hydroxy-[1,6]naphthyridin-7-yl)methanoyl]benzyl}formamide;
N-{3-Benzyl-5-[1-(8-hydroxy-[1,6]naphthyridin-7-yl)methanoyl]benzyl}-N-methylformamide;
1-(8-hydroxy-[1,6]naphthyridin-7-yl)-1-(3-pyrazol-1-ylmethyl-5-pyridin-2-ylmethylphenyl)methanone;
1-(8-Hydroxy-[1,6]naphthyridin-7-yl)-1-[3-(1, 1 -dioxo-isothiazolidin-2-ylmethyl)-5-pyridin-2-ylmethylphenyl]methanone;
1-(8-Hydroxy-[1,6]naphthyridin-7-yl)-1-[3-(pyridin-2-one-1-ylmethyl)-5-pyridin-2-ylmethylphenyl]methanone;
1-(8-Hydroxy-[1,6]naphthyridin-7-yl)-1-[3-(piperidin-2-one- 1 -ylmethyl)-5-pyridin-2-ylmethylphenyl]methanone;
7-[1-(4-Benzylpyridin-2-yl)methanoyl]-8-hydroxy-6H-[1 ,6]naphthyridin-5-one;
   and pharmaceutically acceptable salts thereof.

Other embodiments of the present invention include the following:

(a) A pharmaceutical composition comprising a compound of Formula (I) and a pharmaceutically acceptable carrier.

(b) The pharmaceutical composition of (a), further comprising at least one antiviral selected from the group consisting of HIV protease inhibitors, non-nucleoside HIV reverse transcriptase inhibitors, and nucleoside HIV reverse transcriptase inhibitors.

(c) A method of inhibiting HIV integrase in a subject in need thereof which comprises administering to the subject a therapeutically effective amount of a compound of Formula (I).

(d) A method of preventing or treating infection by HIV in a subject in need thereof which comprises administering to the subject a therapeutically effective amount of a compound of Formula (I).

(e) The method of (d), wherein the compound of Formula (I) is administered in combination with a therapeutically effective amount of at least one antiviral selected from the group consisting of HIV protease inhibitors, non-nucleoside HIV reverse transcriptase inhibitors, and nucleoside HIV reverse transcriptase inhibitors.

(f) A method of preventing, treating or delaying the onset of AIDS in a subject in need thereof which comprises administering to the subject a therapeutically effective amount of a compound of Formula (I).

(g) The method of (f), wherein the compound is administered in combination with a therapeutically effective amount of at least one antiviral selected from the group consisting of HIV protease inhibitors, non-nucleoside HIV reverse transcriptase inhibitors, and nucleoside HIV reverse transcriptase inhibitors

(h) A method of inhibiting HIV integrase in a subject in need thereof which comprises administering to the subject a therapeutically effective amount of the composition of (a) or (b).

(i) A method of preventing or treating infection by HIV in a subject in need thereof which comprises administering to the subject a therapeutically effective amount of the composition of (a) or (b).

(j) A method of preventing, treating or delaying the onset of AIDS in a subject in need thereof which comprises administering to the subject a therapeutically effective amount of the composition of (a) or (b).

Still other embodiments of the present invention include the following:

(k) A pharmaceutical composition which comprises the product prepared by combining (e.g., mixing) an effective amount of a compound of Formula (I) and a pharmaceutically acceptable carrier.

(l) A combination useful for inhibiting HIV integrase, for treating or preventing infection by HIV, or for preventing, treating or delaying the onset of AIDS, which is a therapeutically effective amount of a compound of Formula (I) and a therapeutically effective amount of an HIV infection/AIDS treatment agent selected from the group consisting of HIV/AIDS antiviral agents, immunomodulators, and anti-infective agents.

(m) The combination of (1), wherein the HIV infection/AIDS treatment agent is an antiviral selected from the group consisting of HIV protease inhibitors, non-nucleoside HIV reverse transcriptase inhibitors and nucleoside HIV reverse transcriptase inhibitors.

Additional embodiments of the invention include the pharmaceutical compositions and methods set forth in (a)-(j) above and the compositions and combinations set forth in (k)-(m), wherein the compound employed therein is a compound of one of the embodiments, classes, sub-classes, or aspects of compounds described above. In all of these embodiments, the compound may optionally be used in the form of a pharmaceutically acceptable salt.

It is to be understood that the scope of the compounds of Formula (I) employed in the compositions, methods and combinations set forth above in (a)-(m) is limited only by the definitions of the variables in Formula I, and is not limited by any of the above provisos restricting the substitution on A when A is phenyl and X, Y and Z¹ to Z³ have certain values.

As used herein, the term “C₁₋₆ alkyl” (or “C₁-C₆ alkyl”) means linear or branched chain alkyl groups having from 1 to 6 carbon atoms and includes all of the hexyl alkyl and pentyl alkyl isomers as well as n-, iso-, sec- and t-butyl, n- and isopropyl, ethyl and methyl. “C₁₋₄ alkyl” means n-, iso-, sec- and t-butyl, n- and isopropyl, ethyl and methyl.

The term “C₀” as employed in expressions such as “C₀₋₆ alkyl” means a direct covalent bond.

The term “C₂₋₅ alkenyl” (or “C₂-C₅ alkenyl”) means linear or branched chain alkenyl groups having from 2 to 5 carbon atoms and includes all of the pentenyl isomers as well as 1-butenyl, 2-butenyl, 3-butenyl, isobutenyl, 1-propenyl, 2-propenyl, and ethenyl (or vinyl). Similar terms such as “C₂₋₃ alkenyl” have an analogous meaning.

The term “C₂₋₅ alkynyl” (or “C₂-C₅ alkynyl”) means linear or branched chain alkynyl groups having from 2 to 5 carbon atoms and includes all of the pentynyl isomers as well as 1-butynyl, 2-butynyl, 3-butynyl, 1-propynyl, 2-propynyl, and ethynyl (or acetylenyl). Similar terms such as “C₂₋₃ alkynyl” have an analogous meaning.

The term “C₃₋₇ cycloalkyl” (or “C₃-C₇ cycloalkyl”) means a cyclic ring of an alkane having three to seven total carbon atoms (i.e., cyclopropyl, cyclobutyl, cyclopentyl, cyclohexyl, or cycloheptyl). The term “C₃₋₆ cycloalkyl” refers to a cyclic ring selected from cyclopropyl, cyclobutyl, cyclopentyl, and cyclohexyl. Terms such as “C₃-C₅ cycloalkyl” have an analogous meaning.

The term “halogen” (or “halo”) refers to fluorine, chlorine, bromine and iodine (alternatively, fluoro, chloro, bromo, and iodo).

The term “thio” (also referred to as “thioxo”) means divalent sulfur; i.e., ═S.

The term “C₁₋₆ fluoroalkyl” (which may alternatively be referred to as “C₁-C₆ fluoroalkyl” or “fluorinated C₁-C₆ alkyl” or “C₁-C₆ fluoroalkyl”) means a C₁ to C₆ linear or branched alkyl group as defined above with one or more fluorine substituents. The term “fluorinated C₁-C₄ alkyl” has an analogous meaning. Representative examples of suitable fluoroalkyls include the series (CH₂)₀₋₄CF₃ (i.e., trifluoromethyl, 2,2,2-trifluoroethyl, 3,3,3-trifluoro-n-propyl, etc.), 1-fluoroethyl, 2-fluoroethyl, 2,2-difluoroethyl, 3,3,3-trifluoroisopropyl, 1,1,1,3,3,3-hexafluoroisopropyl, and perfluorohexyl.

The term “carbocycle” (and variations thereof such as “carbocyclic” or “carbocyclyl”) as used herein broadly refers to a C₃ to C₈ monocyclic, saturated or unsaturated ring or a C₇ to C₁₂ bicyclic ring system in which the rings are independent or fused and in which each ring is saturated or unsaturated. The carbocycle may be attached at any carbon atom which results in a stable compound. The fused bicyclic carbocycles are a subset of the carbocycles; i.e., the term “fused bicyclic carbocycle” generally refers to a C₇ to C₁₀ bicyclic ring system in which each ring is saturated or unsaturated and two adjacent carbon atoms are shared by each of the rings in the ring system. A subset of the fused bicyclic carbocycles are the fused bicyclic carbocycles in which one ring is a benzene ring and the other ring is saturated or unsaturated, with attachment via any carbon atom that results in a stable compound. Representative examples of this subset include the following:

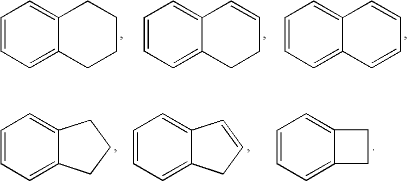

As used herein, the term “fused carbocyclic ring system” refers to a carbocycle as defined above which is fused to a phenyl ring. Representative examples include:

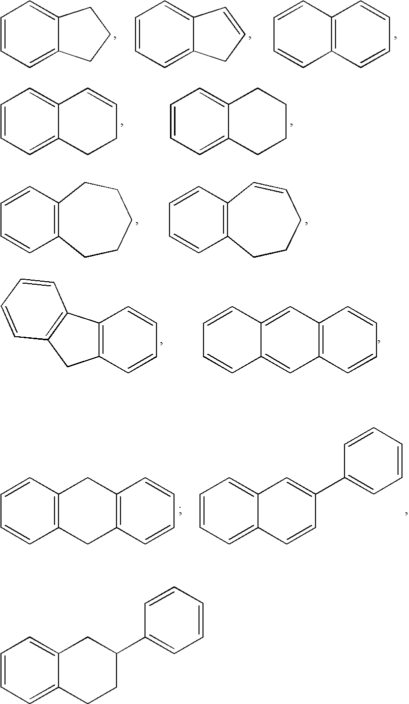

The term “aryl” refers to aromatic mono- and poly-carbocyclic ring systems, wherein the individual carbocyclic rings in the polyring systems may be fused or attached to each other via a single bond. Suitable aryl groups include, but are not limited to, phenyl, naphthyl, and biphenylenyl.

The term “heterocycle” (and variations thereof such as “heterocyclic” or “heterocyclyl”) broadly refers to a 4- to 8-membered monocyclic ring, 7- to 12-membered bicyclic ring system, or an 11 to 16-membered tricyclic ring system, any ring of which is saturated or unsaturated, and which consists of carbon atoms and one or more heteroatoms selected from N, 0 and S, and wherein the nitrogen and sulfur heteroatoms may optionally be oxidized, and the nitrogen heteroatom may optionally be quaternized. The heterocyclic ring may be attached at any heteroatom or carbon atom, provided that attachment results in the creation of a stable structure. When the heterocyclic ring has substituents, it is understood that the substituents may be attached to any atom in the ring, whether a heteroatom or a carbon atom, provided that a stable chemical structure results. Representative examples of heterocyclics include piperidinyl, piperazinyl, azepinyl, pyrrolyl, pyrrolidinyl, pyrazolyl, pyrazolidinyl, imidazolyl, imidazolidinyl, triazolyl, tetrazolyl, imidazolinyl, pyridyl (or pyridinyl), pyrazinyl, pyrimidinyl, pyridazinyl, oxazolyl, oxazolidinyl, isoxazolyl, isoxazolidinyl, morpholinyl, thiomorpholinyl, thiazolyl, thiazolidinyl, isothiazolyl, quinoxazolinyl, isothiazolidinyl, quinolinyl, isoquinolinyl, benzimidazolyl, thiadazolyl, benzopyranyl, benzothiazolyl, benzoazolyl, furyl (or furanyl), tetrahydrofuryl (or tetrahydrofuranyl), tetrahydropuranyl, thienyl (alternatively thiophenyl), benzothiophenyl, oxadiazolyl, and benzo-1,3-dioxacyclopentyl (alternatively, 1,3-benzodioxolyl). Representative examples of heterocyclics also include tetrahydrothienyl, tetrahydrodioxothienyl, thiadiazinanyl, dioxothiadiazinanyl, thiazinanyl, dioxothiazinanyl, dioxothiazolidinyl, and isodioxothiazolidinyl. Representative examples of heterocyclics also include the following bicyclics: indolyl, benzotriazolyl, imidazo[4,5-b]pyridinyl, dihydroimidazo[4,5-b]pyridinyl, pyrazolo[4,3-c]pyridinyl, dihydropyrazolo[4,3-c]pyridinyl, tetrahydropyrazolo[4,3-c]pyridinyl, pyrrolo[1,2-a]pyrazinyl, dihydropyrrolo[1,2-a]pyrazinyl, tetrahydropyrrolo[1,2-a]pyrazinyl, octahydropyrrolo[1,2-a]pyrazinyl, isoindolyl,

[symbol]

Representative examples of heterocyclics also include the following saturated monocyclics: hexahydropyrimidinyl, thiazinanyl (e.g., 1,2-thiazinanyl, alternatively named tetrahydro-1,2-thiazinyl), thiazepanyl (e.g., 1,4-thiazepanyl, alternatively named hexahydro-1,4-thiazepinyl), azepanyl (alternatively hexahydroazepinyl), thiadiazepanyl (e.g., 1,2,5-thiadiazepanyl), dithiazepanyl (e.g.,, 1,5,2,-dithiazepanyl), diazepanyl (e.g., 1,4-diazepanyl), and thiadiazinany] (e.g., 1,2,6-thiadiazinanyl).

A representative unsaturated heterocycle, optionally substituted, is 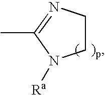
wherein p is an integer from zero to 4 and R^{a} is as defined above, and wherein each ring carbon is optionally and independently substituted with —C₁₋₄ alkyl.

Representative examples of heterocyclics also include the following bicyclics: hexahydropyrazolo[4,3-c]pyridinyl (e.g., 3a,4,5,6,7,7a-hexahydro-1H-pyrazolo[4,3c]pyridinyl), hexahydropurinyl (e.g., 2,3,4,5,6,7-hexahydro-1H-purinyl), hexahydrooxazolo[3,4a]pyrazinyl, and 1,2,3,4-tetrahydro-1,8-naphthyridinyl.

Fused ring heterocycles form a subset of the heterocycles as defined above; e.g., the term “fused bicyclic heterocycle” refers to a heteroatom-containing bicyclic ring system as defined in the preceding paragraph in which two adjacent atoms are shared by both rings. A subset of the fused bicyclic heterocycles is the fused bicyclic heterocycle containing carbon atoms and one or more heteroatoms selected from nitrogen, oxygen and sulfur, wherein one ring is a benzene ring and the other is a saturated or unsaturated heteroatom-containing ring. Representative examples of this subset include, but are not limited to, the following:

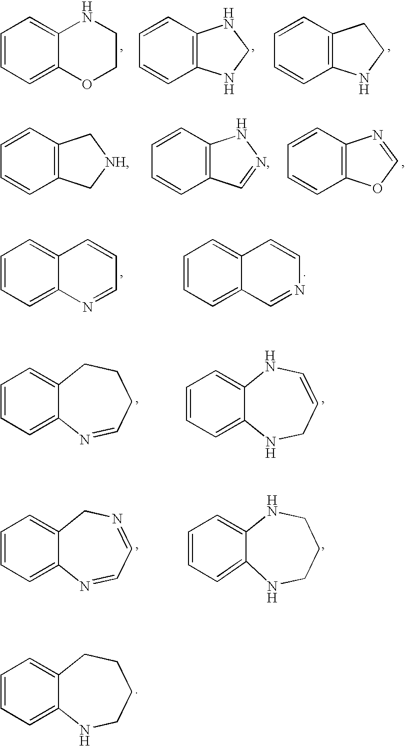

The term “heteromonocycle” (and variations thereof such as “heteromonocyclyl” or “heteromonocyclic”) refers to a 4- to 8-membered monocyclic ring which is saturated or unsaturated, and which consists of carbon atoms and one or more heteroatoms selected from N, O and S, and wherein the nitrogen and sulfur heteroatoms may optionally be oxidized, and the nitrogen heteroatom may optionally be quatemized. The heterocyclic ring may be attached at any heteroatom or carbon atom, provided that attachment results in the creation of a stable structure. Representative examples of monoheterocycles are disclosed above.

Heteroaromatics form another subset of the heterocycles as defined above; i.e., the term “heteroaromatic” (alternatively, “heteroaryl”) generally refers to a heterocycle as defined above in which the ring system (whether mono- or poly-cyclic) is an aromatic ring system. The term “heteroaromatic ring” refers to a monocyclic heterocycle as defined above which is an aromatic heterocycle. Representative examples of heteroaromatics include pyridyl, pyrrolyl, pyrazinyl, pyrimidinyl, pyridazinyl, thienyl (or thiophenyl), thiazolyl, furanyl, imidazolyl, pyrazolyl, triazolyl, tetrazolyl, oxazolyl, isooxazolyl, oxadiazolyl, thiazolyl, isothiazolyl, and thiadiazolyl.

Unless expressly set forth to the contrary, an “unsaturated” ring is a partially or fully unsaturated ring. For example, an “unsaturated monocyclic C₆ carbocycle” refers to cyclohexene, cyclohexadine, and benzene.

The present invention includes pharmaceutical compositions useful for inhibiting EV integrase, comprising an effective amount of a compound of this invention, and a pharmaceutically acceptable carrier. Pharmaceutical compositions useful for treating infection by HIV, or for treating AIDS or ARC, are also encompassed by the present invention, as well as a method of inhibiting HIV integrase, and a method of treating infection by HIV, or of treating ADS or ARC. Additionally, the present invention is directed to a pharmaceutical composition comprising a therapeutically effective amount of a compound of the present invention in combination with a therapeutically effective amount of an agent for treating HIV infection or AIDS selected from:

(1) an antiviral agent useful for treating or preventing HIV infection or for treating AIDS (also referred to herein as an HIV/AIDS antiviral agent),

(2) an anti-infective agent, and

(3) an immunomodulator.

The present invention also includes a compound of the present invention for use in (a) inhibiting HIV protease, (b) preventing or treating infection by HIV, or (c) preventing, treating or delaying the onset of AIDS or ARC. The present invention also includes the use of a compound of the present invention as described above as a medicament for (a) inhibiting HIV integrase, (b) preventing or treating infection by HIV, or (c) preventing, treating or delaying the onset of AIDS or ARC. The present invention further includes the use of any of the HIV integrase inhibiting compounds of the present invention as described above in combination with one or more HIV/AIDS treatment agents selected from an HIV/AIDS antiviral agent, an anti-infective agent, and an immunomodulator as a medicament for (a) inhibiting HIV integrase, (b) preventing or treating infection by HIV, or (c) preventing, treating or delaying the onset of AIDS or ARC, said medicament comprising an effective amount of the HIV integrase inhibitor compound and an effective amount of the one or more treatment agents.

The present invention also includes the use of a compound of the present invention as described above in the preparation of a medicament for (a) inhibiting HIV integrase, (b) preventing or treating infection by HIV, or (c) preventing, treating or delaying the onset of AIDS or ARC.

The present invention further includes the use of any of the HIV integrase inhibiting compounds of the present invention as described above in combination with one or more HIV/AIDS treatment agents selected from an HIV/AIDS antiviral agent, an anti-infective agent, and an immunomodulator for the manufacture of a medicament for (a) inhibiting HIV integrase, (b) preventing or treating infection by HIV, or (c) preventing, treating or delaying the onset of AIDS or ARC, said medicament comprising an effective amount of the HIV integrase inhibitor compound and an effective amount of the one or more treatment agents.

The compounds of the present invention may have asymmetric centers and may occur, except when specifically noted, as mixtures of stereoisomers or as individual diastereomers, or enantiomers, with all isomeric forms being included in the present invention.

As is recognized by one of ordinary skill in the art, certain of the compounds of the present invention (e.g., the 5,8-dihydroxy-1,6-naphthyridin-7-yl methanone compounds and the 4,8-dihydroxy-1,6-naphthyridin-7-yl methanone compounds) can exist as tautomers: 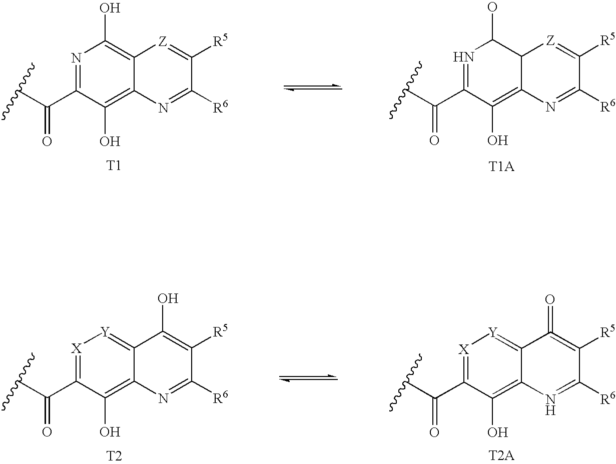
It is to be understood for the purposes of the present invention that a reference herein to a compound of Formula T1 is a reference to compound T1 per se, its tautomer T1A per se, or mixtures thereof. Likewise, a reference to a compound of Formula T2 is a reference to compound T2 per se, its tautomer T2A per se, or mixtures thereof.

When any variable (e.g., R^{a}, R^{b}, R^{c}, R^{k}, etc.) occurs more than one time in any constituent or in Formula I or in any other formula depicting and describing compounds of the invention, its definition on each occurrence is independent of its definition at every other occurrence. Also, combinations of substituents and/or variables are permissible only if such combinations result in stable compounds.

The term “substituted” (e.g., as in “phenyl ring, unsubstituted or substituted with from 1 to 5 substituents . . .”) includes mono- and poly-substitution by a named substituent to the extent such single and multiple substitution is chemically allowed. For example, a carbocycle or heterocycle substituted with more than one substituent can have multiple substituents on the same ring atom to the extent it is chemically permitted. A ring sulfur atom in a saturated heterocycle can, for example, typically be substituted with 1 ( —S(═O)—) or 2 oxo groups (—SO₂—).

The compounds of the present inventions are useful in the inhibition of HIV integrase, the prevention or treatment of infection by human immunodeficiency virus (HIV) and the treatment of consequent pathological conditions such as AIDS. Treating AIDS or preventing or treating infection by HIV is defined as including, but not limited to, treating a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by e.g., blood transfusion, exchange of body fluids, bites, accidental needle stick, or exposure to patient blood during surgery.

The compounds of this invention are useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV integrase, e.g., by competitive inhibition. Thus the compounds of this invention are commercial products to be sold for these purposes.

The present invention also provides for the use of a compound of Formula (I) to make a pharmaceutical composition useful for inhibiting HIV integrase and in the treatment of AIDS or ARC.

The compounds of the present invention may be administered in the form of pharmaceutically acceptable salts. The term “pharmaceutically acceptable salt” is intended to include all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. Depending on the particular functionality of the compound of the present invention, pharmaceutically acceptable salts of the compounds of this invention include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide. These salts may be prepared by standard procedures, e.g. by reacting a free acid with a suitable organic or inorganic base. Where a basic group is present, such as amino, an acidic salt, i.e. hydrochloride, hydrobromide, acetate, pamoate, and the like, can be used as the dosage form.

Also, in the case of an acid (—COOH) or alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

For these purposes, the compounds of the present invention may be administered orally, parenterally (including subcutaneous injections, intravenous, intramuscular, intrasternal injection or infusion techniques), by inhalation spray, or rectally, in dosage unit formulations containing conventional non-toxic pharmaceutically-acceptable carriers, adjuvants and vehicles.

The term “administration” and variants thereof (e.g., “administering” a compound) in reference to a compound of the invention each mean providing the compound or a prodrug of the compound to the individual in need of treatment. When a compound of the invention or prodrug thereof is provided in combination with one or more other active agents (e.g., antiviral agents useful for treating HIV infection or AIDS), “administration” and its variants are each understood to include concurrent and sequential provision of the compound or prodrug thereof and other agents.

Thus, in accordance with the present invention there is further provided a method of treating and a pharmaceutical composition for treating HIV infection and AIDS. The treatment involves administering to a subject in need of such treatment a pharmaceutical composition comprising a pharmaceutical carrier and a therapeutically-effective amount of a compound of the present invention.

As used herein, the term “composition” is intended to encompass a product comprising the specified ingredients in the specified amounts, as well as any product which results, directly or indirectly, from combination of the specified ingredients in the specified amounts.

By “pharmaceutically acceptable” it is meant the carrier, diluent or excipient must be compatible with the other ingredients of the formulation and not deleterious to the recipient thereof.

The term “subject,” (alternatively referred to herein as “patient”) as used herein refers to an animal, preferably a mammal, most preferably a human, who has been the object of treatment, observation or experiment.

The term “therapeutically effective amount” as used herein means that amount of active compound or pharmaceutical agent that elicits the biological or medicinal response in a tissue, system, animal or human that is being sought by a researcher, veterinarian, medical doctor or other clinician, which includes alleviation of the symptoms of the disease being treated.

These pharmaceutical compositions may be in the form of orally-administrable suspensions or tablets or capsules, nasal sprays, sterile injectible preparations, for example, as sterile injectible aqueous or oleagenous suspensions or suppositories.

When administered orally as a suspension, these compositions are prepared according to techniques well-known in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The injectible solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

The compounds of this invention can be administered orally to humans in a dosage range of 0.1 to 1000 mg/kg body weight in divided doses. One preferred dosage range is 0.1 to 200 mg/kg body weight orally in divided doses. Another preferred dosage range is 0.5 to 100 mg/kg body weight orally in divided doses. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The present invention is also directed to combinations of the HIV integrase inhibitor compounds with one or more agents useful in the treatment of HIV infection or AIDS. For example, the compounds of this invention may be effectively administered, whether at periods of pre-exposure and/or post-exposure, in combination with effective amounts of the HIV/AIDS antivirals, imunomodulators, antiinfectives, or vaccines useful for treating HIV infection or AIDS, such as those in the following Table.

| | | |
|---|---|---|
| Drug Name | Manufacturer | Indication |

ANTIVIRALS

| | | |
|---|---|---|
| Amprenavir | Glaxo Wellcome | HIV infection, AIDS, |
| 141 W94 | | ARC |
| GW 141 | | (protease inhibitor) |
| Abacavir | Glaxo Welcome | HIV infection, AIDS, |
| GW 1592 | | ARC |
| 1592U89 | | (reverse transcriptase |
| | | inhibitor) |
| Acemannan | Carrington Labs | ARC |
| | (Irving, TX) | |
| Acyclovir | Burroughs Wellcome | HIV infection, AIDS, ARC, |
| | | in combination with AZT |
| AD-439 | Tanox Biosystems | HIV infection, AIDS, ARC |
| AD-519 | Tanox Biosystems | HIV infection, AIDS, ARC |
| Adefovir dipivoxil | Gilead Sciences | HIV infection |
| AL-721 | Ethigen | ARC, PGL, HIV positive, |
| | (Los Angeles, CA) | AIDS |
| Alpha Interferon | Glaxo Wellcome | Kaposi's sarcoma, HIV, in |
| | | combination w/Retrovir |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| neutralizes pH | Concepts | |
| labile alpha aberrant | (Rockville, MD) | |
| Interferon | | |
| AR177 | Aronex Pharm | HIV infection, AIDS, ARC |
| beta-fluoro-ddA | Nat'l Cancer Institute | AIDS-associated diseases |
| BMS-232623 | Bristol-Myers Squibb/ | HIV infection, AIDS, |
| (CGP-73547) | Novartis | ARC |
| | | (protease inhibitor) |
| BMS-234475 | Bristol-Myers Squibb/ | HIV infection, AIDS, |
| (CGP-61755) | Novartis | ARC |
| | | (protease inhibitor) |
| CI-1012 | Warner-Lambert | HIV-1 infection |
| Cidofovir | Gilead Science | CMV retinitis, herpes, |
| | | papillomavirus |
| Curdlan sulfate | AJI Pharma USA | HIV infection |
| Cytomegalovirus immune | MedImmune | CMV retinitis |
| globin | | |
| Cytovene | Syntex | sight threatening CMV |
| Ganciclovir | | peripheral CMV |
| | | retinitis |
| Delaviridine | Pharmacia-Upjohn | HIV infection, AIDS, |
| | | ARC |
| | | (protease inhibitor) |
| Dextran Sulfate | Ueno Fine Chem. | AIDS, ARC, HIV |
| | Ind. Ltd. (Osaka, Japan) | positive asymptomatic |
| ddC | Hoffman-La Roche | HIV infection, AIDS, ARC |
| Dideoxycytidine | | |
| ddI | Bristol-Myers Squibb | HIV infection, AIDS, ARC; |
| Dideoxyinosine | | combination with AZT/d4T |
| mozenavir | AVID | HIV infection, AIDS, |
| (DMP-450) | (Camden, NJ) | ARC |
| | | (protease inhibitor) |
| EL10 | Elan Corp, PLC | HIV infection |
| | (Gainesville, GA) | |
| Efavirenz | DuPont (SUSTIVA ®), | HIV infection, AIDS, |
| (DMP 266) | Merck (STOCRIN ®) | ARC |
| (-)6-Chloro-4(S)- | | (non-nucleoside RT |
| cyclopropylethynyl- | | inhibitor) |
| 4(S)-trifluoro-methyl- | | |
| 1,4-dihydro-2H-3,1- | | |
| benzoxazin-2-one, | | |
| Famciclovir | Smith Kline | herpes zoster, herpes |
| | | simplex |
| FTC | Emory University | HIV infection, AIDS, ARC |
| | | (reverse transcriptase |
| | | inhibitor) |
| GS 840 | Gilead | HIV infection, AIDS, ARC |
| | | (reverse transcriptase |
| | | inhibitor) |
| HBY097 | Hoechst Marion Roussel | HIV infection, AIDS, ARC |
| | | (non-nucleoside reverse |
| | | transcriptase inhibitor) |
| Hypericin | VIMRx Pharm. | HIV infection, AIDS, ARC |
| Recombinant Human | Triton Biosciences | AIDS, Kaposi's sarcoma, |
| Interferon Beta | (Almeda, CA) | ARC |
| Interferon alfa-n3 | Interferon Sciences | ARC, AIDS |
| Indinavir | Merck | HIV infection, AIDS, ARC, |
| | | asymptomatic HIV positive, |
| | | also in combination with |
| | | AZT/ddI/ddC |
| Compound A | Merck | HIV infection, AIDS, |
| | | ARC, asymptomatic HIV |
| | | positive |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, AIDS, |
| | | ARC (reverse |
| | | transcriptase inhibitor); |
| | | also with AZT |
| Lobucavir | Bristol-Myers Squibb | CMV infection |
| Nelfinavir | Agouron | HIV infection, AIDS, |
| | Pharmaceuticals | ARC (protease inhibitor) |
| Nevirapine | Boeheringer | HIV infection, AIDS, |
| | Ingleheim | ARC (protease inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV infection, |
| Phosphonoformate | Products, Inc | other CMV |
| | | infections |
| PNU-140690 | Pharmacia Upjohn | HIV infection, AIDS, ARC |
| | | (protease inhibitor) |
| Probucol | Vyrex | HIV infection, AIDS |
| RBC-CD4 | Sheffield Med. Tech | HIV infection, AIDS, |
| | (Houston TX) | ARC |
| Ritonavir | Abbott | HIV infection, AIDS, |
| (ABT-538) | | ARC (protease inhibitor) |
| Saquinavir | Hoffmann-LaRoche | HIV infection, AIDS, |
| | | ARC (protease inhibitor) |
| Stavudine; d4T | Bristol-Myers Squibb | HIV infection, AIDS, ARC |
| Didehydrodeoxy- | | |
| thymidine | | |
| Valaciclovir | Glaxo Wellcome | genital HSV & CMV |
| | | infections |
| Virazole | Viratek/ICN | asymptomatic HIV |
| Ribavirin | (Costa Mesa, CA) | positive, LAS, ARC |
| VX-478 | Vertex | HIV infection, AIDS, ARC |
| Zalcitabine | Hoffmann-La Roche | HIV infection, AIDS, ARC, |
| | | with AZT |
| Zidovudine; AZT | Glaxo Wellcome | HIV infection, AIDS, ARC, |
| | | Kaposi's sarcoma in |
| | | combination with other |
| | | therapies (reverse |
| | | transcriptase inhibitor) |
| ABT-378; Lopinavir | Abbott | HIV infection, AIDS, ARC |
| | | (protease inhibitor) |
| ABT-378/r; contains | Abbott | HIV infection, AIDS, ARC |
| lopinavir and ritonavir; | | (protease inhibitor) |
| Kaletra | | |
| JE2147/AG1776 | Agouron | HIV infection, AIDS, ARC |
| | | (protease inhibitor) |
| T-20 | Trimeris | HIV infection, AIDS, ARC |
| | | (fusion inhibitor) |
| T-1249 | Trimeris | HIV infection, AIDS, ARC |
| | | (fusion inhibitor) |
| atazanavir | Bristol-Myers-Squibb | HIV infection, AIDS, ARC |
| (BMS 232632) | | (protease inhibitor) |
| PRO 542 | Progenics | HIV infection, AIDS, ARC |
| | | (attachment inhibitor) |
| PRO 140 | Progenics | HIV infection, AIDS, ARC |
| | | (CCR5 co-receptor inhibitor) |
| TAK-779 | Takeda | HIV infection, AIDS, ARC |
| | | (injectable CCR5 receptor |
| | | antagonist) |
| DPC 681 & DPC 684 | DuPont | HIV infection, AIDS, ARC |
| | | (protease inhibitors) |
| DPC 961 & DPC 083 | DuPont | HIV infection AIDS, ARC |
| | | (nonnucleoside reverse |
| | | transcriptase inhibitors) |
| Trizivir (contains abacavir, | GlaxoSmithKline | HIV infection, AIDS, ARC |
| lamivudine, and | | (reverse transcriptase |
| zidovudine) | | inhibitors) |
| tipranavir (PNU-140690) | Boehringer Ingelheim | HIV infection, AIDS, ARC |
| | (purchased from | (protease inhibitor) |
| | Pharmacia & Upjohn) | |
| tenofovir disoproxil | Gilead | HIV infection, AIDS, ARC |
| fumarate | | (reverse transcriptase |
| | | inhibitor) |
| TMC-120 & TMC-125 | Tibotec | HIV infections, AIDS, ARC |
| | | (non-nucleoside reverse |
| | | transcriptase inhibitors) |
| TMC-126 | Tibotec | HIV infection, AIDS, ARC |
| | | (protease inhibitor) |

IMMUNO-MODULATORS

| | | |
|---|---|---|
| AS-101 | Wyeth-Ayerst | AIDS |
| Bropirimine | Pharmacia Upjohn | advanced AIDS |
| Acemannan | Carrington Labs, Inc. | AIDS, ARC |
| | (Irving, TX) | |
| CL246, 738 | American Cyanamid | AIDS, Kaposi's sarcoma |
| | Lederle Labs | |
| EL10 | Elan Corp, PLC | HIV infection |
| | (Gainesville, GA) | |
| FP-21399 | Fuki ImmunoPharm | blocks HIV fusion with |
| | | CD4+ cells |
| Gamma Interferon | Genentech | ARC, in combination w/TNF |
| | | (tumor necrosis factor) |
| Granulocyte | Genetics Institute | AIDS |
| Macrophage Colony | Sandoz | |
| Stimulating | | |
| Factor | | |
| Granulocyte | Hoeschst-Roussel | AIDS |
| Macrophage Colony | Immunex | |
| Stimulating | | |
| Factor | | |
| Granulocyte | Schering-Plough | AIDS, combination w/AZT |
| Macrophage Colony | | |
| Stimulating Factor | | |
| HIV Core Particle | Rorer | seropositive HIV |
| Immunostimulant | | |
| IL-2 | Cetus | AIDS, in combination |
| Interleukin-2 | | w/AZT |
| IL-2 | Hoffman-La Roche | AIDS, ARC, HIV, in |
| Interleukin-2 | Immunex | combination w/AZT |
| IL-2 | Chiron | AIDS, increase in CD4 cell |
| Interleukin-2 | | counts |
| (aldeslukin) | | |
| Immune Globulin | Cutter Biological | pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's sarcoma, |
| | (New Orleans, LA) | ARC, PGL |
| Imuthiol Diethyl | Merieux Institute | AIDS, ARC |
| Dithio Carbamate | | |
| Alpha-2 | Schering Plough | Kaposi's sarcoma w/AZT, |
| Interferon | | AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte | Amgen | AIDS, in combination |
| Colony Stimulating | | w/AZT |
| Factor | | |
| Remune | Immune Response Corp. | immunotherapeutic |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma, AIDS, |
| Alfa 2a | | ARC, in combination w/AZT |
| SK&F106528 | Smith Kline | HIV infection |
| Soluble T4 | | |
| Thymopentin | Immunobiology | HIV infection |
| | Research Institute | |
| Tumor Necrosis | Genentech | ARC, in combination |
| Factor; TNF | | w/gamma Interferon |
| etanercept | Immunex Corp | rheumatoid arthritis |
| | (Enbrel ®) | |
| infliximab | Centocor (Remicade ®) | rheumatoid arthritis and |
| | | Crohn's disease |

ANTI-INFECTIVES

| | | |
|---|---|---|
| Clindamycin with | Pharmacia Upjohn | PCP |
| Primaquine | | |
| Fluconazole | Pfizer | cryptococcal meningitis, |
| | | candidiasis |
| Pastille | Squibb Corp. | prevention of oral candidiasis |
| Nystatin Pastille | | |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | antibacterial |
| Trimethoprim/sulfa | | antibacterial |
| Piritrexim | Burroughs Wellcome | PCP treatment |
| Pentamidine | Fisons Corporation | PCP prophylaxis |
| isethionate for | | |
| inhalation | | |
| Spiramycin | Rhone-Poulenc | cryptosporidia diarrhea |
| Intraconazole- | Janssen Pharm. | histoplasmosis; cryptococcal |
| R51211 | | meningitis |
| Trimetrexate | Warner-Lambert | PCP |

OTHER

| | | |
|---|---|---|
| Daunorubicin | NeXstar, Sequus | Karposi's sarcoma |
| Recombinant Human | Ortho Pharm. Corp. | severe anemia assoc. with |
| Erythropoietin | | AZT therapy |
| Recombinant Human | Serono | AIDS-related wasting, |
| Growth Hormone | | cachexia |
| Leukotriene B4 Receptor | — | HIV infection |
| Antagonist | | |
| Megestrol Acetate | Bristol-Myers Squibb | treatment of anorexia assoc. |
| | | w/AIDS |
| Soluble CD4 Protein and | — | HIV infection |
| Derivatives | | |
| Testosterone | Alza, Smith Kline | AIDS-related wasting |
| Total Enteral | Norwich Eaton | diarrhea and malabsorption, |
| Nutrition | Pharmaceuticals | related to AIDS |

It will be understood that the scope of combinations of the compounds of this invention with HIV/AIDS antivirals, immunomodulators, anti-infectives or vaccines is not limited to the list in the above Table, but includes in principle any combination with any pharmaceutical composition useful for the treatment of HIV infection or AIDS. When employed in combination with the compounds of the invention, the HIV/AIDS antivirals and other agents are typically employed in their conventional dosage ranges and regimens as reported in the art, including the dosages described in the Physicians' Desk Reference, 54^{th} edition, Medical Economics Company, 2000. The dosage ranges for a compound of the invention in these combinations are the same as those set forth above just before the Table.

Preferred combinations are simultaneous or sequential treatments of a compound of the present invention and an inhibitor of HIV protease and/or a non-nucleoside inhibitor of HIV reverse transcriptase. An optional fourth component in the combination is a nucleoside inhibitor of HIV reverse transcriptase, such as AZT, 3TC, ddC or ddl. A preferred inhibitor of HIV protease is the sulfate salt of indinavir, which is N-(2(R)-hydroxy- 1 (S)-indanyl)-2(R)-phenylmethyl-4-(S)-hydroxy-5-(1-(4-(3-pyridyl-methyl)-2(S)-N′-(t-butylcarboxamido)-piperazinyl))-pentaneamide ethanolate, and is synthesized according to U.S. Pat. No. 5413999. Indinavir is generally administered at a dosage of 800 mg three times a day. Other preferred protease inhibitors are nelfinavir and ritonavit. Another preferred inhibitor of HIV protease is saquinavir which is administered in a dosage of 600 or 1200 mg tid. Still another preferred protease inhibitor is Compound A, which is N-(2(R)-hydroxy-1(S)-indanyl)-2(R)-phenylmethyl-4(S)-hydroxy-5-(1-(4-(2-benzo[b]furanylmethyl)-2(S)-N′-(t-butylcarboxamido)piperazinyl))pentaneamide, preferably administered as the sulfate salt. Compound A can be prepared as described in U.S. Pat. No. 5,646,148. Preferred non-nucleoside inhibitors of HIV reverse transcriptase include efavirenz. The preparation of ddC, ddI and AZT are also described in EPO 0,484,071. These combinations may have unexpected effects on limiting the spread and degree of infection of HIV. Preferred combinations include a compound of the present invention with the following (1) indinavir with efavirenz, and, optionally, AZT and/or 3TC and/or ddI and/or ddC; (2) indinavir, and any of AZT and/or ddI and/or ddC and/or 3TC, in particular, indinavir and AZT and 3TC; (3) stavudine and 3TC and/or zidovudine; (4) zidovudine and lamivudine and 141W94 and 1592U89; (5) zidovudine and lamivudine.

Another preferred combination is a compound of the present invention with indinavir and Compound A and optionally with one or more of efavirenz, AZT, 3TC, ddl and ddC. In one embodiment of this combination, the weight ratio of indinavir to Compound A is from about 1:1 to about 1:2, wherein the amount of indinavir employed is in the range of from about 200 to about 1000 mg. Indinavir and Compound A can be administered concurrently or sequentially in either order from one to three times per day.

In such combinations the compound of the present invention and other active agents may be administered together or separately. In addition, the administration of one agent may be prior to, concurrent to, or subsequent to the administration of other agent(s).

Abbreviations used in the instant specification, particularly the Schemes and Examples, are as follows:

Ac=acetyl

Et=ethyl

EtOAc=ethyl acetate

Bu=butyl

n-BuLi=n-butyl lithium

DMF=N,N-dimethylformamide

DMSO=dimethylsulfoxide

ES MS=electrospray mass spectrometry

Et₃N=triethylamine

EtOH=ethanol

HPLC=high performance liquid chromatography

Me=methyl

MeOH=methanol

NMR=nuclear magnetic resonance

rt and RT=room temperature

TFA=trifluoroacetic acid

THF=tetrahydrofuran

The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above.

Scheme 1 presents a general method for preparing 8-hydroxyquinoline derivatives, wherein 7-halo-8-alkoxyquinoline 1-1 can be treated with alkyllithium, followed by coupling of the lithiated 1-1 with carboxylic derivative 1-2 to provide ketone 1-3 of the present invention. Removal of the 8-hydroxy protecting group (e.g., by treating with TFA) provided the required 8-hydroxyquinoline ketones. The 7-halo-8-alkoyquinoline 1-1 can also be coupled with an aldehyde 1-4 and then deprotected to provide alcohol 1-5, which can then be oxidized to afford ketone 1-3.

The starting quinolines of formula 1-1 can be prepared via methods described in Pearson et al., J. Org. Chem. 1967, 32: 2358-2360, or routine variations thereof. The starting carboxylic derivatives of formula 1-2 can be prepared via methods described in Budesinsky et al., Magn.Reson.Chem. 1989, 27: 585-591; or routine variations thereof.

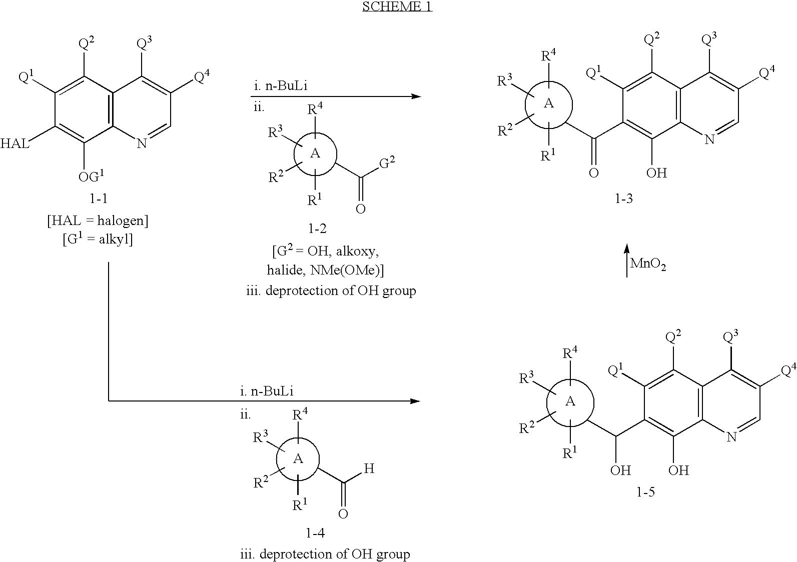

An alternative general approach is set forth in Scheme 2, wherein an appropriate aryl or heterocyclyl halide 2-1 can be lithiated and coupled with an 8-alkoxyquinoline-7-carboxylic derivative 2-2 to provide quinolinyl compounds of the present invention. The starting halides of formula 2-1 can be prepared via methods described in Mechelkeet al., J. Org. Chem. 1999, 64: 4821-4829; or routine variations thereof. The starting alkoxyquinoline carboxylic esters of formula 2-2 can be prepared via methods described in Belser et al. Tetrahedron 1996, 52: 2937-2944 and Baret et al., J. Am. Chem. Soc. 1995, 117: 9760-9761; or routine variations thereof.

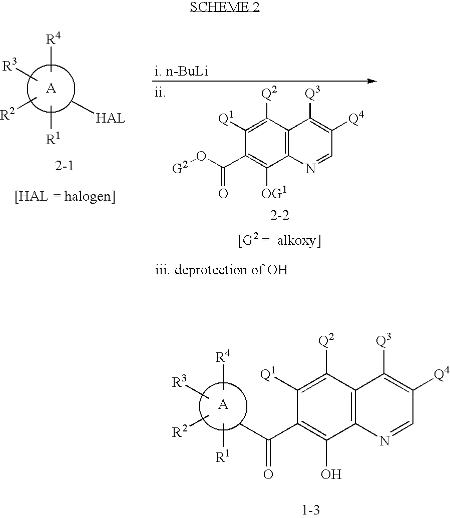

A general approach for preparing (8-hydroxy-[1,6]naphthyridin-7-yl)methanones is shown in Scheme 3, wherein an adduct of bromoketone 3-2 and 3-aminomethyl-2-chloropyridine 3-3 can be treated with either CBz chloride or benzenesulfonyl chloride. The resulting product 3-4 can be alkoxycarbonylated to give compound 3-5, and then treated with sodium alkoxide to provide the appropriately substituted napthyridine 3-7 from the benzenesulfonyl derivative directly and from the CBZ derivative via a CBZ removal step and then an oxidation step.

The 3-aminomethyl-2-chloropyridines of formula 3-3 can be prepared via via 3-hydroxymethyl-2-chloropyridines as described in Read et al., J. Het. Chem. 1995, 32: 1595, or routine variations thereof. 3-Hydroxymethyl-2-chloropyridines can then be transformed to the corresponding aminomethyl derivatives via the corresponding chloromethyl and azidomethyl derivatives.

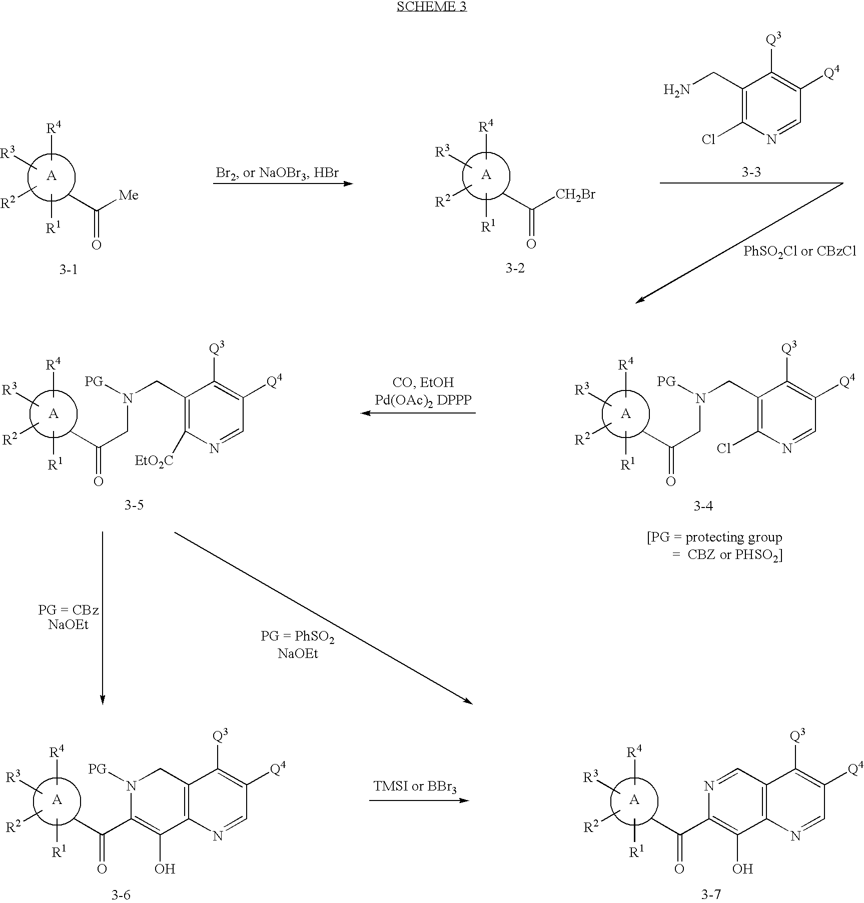

A general approach for preparing of 8-hydroxy-6H-[1,6]naphthyridin-5-ones is presented in Scheme 4. The coupling product 4-2 of bromoketone 3-2 and pyrrolo[3,4-b]pyridine-5,7-dione 4-1 can be treated with sodium alkoxide to provide a mixture of regioisomers 4-3 and 4-4, which can be separated by conventional methods (e.g., HPLC). Chemistry related to that set forth in Scheme 4 is described in M. Blanco et al., J. Heterocycl. Chem. 1996, 33: 361-366.

The pyrrolopyridinediones of formula 4-1 can be prepared via methods described in U.S. Pat. No. 3,887,550, or routine variations thereof.

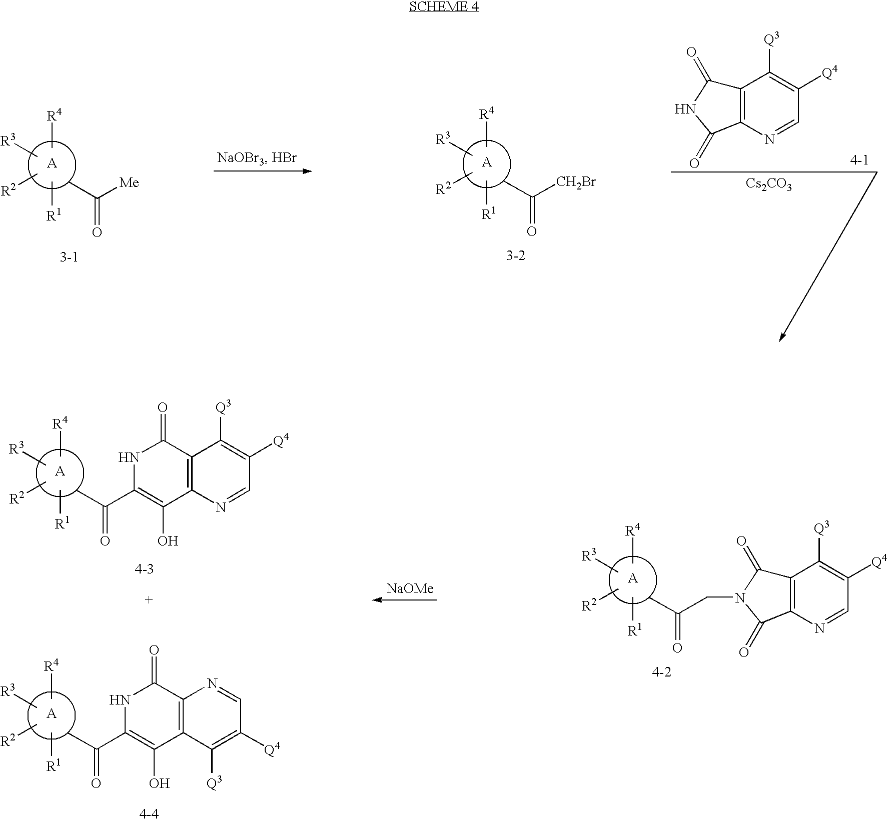

In the processes for preparing compounds of the present invention set forth in the foregoing schemes, functional groups in various moieties and substituents may be sensitive or reactive under the reaction conditions employed and/or in the presence of the reagents employed. Such sensitivity/reactivity can interfere with the progress of the desired reaction to reduce the yield of the desired product, or possibly even preclude its formation. Accordingly, it may be necessary or desirable to protect sensitive or reactive groups on any of the molecules concerned. Protection can be achieved by means of conventional protecting groups, such as those described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 1991. The protecting groups may be removed at a convenient subsequent stage using methods known in the art. Alternatively the interfering group can be introduced into the molecule subsequent to the reaction step of concern. For example, if one or more of the substituents R¹, R², R³, and R⁴ in compound 1-2 can interfere with the coupling reaction between compounds 1-1 and 1-2 of Scheme 1, the substituent can be incorporated into the molecule in a post-coupling step to afford 1-3.

Scheme 5 exemplifies procedures which may be used for post-coupling 10 incorporation of suitable substituents into the azanapthalene core to obtain compounds of the invention, wherein coupled product 5-1 or 5-2 can be halogenated and the halogenated product 5-3 can be treated with a suitable nucleophile to provide Nu-substituted 5-4.

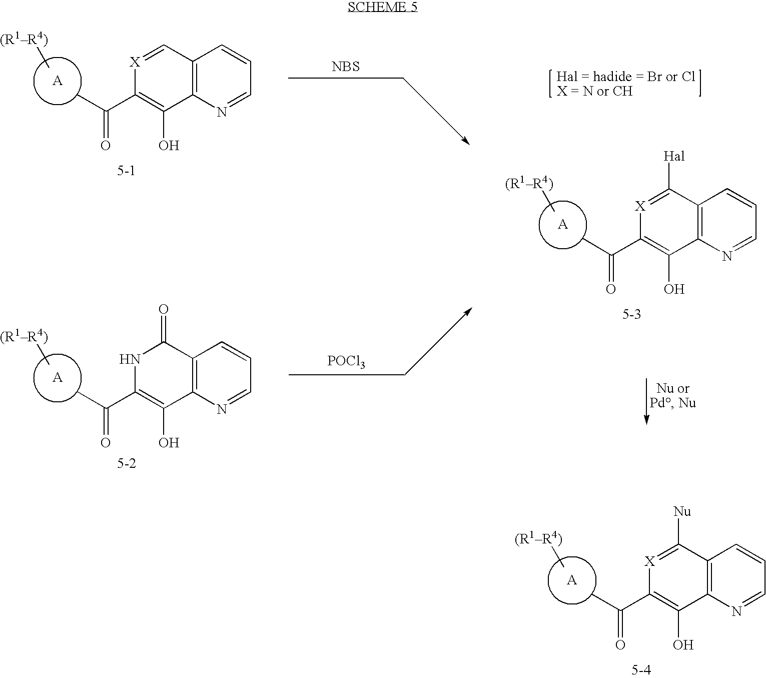

The following examples serve only to illustrate the invention and its practice. The examples are not to be construed as limitations on the scope or spirit of the invention.

### EXAMPLE 1

### 1-(3 -Benzylphenyl)- 1 -(8-hydroxyquinolin-7-yl)methanone

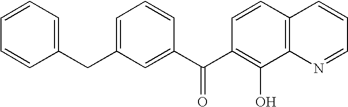 Step 1. 7-Bromoquinolin-8-ol (1a)

To a flame dried 100 mL 3 neck round bottom flask containing a stirring bar and fitted with a nitrogen inlet, addition funnel and a septum was added t-butylamine (7.24 mL, 68.89 mmol) in 50 mL toluene and the reaction was cooled to −78° C. To this was slowly added bromine (1.69 mL, 32.72 mmol) via syringe. The mixture was allowed to stir for 10 min, followed by the dropwise addition of 8-hydroxyquinoline (5 g, 34.45 mmol) in 10 mL chloroform via the addition funnel. The mixture was allowed to stir for 1 hr, then warmed to ambient temperature. The mixture was then diluted to 200 mL with ethyl acetate and extracted with saturated aqueous NaHCO₂, water, and brine. The organic extracts were dried over Na₂SO₄, filtered and the solvent removed to give the crude title material which was used in the next step without further purification.

ES MS M+1=224

Step 2. 7-Bromo-8-(2-methoxy-ethoxymethoxy)-quinoline (1b)

To a well dried 200 mL round bottom flask equipped with a stirring bar, septum, and nitrogen inlet was added 7-bromoquinolin-8-ol (3.1 g, 13.84 mmol), diisipropylethylamine (7.23 mL, 41.51 mmol) and 100 mL methylene chloride. MEM chloride (1.90 mL, 16.60 mmol) was then added dropwise to this mixture, and the reaction was allowed to stir 18 hours., after which another 0.95 mL (8.3 mmol) of MEM chloride was added. This mixture was stirred an additional 1 hr, then 50 mL water was added and the organic solvent removed in vacuo. The aqueous residue was extracted with three portions of EtOAc, and the combined organic extracts were washed with water, brine, dried (Na₂SO₄), filtered and the solvent removed in vacuo to give an oil. Subsequent silica gel chromatography (6:1 hexane/EtOAc→100% EtOAc) yielded 7-bromo-8-(2-methoxy-ethoxymethoxy)-quinoline.

¹H NMR (CDCl₃) δ: 3.37(3H, s); 3.61(2H, t, j=4.7Hz); 4.18(2H, t, j=4.7Hz); 5.75(3H, s); 7.43(1H, dd, j=8.3,4 Hz); 7.46(1H, d, j=9 Hz); 7.68(1H, d, j=8.8 Hz); 8.14(1H, dd, j=1.5,8.3 Hz); 8.90(1H, dd, j=1.6,4.2Hz)

Step 3. (3-Benzylphenyl){8-[(2-methoxyethoxy)methoxy]quinolin-7-yl}methanone (1c)

To a well dried 25 mL round bottom flask fitted with a stirring bar, an addition funnel, a nitrogen inlet and a septum was placed 7-bromo-8-(2-methoxyethoxymethoxy)-quinoline (0.766 g, 2.45 mmol) and 10 mL THF. The flask was cooled to −78° C., and to it was added t-butyllithium (3.6 mL of a 1.5M solution in pentane, 5.4 mmol) dropwise via syringe. The reaction was allowed to stir for 15 min, then N-methyl-N-methoxy-(3-benzyl)benzenecarboxyamide (0.626 g, 2.45 mmol) in 5 mL THF was added dropwise via addition funnel while maintaining the temp below −74° C. This mixture was stirred for 5 min, then allowed to warm to ambient temperature. The reaction was quenched by the addition of saturated aqueous NH₄Cl solution and extracted with EtOAc. The combined organic extracts were washed with water, brine, dried over Na₂SO₄, filtered and the solvent removed in vacuo. Silica gel chromatography (4:1 hexane/EtOAc→100% EtOAc) yielded (3-benzylphenyl){8-[(2-methoxyethoxy)methoxy]quinolin-7-yl }methanone.

¹H NMR (CDCl₃) δ: 3.22(3H, s), 3.17-3.25(5H, m); 3.51-3.59(2H, m); 4.13(2H, s); 5.55(2H, s); 7.15-7.75(11H, m); 7.85(1H, s); 8.19(1H, dd, j=1.5,8.2Hz); 8.97(1H, dd, j=1.6,4.2Hz) ES MS M+1=428

Step 4. 1-(3-Benzylphenyl)-1-(8-hydroxyquinolin-7-yl)methanone (1d)

To a 10 mL round bottom flask fitted with a stirring bar, nitrogen inlet and an addition funnel was added (3-benzylphenyl){8-[(2-methoxyethoxy)methoxy]quinolin-7-yl}methanone (0.2 g, 0.468 mmol) and 3 mL MeOH. Trifluoroacetic acid (1.081 mL, 14 mmol) was added dropwise, and the reaction was allowed to stir for 3 days, after which time it was poured into 20 mL aqueous saturated NaHCO₃ and extracted with EtOAc. The combined organic extracts were washed with water, brine, dried over NaSO₄, filtered and the solvent removed. Purification by reverse phase HPLC yielded 1-(3-Benzylphenyl)-1-(8-hydroxyquinolin-7-yl)methanone.

¹H NMR (CDCl₃) δ: 4.07(1H, s); 7.18-7.34(6H, m); 7.43(2H, d, j=4.8 Hz); 7.54(1H, dd, j=4.1,12.5 Hz); 7.57-7.70(3H, m); 8.1 1(1H, dd, j=1.3,8.3 Hz); 8.98(1H, d, j=2.7 Hz) ES MS M+1=340.

### EXAMPLE 2

### 1-(3-Benzylphenyl)-1-(8-hydroxy-4-methylquinolin-7-yl)methanone

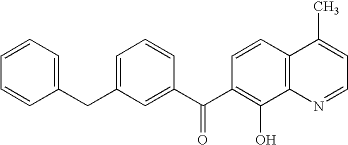 Step 1: 4-Methylquinolin-8-ol (2a)

Into a 100 mL round bottom flask containing a stirring bar and fitted with a reflux condenser and a septum was placed 10 mL 70% sulfuric acid, sodium iodide (0.23 g, 0.24 mmol) and anisidine (2.96 g, 24 mmol). This mixture was heated to 110° C., and to it was added methyl vinyl ketone (3.2 mL, 38.45 mmol) slowly over 5 hours via a syringe pump. After heating an additional hour, the reaction was cooled and poured into 50 mL 1M aqueous Na₂CO₃ and extracted with CH₂Cl₂. The combined organic extracts were extracted with 12M HCl. The acidic extracts were neutralized with 6M NaOH and extracted with CH₂Cl₂. The combined organic extracts were washed with water, brine dried over Na₂SO₄, filtered and the solvent removed. The residue was dissolved in EtOAc and passed through a silica pad to get the methyl ether, which was dissolved in 50 mL HBr and heated to reflux for 30 hours, after which the reaction was cooled and neutralized with 1ON NaOH and extracted with CH₂Cl₂. The combined organic extracts were combined, washed with water, brine, dried over Na₂SO₄, filtered and the solvent removed in vacuo to give 4-methylquinolin-8-ol.

¹H NMR (CDCl₃) δ: 2.70(3H, s); 7.18(1H, t, j=4Hz); 7.27(1H, d, j=4.2 Hz); 7.47(1H, d, j=4Hz); 8.63(1H, d,j=4.2 Hz) ES MS M+1=160

Step 2: 8—Hydroxy-4-methylquinoline-7-carboxylic acid (2b)

Into a 100 mL round bottom flask fitted with a stirring bar and a nitrogen inlet was placed 50 mL dry methanol. To this was added sodium (.163 g, 6.78 mmol) and the reaction was allowed to stir until all the metal was dissolved. 4-Methylquinolin-8-ol (0.83 g, 5.21 mmol) was added and the mixture stirred for 15 min, followed by removal of the solvent in vacuo. The resulting white solid was transferred to a high pressure reaction vessel, which was charged with CO₂ to 40 bar and heated to 170° C. for 3 days. After cooling and venting the gas, the brown residue was dissolved in water, filtered and acidified with 10% HCl. The water was removed in vacuo and thoroughly dried under hivac. The residue was slurried in MeOH, filtered and the solvent removed to give 8-hydroxy-4-methylquinoline-7-carboxylic acid.

¹H NMR (CDCl₃) δ: 3.02 (3H, s); 7.73(1H, d, j=8.9 Hz), 8.05(1H, d, j=5.1 Hz); 8.27(1 H, d, j=8.9 Hz); 9.28(1H, d, j=5.3 Hz) ES MS M+1=204

Step 3. Methyl 8-hydroxy-4-methylquinoline-7-carboxylate (2c)

8—Hydroxy-4-methylquinoline-7-carboxylic acid (0.4 g, 1.97 mmol) was dissolved in 25 mL MeOH and placed in a 50 mL round bottom flask fitted with a reflux condenser and a nitrogen inlet. Thionyl chloride (0.718 mL, 9.84 mmol) was carefully added, and the mixture was refluxed for 7 days, cooling and carefully adding an additional 0.718 mL of thionyl chloride daily for the first 5 days. The reaction was cooled and HCl gas carefully bubbled through it until saturated, then heated to reflux again for the last 2 days. Finally, the reaction was cooled, the solvent removed in vacuo, and the resulting residue partitioned between EtOAc and NaHCO₃ saturated water. After extraction, the combined organics were washed with water, brine, dried over Na₂SO₄, filtered and the solvent removed to give methyl 8-hydroxy-4-methylquinoline-7-carboxylate.

¹H NMR (CDCl₃) δ: 2.69(3H, s); 4.03(3H, s); 7.35(1H, s); 7.42(1H, d, j=8.9 Hz); 7.89(1H, d, j=8.8 Hz); 8.83(1H, s) ES MS M+1=218

Step 4. Methyl 8-[(2-methoxyethoxy)methoxy]-4-methylquinoline-7-carboxylate (2d)

Into a 15 mL round bottom flask fitted with a stirring bar, nitrogen inlet and septum was added methyl 8-hydroxy-4-methylquinoline-7-carboxylate (0.083 g, 0.38 mmol), N,N-diisopropylethylamine (1.99 mL, 1.15 mmol) and 5 mL CH₂Cl₂. To this was added MEM chloride (0.052 mL, 0.46 mmol) dropwise via syringe. After stirring for 1 hour, another equivalent of MEM chloride (0.052 mL, 0.46 mmol) was added. The reaction was stirred for an additional hour, after which time it was poured into water and the mixture was extracted with EtOAc. The combined organic extracts were washed with water, brine, dried over Na₂SO₄ filtered and the solvent removed in vacuo to provide methyl 8-[(2-methoxyethoxy)methoxy]-4-methylquinoline-7-carboxylate.

¹H NMR (CDCl₃) δ: 2.68(3H, s); 3.34(3H, s); 3.55(2H, t, j=4.6 Hz); 3.96(3H, s); 4.05(2H, t, j=4.6 Hz); 5.65(2H, s); 7.27(1H, d, j=4.2 Hz); 7.72(1H, d, j=8.9 Hz); 7.85(1H, d, j=8.8 Hz); 8.79(1H, d, j=4.2 Hz)

Step 5. 1-(3-Benzylphenyl)-1-(8-hydroxy-4-methylquinolin-7-yl)methanone (2e)

Into a flame dried 10 mL round bottom flask fitted with a stirring bar, nitrogen inlet and a septum was added 1-benzyl-3-bromobenzene (0.054 g, 0.22 mmol) and 2 mL THF. This mixture was cooled to −78° C. and to it was added t-butyllithium (0.29 mL of a 1.5 M solution in pentane, 0.43 mmol) slowly via syringe. The reaction was allowed to warn to 0° C., then cooled back down to −78° C. Into a separate 100 mL round bottom flask fitted with a stirring bar, nitrogen inlet and septum was added methyl 8-[(2-methoxyethoxy)methoxy]-4-methylquinoline-7-carboxylate (0.06 g, 0.197 mmol) and 50 mL THF. The contents of the first flask were transferred to the second via syringe, dropwise. After stirring for 1 hour, another ½ equivalent of 1-benzyl-3-bromobenzene (0.027 g, 0.11 mmol) and t-butyllithium (0.14 mL of a 1.5 M solution in pentane, 0.22 mmol) were reacted as above and added to the second flask. This mixture was stirred for an additional hour, then quenched by the addition of 10 mL aqueous saturated NH₄Cl solution and the THF removed in vacuo. The residue was partitioned between EtOAc and water, and extracted. The combined organic extracts were washed with water, brine, dried over Na₂SO₄, filtered and the solvent removed. The crude material was reverse phase chromatographed to get 0.022 g impure (3-benzylphenyl){8-[(2-methoxyethoxy)-methoxy]-4-methylquinolin-7-yl }methanone, then dissolved in 10 mL 95% MeOH, and 1 drop of conc. HCl added. This mixture was stirred 18 hours, after which another drop of conc. HCl was added and stirring continued for 3 hours. The solvent was removed and the residue triturated with ethyl ether to get a yellow solid. This material was placed on a Gilson Autoprep and the resulting product dissolved in EtOAc. HCl gas was bubbled briefly through this solution and the solvent removed in vacuo to afford 1-(3-benzylphenyl)-1-(8-hydroxy-4-methylquinolin-7-yl)methanone as the hydrochloride salt.

Free base ¹H NMR (CDCl₃) δ: 2.86(3H, s); 4.08(2H, s); 7.20-7.27(4H, m); 7.32(2H, t, j=7.5 Hz); 7.44-7.53(3H, m); 7.55-7.62(2H, m); 7.69(1H, s); 7.83(1H, d, j=8.9 Hz) ES MS M+1=354

### EXAMPLE 3

### 1-(3-benzylphenyl)-1-(8-hydroxy-5-methylquinolin-7-yl)methanone

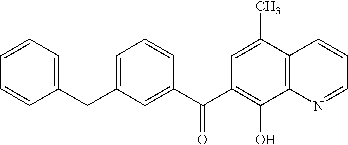 Step 1. N-methyl-N-methoxy-(3-benzoyl)benzenecarboxamide (3a).

To a 200 mL round bottomed flask with a stirring bar, reflux condenser and a drying tube was added 3-benzoylbenzoic acid (10.00 g, 44.20 mmol) and thionyl chloride (25 mL, 342.7 mmol). This mixture was heated at reflux for 3h. The thionyl chloride was removed in vacuo. The residue was dissolved in toluene and concentrated again to remove trace amounts of residual thionyl chloride. To a three necked, 1L round bottomed flask with a stirring bar, N₂ inlet and an addition funnel was added N, O-dimethylhydroxylamine hydrochloride (5.36 g, 55.00 mmol) and chloroform (160 mL). This solution was cooled in an ice bath and Et₃N (14.0 mL, 100 mmol) was added. The addition funnel was charged with a solution of the acid chloride in chloroform (40 mL) and this solution was added dropwise to the well stirred hydroxylamine solution over 30 min. The cooling bath was allowed to expire and the solution was stirred at ambient temperature, overnight. The reaction mixture was washed with dilute HCl, water and brine. Drying (MgSO₄), filtration and removal of the solvent in vacuo gave N-methyl-N-methoxy-(3-benzoyl)benzenecarboxamide as a foam. This material was used without further purification.

¹H NMR (CDCl₃) δ: 3.37(3H, s); 3.56(3H, s); 7.55(4H, m); 7.79(2H, d, j=6 Hz), 7.92(2H, d, j=6 Hz); 8.10(1H,s).

Step 2. N-methyl-N-methoxy-(3-benzyl)benzenecarboxamide (3b).

To a 500 mL Parr flask was added N-methyl-N-methoxy-(3-benzoyl)benzenecarboxamide (11.90 g, 44.2 mmol), absolute EtOH (100 mL), 10% Pd—C (1.00 g) and 70% HClO₄ (0.10 mL). The resulting mixture was hydrogenated on a Parr shaker at 70 psig fro 24 h. The catalyst was removed by filtration on a celite pad and the solvent was removed in vacuo. The crude product was chromatographed on 400 g of silica gel using 40% EtOAc/hexanes as eluant to give N-methyl-N-methoxy-(3-benzyl)benzenecarboxamide as an oil.

¹H NMR (CDCl₃) δ: 3.32(3H, s); 3.5o(3H, s); 4.01(2H,s); 7.19(3H, m); 7.28(4H, m), 7.51(2H, br s).

Step 3. 8-Methoxy-5-methylquinoline (3c).

To a 300 mL three necked round bottomed flask with a stirring bar, reflux condenser and a septum was added 2-methoxy-5-methylaniline (15.00 g, 109.3 mmol), sodium iodide (0.15 g, 1.00 mmol) and 70% aqueous sulfuric acid (20.6 mL, 260 mmol). This well stirred mixture was heated in an oil bath at 1 10° C. and acrolein (14.6 mL, 218 mmol) was added with a syringe pump over 3 h. When the addition was complete the reaction was maintained at 110° C. for an additional hour. The cooled mixture was diluted with water and partitioned between EtOAc and additional water. The layers were separated and the aqueous phase was filtered through a celite pad. This dark brown solution was basified with 50% NaOH (30 mL). The milky mixture was extracted with two portions of chloroform. The combined chloroform fractions were washed with brine, dried (MgSO₄), filtered and concentrated in vacuo to give 8-methoxy-5-methylquinoline.

¹H NMR (CDCl₃) δ: 2.60(3H,s); 4.07(3H,s); 6.94(1H, d, j=8 Hz); 7.28(1H, d, j=8 Hz); 7.46(1H, dd, j=4,8 Hz); 8.27(1H, dd, j=1.5,8Hz); 8.94(1H, dd, j=1.5,4 Hz).

Step 4. 8—Hydroxy-5-methylquinoline (3d).

To a 1L round bottomed flask with a stirring bar and a reflux condenser was added 8-methoxy-5-methylquinoline (17.04 g, 98.38 mmol) and 48% aqueous HBr (150 mL, 1.325 mol). This mixture was heated at reflux for 35h. The mixture was cooled to ambient temperature and the HBr was removed in vacuo. The residue was dissolved in water and basified with NH₄OH solution. The milky mixture was extracted with three portions of chloroform. The combined chloroform extracts were washed with brine, dried (MgSO₄), filtered and concentrated in vacuo to give 8-hydroxy-5-methylquinoline as off-white crystals.

¹H NMR (CDCl₃) δ: 2.59(3H,s); 7.07(1H, d, j=8Hz); 7.28(1H, d, j=8 Hz); 7.46(1H, dd, j=4,8 Hz); 8.16(1H, br s); 8.27(1H, dd, j=1.5,8 Hz); 8.79(1H, dd, j=1.5,4 Hz).

Step 5. 8—Hydroxy-7-bromo-5-methylquinoline (3e).

To a 1L round bottomed flask with a stirring bar, nitrogen inlet low temperature thermometer and a constant rate of addition funnel was added toluene (400 mL) and tert-butylamine (20.34 mL, 73.14 mmol). This solution was cooled to −78° C. and bromine (3.32 mL, 64.52 mmol) was added in one portion. The addition funnel was charged with a solution of 8-hydroxy-5-methylquinoline (10.27 g, 64.52 mmol) in chloroform (200 mL). This solution was added dropwise over 45 min. to the brominating reagent. The cooling bath was allowed to expire and the mixture warm to ambient temperature. The mixture was diluted with chloroform and and washed with 1L of water and brine. Drying (MgSO₄), filtration and removal of the solvent in vacuo gave 8-hydroxy-7-bromo-5-methylquinoline as a solid.

¹H NMR (CDCl₃) δ: 2.46(3H,s); 7.24(1H, m); 7.45(1H, s); 7.82(1H, d, j=4 Hz); 8.21(1H, d, j=8 Hz); 8.32(1H, br s).

Step 6. 8-(2-Methoxyethoxy)methoxy-7-bromo-5-methylquinoline (3f).

To a 500 mL round bottomed flask with a stirring bar and a nitrogen inlet was added 8-hydroxy-7-bromo-5-methylquinoline (8.92 g, 37.35 mmol), chloroform (250 mL) and N,N-diisopropylethylamine (39.03 mL, 224.1 mmol). This solution was cooled in an ice bath to 0° C. and MEM chloride (8.53 mL, 74.70 mmol) was added in one portion. The ice bath was allowed to expire and the mixture was stirred at ambient temperature 24h. The solution was recooled to 0° C. and another equivalent of MEM chloride (4.27 mL, 37.35 mmol) was added. The mixture was allowed to warm to ambient temperature and stirred another 24 h. This solution was washed with 10% aqueous citric acid, saturated NaHCO₃ solution and brine. Drying (MgSO₄), filtration and removal of the solvent in vacuo gave an oil. This material was chromatographed on 250 g of silica gel using 1:1 EtOAc:hexane as eluant. The purified product was triturated with hexane and the solid was collected by filtration and dried in vacuo to give 8-(2-methoxyethoxy)methoxy-7-bromo-5-methylquinoline as a white solid.

¹H NMR (CDCl₃) δ: 2.61(3H,s); 3.39(3H,s); 3.61(2H,m); 4.16(2H,m); 5.69(2H,s); 7.42(1H, dd, j=4,8 Hz); 7.52(1H, s); 8.26(1H, dd, j=1.5,8 Hz); 8.89(1H, dd, j=1.5,4 Hz).

Step 7. 1-(3-Benzylphenyl)-1-(8-(2-methoxyethoxy)methoxy-5-methylquinolin-7-yl)methanone (3g).

To an oven dried, three necked, 100 mL round bottomed flask with a stirring bar, nitrogen inlet and a low temperature thermometer was added 8-(2-methoxyethoxy)methoxy-7-bromo-5-methylquinoline (0.50 g, 1.53 mmol) and fresly distilled THF (15 mL). This solution was cooled to −78° C. and a solution of n-butyllithium (0.612 mL of a 2.5M solution in hexane, 1.53 mmol) was added dropwise with a syringe over 5 min. The resulting deep yellow solution was aged 5 min. then a solution of N-methyl-N-methoxy-(3-benzyl)benzenecarboxamide (0.39 g, 1.53 mmol) in 5 mL of THF was added over 3 min. with a syringe. The cooling bath was removed and the solution was warmed to ambient temperature. He reaction mixture was poured into saturated aqueous NH₄Cl solution and extracted with two portions of EtOAc. The combined EtOAc extracts were washed with brine, dried (MgSO₄), filtered and concentrated in vacuo. The crude product was chromatographed on 30 g of silica gel using 1:1 EtOAc:hexanes as eluant to give 1-(3-benzylphenyl)-1-(8-(2-methoxyethoxy)methoxy-5-methylquinolin-7-yl)methanone as a foam.

¹H NMR (CDCl₃) δ: 2.64(3H,s); 3.25(5H,m); 3.51(2H,m); 4.02(2H,m); 5.44(2H,s); 7.19(3H,m); 7.32(5H,m); 7.48(1H, dd, j=4,8Hz); 7.68(1H, d, j=8 Hz); 7.84(1H,s); 8.32(1H, dd, j=1.5,8 Hz); 8.96(1H, dd, j=1.5,4 Hz).

Step 8. 1-(3-Benzylphenyl)-1-(8-hydroxy-5-methylquinolin-7-yl)methanone (3h).

To a 50 mL round bottomed flask with a stirring bar was added 1-(3-benzylphenyl)-1-(8-(2-methoxyethoxy)methoxy-5-methylquinolin-7-yl)methanone (0.298 g, 0.67 mmol), methanol (10 mL) and trifluoroacetic acid (5 mL). The flask was stoppered and the solution was stirred at ambient temperature 90h. The solvents were removed in vacuo. The residue was dissolved in EtOAc (100 mL) and this solution was washed with saturated aqueous NaHCO₃ solution and brine. Drying (MgSO₄), filtration and removal of the solvent in vacuo gave a yellow, crystalline solid. This material was triturated with a little ethyl ether and the crystals were collected on frit then dried in vacuo to give 1-(3-benzylphenyl)-1-(8-hydroxy-5-methylquinolin-7-yl)methanone. Mp: 123-124° C.

¹H NMR (CDCl₃) δ: 2.51(3H,s); 4.07(2H,s); 7.22(6H,m); 7.43(3H,m); 7.61(3H, m); 8.28(1H, dd, j=1.5,8 Hz); 8.99(1H, dd, j=1.5,4 Hz). ES MS M+1=354

### EXAMPLE 4

### [3-Benzyl-5-(1H-1,2,4-triazol-1-ylmethyl)phenyl](5-chloro-8-hydroxyquinolin-7-yl)methanone

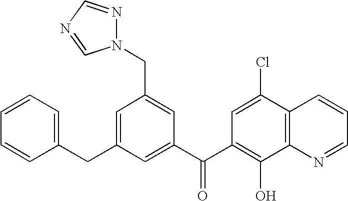 Step 1. N,N′-dimethoxy-N,N′,5-trimethylisophthalamide (4a).

To a 200 mL round bottomed flask with a stirring bar, reflux condenser and a drying tube was added 5-methylisophthalic acid (10.00 g, 55.51 mmol) and thionyl chloride (50.0 mL, 685.47 mmol). This mixture was heated at 50° C. 18h. The excess thionyl chloride was removed in vacuo to give solid crude diacid chloride. To a 1L, three necked round bottomed flask with a stirring bar, nitrogen inlet and an addition funnel was added N,O-dimethylhydroxylamine hydrochloride (14.63 g, 150 mmol) and chloroform (250 mL). This solution was cooled to 0° C. and triethylamine (42 mL, 300 mmol) was added. The addition funnel was charged with a solution of diacid chloride in chloroform (50 mL). This solution was added to the reaction mixture dropwise over 1 h. The cooling bath was allowed to expire and the mixture was stirred at ambient temperature 18 h. The reaction mixture was transferred to a separatory funnel and washed sequentially with water, 1N HCl, water and brine. Drying (MgSO₄), filtration and removal of the solvent in vacuo gave an oil. The product was crystallized from 10% EtOAc:hexane, collected on a frit and dried to give N,N′-dimethoxy-N,N′,5-trimethylisophthalamide as white crystals.

¹H NMR (CDCl₃) δ: 2.42(3H,s); 3.35(6H,s); 3.55(6H,s); 7.58(2H,s); 7.77(1H,s).

Step 2. 3-Benzoyl-N-methoxy-N,5-dimethylbenzamide (4b).

To a 500 mL, three necked round bottomed flask with a stirring bar, nitrogen inlet and a low temperature thermometer was added N,N′-dimethoxy-N,N′,5-trimethylisophthalamide (12.70 g, 47.69 mmol) and 300 mL of dry THF. This solution was cooled to −60° C. and a solution of phenylmagnesium bromide (16.21 mL of a 3.0 M solution in ethyl ether, 48.65 mmol) was added with a syringe. The solution was warmed to ambient temperature and stirred for 24 h. The reaction was quenched by addition of saturated aqueous NH₄Cl solution. The mixture was extracted with EtOAc. The organic fraction was washed with water and brine. Drying (MgSO₄), filtration and removal of the solvent in vacuo gave an oil. This material was chromatographed on 250 g of silica gel using 1:1 EtOAc:hexane as eluant to give 3-benzoyl-N-methoxy-N,5-dimethylbenzamide as an oil.

¹H NMR (CDCl₃) δ: 2.46(3H,s); 3.35(3H,s); 3.55(3H,s); 7.49(2H,m); 7.58(1H,m); 7.78(2H,m); 7.81(3H,m).

Step 3. 3-Benzoyl-5-(bromomethyl)-N-methoxy-N-methylbenzamide (4c).

To a 500 mL round bottomed flask with a stirring bar, reflux condenser and a nitrogen inlet was added 3-benzoyl-N-methoxy-N,5-dimethylbenzamide (10.02 g, 35.37 mmol), N-bromosuccinimide (6.29 g, 35.37 mmol), a catalytic amount of azobisisobutyronitrile, and carbon tetrachloride (220 mL). This well stirred mixture was heated at reflux for 3 h. The cooled mixture was diluted with chloroform and washed with water, NaHCO₃ solution and brine. Drying (MgSO₄), filtration and removal of the solvent in vacuo gave an oil. This material was chromatographed on 250 g of silica gel using 45:55 EtOAc:hexane as eluant. The chromatographed product was triturated with 1:1 ethyl ether :hexane and collected on a frit to give 3-benzoyl-5-(bromomethyl)-N-methoxy-N-methylbenzamide as a white crystalline solid.

¹H NMR (CDCl₃) δ: 3.35(3H,s); 3.58(3H,s); 4.55(2H,s); 7.52(2H,m); 7.62(1H,m); 7.80(2H,m); 7.81(2H,m); 8.01(1H,br s).

Step 4. 3-Benzoyl-N-methoxy-N-methyl-5-(1H-1,2,4-triazol-1-ylmethyl)benzamide (4d).

To a 100 mL round bottomed flask with a stirring bar and a nitrogen inlet was added 3-benzoyl-5-(bromomethyl)-N-methoxy-N-methylbenzamide (1.00 g, 2.76 mmol), 1,2,4-triazole (0.57 g, 8.28 mmol), finely powdered K₂CO₃ (1.38g, 10.0 mmol) and dry acetonitrile (25 mL). This mixture was stirred at ambient temperature 72 h. The solids were removed by filtration and the filtrate was concentrated in vacuo. The residue was dissolved in EtOAc and washed with water and brine. Drying (MgSO₄), filtration and removal of the solvent in vacuo gave an oil. This material was chromatographed on 70 g of silica gel using 5:95 2-propanol:chloroform as eluant to give 3-benzoyl-N-methoxy-N-methyl-5-(1H-1,2,4-triazol-1-ylmethyl)benzamide as a foam.

¹H NMR (CDCl₃) δ: 3.35(3H,s); 3.51(3H,s); 5.46(2H,s); 7.51(2H,m); 7.63(1H,m); 7.80(3H,m); 7.81(1H,s); 8.05(1H,br s); 8.18(1H,s).

Step 5. 3-Benzyl-N-methoxy-N-methyl-5-(1H-1,2,4-triazol-1-ylmethyl)benzamide (4e).

To a 25 mL round bottomed flask with a stirring bar and a stopper was added 3-benzoyl-N-methoxy-N-methyl-5-(1H-1,2,4-triazol-1-ylmethyl)benzamide (0.225 g, 0.64 mmol) and anhydrous trifluoroacetic acid (5.0 mL). To this well stirred solution was added triethylsilane (2.00 mL, 12.59 mmol). The resulting biphasic mixture was stirred vigorously at ambient temperature for 24 h. The mixture was concentrated in vacuo and the residue was dissolved in EtOAc. The EtOAc solution was washed with NaHCO₃ solution and brine. Drying (MgSO₄), filtration and removal of the solvent gave a foam. This material was chromatographed on silica gel using 6:94 2-propanol:chloroform as eluant to give 3-benzyl-N-methoxy-N-methyl-5-(1H-1,2,4-triazol-1-ylmethyl)benzamide as a foam.

¹H NMR (CDCl₃) δ: 3.31(3H,s); 3.45(3H,s); 4.01 (2H,s); 5.35(2H,s); 7.22(6H,m); 7.43(1H,s); 7.51(1H,s); 8.05(1H,s); 8.25(1H,s).

Step 6. 3-Benzyl-5-(1H-1,2,4-triazol-1-ylmethyl)benzaldehyde (4f).

To a 100 mL round bottomed flask with a stirring bar and a nitrogen inlet was added 3-benzyl-N-methoxy-N-methyl-5-(1H-1,2,4-triazol-1-ylmethyl)benzamide (1.30 g, 3.86 mmol) and dry THF (20 mL). This solution was cooled to 0° C. and a solution of lithium aluminum hydride (3.86 mL, 3.86 mmol of a 1.0 M solution in THF) was added with a syringe. The resulting solution was stirred at 0° C. for 1h. The reaction was quenched with 25 mL of saturated aqueous sodium potassium tartrate solution, then stirred overnight at ambient temperature. The mixture was diluted with EtOAc and the layers were separated. The organic phase was washed with water and brine. Drying (MgSO₄), filtration and removal of the solvent in vacuo gave an oil. This material was chromatographed on 60 g of silica gel using 5:95 2-propanol:chloroform as eluant to give 3-benzyl-5-(1H-1,2,4-triazol-1-ylmethyl)benzaldehyde as a colorless oil.

¹H NMR (CDCl₃) δ: 4.04 (2H,s); 5.37(2H,s); 7.25(6H,m); 7.59(1H,s); 7.67(1H,m); 7.98(1H,s); 8.11(1H,s); 9.94(1H,s).

Step 7. 8—Hydroxy-7-iodo-5-chloroquinoline (4 g).

To a 500 mL round bottomed flask with a stirring bar, nitrogen inlet low temperature thermometer and a constant rate of addition funnel was added toluene (40 mL) and tert-butylamine (1.87 mL, 17.82 mmol). This solution was cooled to −78° C. and iodine (2.28 g, 9.00 mmol) in chloroform (40 mL) was added dropwise. The addition funnel was charged with a solution of 8-hydroxy-5-chloroquinoline (1.60 g, 8.91 mmol) in chloroform (20 mL). This solution was added dropwise over 45 min. to the iodinating reagent. The cooling bath was allowed to expire and the mixture warm to ambient temperature. The mixture was diluted with chloroform and and washed with water and brine. Drying (MgSO₄), filtration and removal of the solvent in vacuo gave 8-hydroxy-7-iodo-5-quinolinequinoline as a solid.

¹H NMR (CDCl₃) δ: 7.58(1H, dd, j=4,8 Hz); 7.88(1H, s); 8.49(1H, dd, j=1.5,8 Hz); 8.32(1H, dd, j=1.5,4 Hz).

Step 8. 8-(2-Methoxyethoxy)methoxy-7-iodo-5-chloroquinoline (4 h).

To a 200 mL round bottomed flask with a stirring bar and a nitrogen inlet was added 8-hydroxy-7-iodo-5-chloroquinoline (2.02 g, 6.61 mmol), chloroform (100 mL) and N,N-diisopropylethylamine (1.74 mL, 9.92 mmol). This solution was cooled in an ice bath to 0° C. and MEM chloride (1.13 mL, 9.92 mmol) was added in one portion. The ice bath was allowed to expire and the mixture was stirred at ambient temperature 24h. This solution was washed with 10% aqueous citric acid, saturated NaHCO₃ solution and brine. Drying (MgSO₄), filtration and removal of the solvent in vacuo gave an oil. This material was chromatographed on 80 g of silica gel using 3:7 EtOAc:hexane as eluant to give 8-(2-methoxyethoxy)methoxy-7-iodo-5-chloroquinoline as colorless needles.

¹H NMR (CDCl₃) δ: 3.37(3H,s); 3.61(2H,m); 4.16(2H,m); 5.74(2H,s); 7.52(1H, dd, j=4,8Hz); 7.97(1H, s); 8.52(1H, dd, j=1.5,8 Hz); 8.91(1H, dd, j=1.5,4 Hz).

Step 9. (+/−) [3-Benzyl-5-(1H-1,2,4-triazol-1-ylmethyl)phenyl]{5-chloro-8-[(2-methoxyethoxy)methoxy]-quinolin-7-yl}methanol (41).

To a 100 mL, three necked, oven dried, round bottomed flask with a stirring bar, nitrogen inlet, low temperature thermometer and a septum was added of 8-(2-methoxyethoxy)methoxy-7-iodo-5-chloroquinoline (0.551 g, 1.40 mmol) and dry THF (20 mL). This solution was cooled to −100° C. and tert-butyllithium (1.87 mL of a 1.5M solution in pentane, 2.80 mmol) was added with a syringe, keeping the temperature below −90° C. The resulting solution was aged 30 min. at −100° C. then a solution of 3-benzyl-5-(1H-1,2,4-triazol-1-ylmethyl)benzaldehyde (0.36 g, 1.30 mmol) in THF (5 mL) was added over 3 min.. The mixture was warmed to 0° C. and poured into saturated aqueous NaHCO₃ solution. This mixture was extracted with EtOAc. The organic fraction was washed with water and brine, dried (MgSO₄), filtered and concentrated in vacuo. The crude product was chromatographed on 50 g of silica gel using 5:95 2-propanol:chloroform as eluant to give (+/−) [3-benzyl-5-(1H-1,2,4-triazol-1-ylmethyl)phenyl]{5-chloro-8-[(2-methoxyethoxy)methoxy]-quinolin-7-yl}methanol.

¹H NMR (CDCl₃) δ: 13.34(3H,s); 3.57(2H,m); 3.71(1H,m); 3.92(2H,s); 4.13(1H,s); 4.57(1H, d, j=4 Hz); 5.25(2H,s); 5.53(1H,d, j=5 Hz); 5.75(1H, d, j=5 Hz); 6.62(1H,d, j=4 Hz); 6.92(1H,s); 7.21(7H, br m); 7.52(1H, dd, j=4,12 Hz); 7.95(1H,d, j=12 Hz); 8.52(1H, dd, j=1.5,8 Hz); 8.95(1H, dd, j=1.5,4 Hz).

Step 10. [3-Benzyl-5-(1H-1,2,4-triazol-1-ylmethyl)phenyl]{5-chloro-8-[(2-methoxyethoxy)methoxy]-quinolin-7-yl}methanone (4j).

To a 100 mL round bottomed flask with a stirring bar and a stopper was added (+/−) [3-benzyl-5-(1H-1,2,4-triazol-1-ylmethyl)phenyl]{5-chloro-8-[(2-methoxyethoxy)methoxy)-quinolin-7-yl}methanol (0.096 g, 0.18 mmol) chloroform (10 mL) and activated MnO₂ (0.40 g, 4.60 mmol). This mixture was stirred at ambient temperature 24 h. The mixture was filtered through a celite pad and the filtrate was concentrated in vacuo to give [3-benzyl-5-(1H-1,2,4-triazol-1-ylmethy])phenyl]{5-chloro-8-[(2-methoxyethoxy)methoxy]-quinolin-7-yl}methanone as an oil.

¹H NMR (CDCl₃) δ: 3.23(5H,m); 3.44(2H,m): 3.99 (2H,s); 5.31(2H,s); 5.47(2H,s); 7.29(3H,m); 7.28(4H,m); 7.62(2H,m); 7.73(1H, br s); 7.93(1H,s); 8.05(1H,s); 8.63(1H, dd, j=1.5,8 Hz); 9.02(1H, dd, j=1.5,4).

Step 11. 1-[3-Benzyl-5-(1H-1,2,4-triazol-1-ylmethyl)phenyl]-1-(5-chloro-8-hydroxyquinolin-7-yl)methanone (4k).

To a 100 mL round bottomed flask with a stirring bar and a stopper was added of [3-benzyl-5-(1H-1,2,4-triazol-1-ylmethyl)phenyl]{5-chloro-8-[(2-methoxyethoxy)methoxy]-quinolin-7-yl}methanone (0.094 g, 0.17 mmol), methanol (5 mL) and trifluoroacetic acid (5 mL). This solution was stirred 20 h at ambient temperature. The solvents were removed in vacuo. The residue was treated with ammonia saturated chloroform and concentrated again. This material was chromatographed by reverse phase chromatography on C18 silica using 0.1% TFA/water and acetonitrile as the mobile phase to give [3-benzyl-5-(1H-1,2,4-triazol-1-ylmethyl)phenyl](5-chloro-8-hydroxyquinolin-7-yl)methanone as a solid after lyophyllization.

Exact Mass: Calculated for C₂₆H₁₉ClN₄O₂+H⁺: M/Z=455.1271; Found: M/Z=455.1269. ¹H NMR (CDCl₃) δ: 4.08 (2H,s); 5.42(2H,s); 7.24(3H,m); 7.31(4H,m); 7.53(1H, br s); 7.60(1H, br s); 7.66(1H,s); 7.77(1H, dd, j=4,9 Hz); 8.10(1H,s); 8.37(1H,s); 8.62(1H, dd, j=1.5,9 Hz); 9.09(1H, dd, j=1.5,4 Hz).

### EXAMPLE 5

### 1-(3-Benzyl-5-imidazol-1-ylmethylphenyl)-1-(5-chloro-8-hydroxyquinolin-7-yl)methanone (5a)

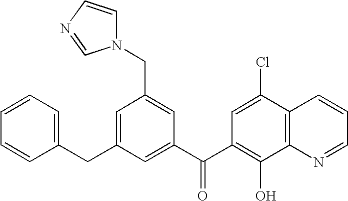

1-(3-Benzyl-5-imidazol-1-ylmethylphenyl)-1-(5-chloro-8-hydroxyquinolin-7-yl)methanone (5a) was prepared using a procedure similar to that described in Example 4.

MP: 148-150° C. (TFA salt). ¹H NMR (CDCl₃:DMSO-d₆ 1:1) δ: 4.04 (2H,s); 5.48(2H,s); 7.25(3H,m); 7.48(1H,s); 7.54(1H,s); 7.66(2H,m), 7.68(1H,s); 7.78(1H, dd, j=4,8 Hz); 7.94(1H,s); 8.56(1H,dd, j=1.2,8 Hz); 9.01(1H, dd, j=1.2,4 Hz); 9.21(1H,s). ES MS M+1=454

### EXAMPLE 6

### 1-(4-Benzyl-pyridin-2-yl)-1-(8-hydroxyquinolin-7-yl)methanone (6)

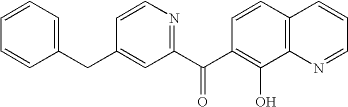 Step 1-4-Benzyl-pyridine N-oxide (6A)

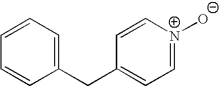

A mixture of 4-benzyl pyridine (15 mL, 94.0 mmol), acetic acid (90 mL) and hydrogen peroxide (35% aqueous solution, 30 mL) was heated at 85° C. overnight. After cooling to room temperature, the reaction mixture was treated with saturated aqueous sodium bicarbonate (300 mL) and extracted with dichloromethane three times. The combined organic phases were washed with brine, dried over magnesium sulfate and concentrated under vacuum to give 6A as a solid.
Step 2-4-Benzyl-pyridine-2-carbonitrile (6B)

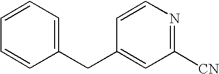

To a solution of 6A (14.0 g, 75.6 mmol) and triethylamine (16.0 mL) in acetonitrile (80 mL) was added trimethylsilyl cyanide (25.0 mL, 187.5 mmol) dropwise. The reaction mixture was then refluxed overnight. After cooling to room temperature, the reaction mixture was diluted with dichloromethane, washed with saturated aqueous sodium bicarbonate, brine and dried over NaSO₄, filtered and evaporated in vacuo. The residue was purified by flash chromatography (hexanes/ethyl acetate) to give 6B.
Step 3-4-Benzyl-pyridine-2-carboxylic acid methyl ester (6C)

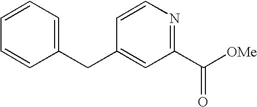

A solution of 6B (2.36 g, 12.1 mole) in methanol (50 ml) at 0° C. under argon was bubbled with hydrogen chloride gas till saturation. The reaction stirred at room temperature overnight. The solvent was removed under reduced pressure. The residue was treated with saturated aqueous NaHCO₃ and extracted with chloroform four times. The combined organic layers were washed with brine and dried over Na₂SO₄, filtered and evaporated. Chromatographic purification using ethyl acetate/hexanes afforded 6C.

Step 4-1-(4-Benzyl-pyridin-2-yl)-1-(8-hvdroxyquinolin-7-yl)methanone (6)

To a suspension of sodium hydride (60% in mineral oil, 106 mg, 2.65 mmol) in dry THF (15 mL) under argon was added 7-bromo-8-hydroxyquinoline (350 mg, 1.56 mmol) portionwise. After 1 h, the reaction mixture was cooled to −78° C. and n-butyl lithium (1.6 M in hexane, 1.07 mL, 1.71 mmol) was added. After 1 h, a solution of 6C (800 mg, 3.52 mmol) in THF (5 mL) was added. The reaction mixture warmed slowly to room temperature as the bath discharged overnight. The reaction was quenched with saturated aqueous ammonium chloride and extracted with chloroform three times. The combined organic layers were washed with brine, dried over Na₂SO₄, filtered and evaporated in vacuo. The residue was purified by preparative reverse phase HPLC to give 6.

¹H NMR (400 MHz, DMSO) δ 8.95 (d, 1H), 8.56 (d, 1H), 8.49 (d, 1H), 7.92 (s, 1H), 7.72-7.76 (m, 2H), 7.57 (d, 1H), 7.49 (d, 1H), 7.23-7.38 (m, 5H), 4.15 (s, 2H). ES MS M+1=341

### EXAMPLE 7

### 1-(3-Benzylphenyl)-1-(8-hydroxy-[1,6]naphthyridin-7-yl)methanone (7)

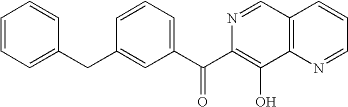 Step 1-(2—Chloro-pyridin-3-yl)methanol (7A)

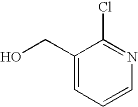

A mixture of 2-chloronicotinic acid(49.6 g) and thionyl chloride (250 mL) were heated to reflux under a drierite tube for 2 hours and aged at ambient temperature overnight. The mixture was concentrated under vacuum and the residue reconcentrated from toluene (2×) to remove residual thionyl chloride, to provide a tan solid. This crude acid chloride was then added in several portions to a solution of sodium borohydride (42 g) in deionized water (500 mL) which was maintained at 10-15 C. with an ice water bath during the addition. After the addition the mixture was stirred for 1 hour at ambient temperature, saturated with solid sodium chloride, and extracted with ether (3×300 mL). The combined extracts were dried over magnesium sulfate, filtered and concentrated under vacuum to provide 7A as a white solid.
Step 2-2—Chloro-3-chloromethylpyridine (7B)

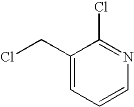

To a ice bath cooled mixture of 7A (15 g) in toluene (500 mL), under an atmosphere of nitrogen, was added over 5 minutes, thionyl chloride (11.5 mL). The cold bath was removed and the white slurry stirred at ambient temperature overnight. The mixture was then concentrated under vacuum and the residue partitioned between ether and saturated aqueous sodium bicarbonate. The aqueous layer was extracted with ether (3×) and the combined organic extracts washed with brine, dried over anhydrous magnesium sulfate, filtered and concentrated under vacuum to a pale yellow oil. Column chromatography on silica gel with 5-10 % ethyl acetate in hexane provided 7B as a colorless oil.
Step 3-3-Azidomethyl-2-chloropyridine (7C)

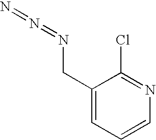

To a mixture of sodium azide (2.77 g) in anhydrous DMF (100 mL) under an atmosphere of nitrogen, was added 7B (6 g) in two portions. After stirring overnight, the reaction mixture was poured into a 1:1 mixture of 1M aqueous HCl, and brine (1L) and extracted with ether (3×). The combined extracts were dried over anhydrous sodium sulfate, filtered and concentrated under vacuum to provide the crude azide 7C as a colorless oil.
Step 4- (2-Chloropyridin-3-yl)methylamine (7D)

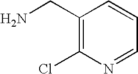

To a solution of crude 7C (9.8 g) in THF (250 mL) and deionized water (5 mL), was added triphenylphosphine (15.0 g) in portions over 5 minutes. After stirring overnight the reaction mixture was heated in a 35 C oil bath for 4 hours and aged at ambient temperature for 20 hours more. After concentrating under vacuum, the residue was subjected to column chromatography on silica gel eluting first with 10% methanol in ethyl acetate followed by 10% methanol in ethyl acetate containing 2% concentrated ammonium hydroxide. The product 7D isolated was a pale yellow oil.
Step 5-(3-Bromophenyl)phenylmethanol (7E)

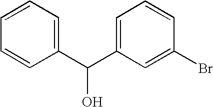

To an oven dried 500 ml 3-neck flask fitted with temperature probe, magnetic stir bar, and argon inlet was added a solution of 2.5M n-butyl lithium in hexanes (20.8 ml, 0.052 mole) chilled to −78° C. then diluted with diethyl ether (90 ml). To this was added dropwise by syringe over 30 minutes 1,3-dibromobenzene (11.80 g, 6.043 ml, 0.05 mole; activated basic alumina pretreatment) keeping the internal temperature between −74° C. and −78° C. The reaction was aged at −78° C. for 2.5h before adding neat benzaldehyde (5.52 g, 5.29 ml, 0.052 mole) over 15 minutes then allowing the reaction mixture to slowly warm to room temperature as the bath discharged overnight. The reaction was quenched with 20 mL H₂O then acidified with 5.4 ml conc. HCl and extracted with EtOAc three times. The combined organic layers were washed with NaHCO₃, brine and dried over NaSO₄, filtered and evaporated in vacuo to give a clear yellow oil 7E which crystallized to afford a white solid after washing with pet ether.
Step 6-(3-Benzyl)phenyl bromide (7F)

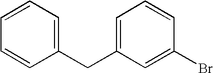

A solution of 7E (4.10 g, 0.0156 mole) and triethylsilane (2.72 g, 3.71 ml, 0.0234 mole) in methylene chloride (40 ml) was chilled to 0° C. under argon with stirring followed by addition of neat boron trifluoride etherate (3.32 g, 2.96 ml, 23.4 mmol). The reaction stirred at room temperature overnight. The reaction mixture was poured into 160 ml saturated NaHCO₃ and extracted with EtOAc three times, the combined organic layers were washed with brine and dried over Na₂SO₄, filtered and evaporated to afford colorless oil. Chromatographic purification using 5% EtOAc/hexanes afforded pure 7F.
Step 7-1-(3-Benzylphenyl)ethanone (7G)

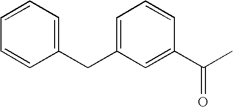

To an oven dried 100 ml 3-neck flask fitted with temperature probe, magnetic stir bar, and argon inlet was added 1.10 g 7F in 26 ml ThF and cooled to −78° C. Following dropwise addition of 1.6 M n-butyl lithium in hexanes (4.90 ml, 49 mmole) over 15 minutes, the reaction was stirred for 1 h at −78° C. before adding neat N-methoxy-N-methylacetamide (551 mg, 53.4 mmole) over 20 minutes. The reaction mixture warmed slowly to room temperature as the bath discharged overnight. The reaction was quenched with 60 ml 10% KHSO₄ and extracted with Et₂O three times. The combined organic layers were washed with NaHCO₃, brine and dried over Na₂SO₄, filtered and evaporated in vacuo to give a clear yellow oil. Chromatographic purification using EtOAc/hexanes afforded pure 7G.
Step 8-1-(3-Benzylphenyl)-2-bromo-ethanone (7H)

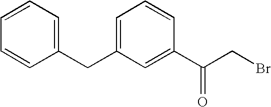

To a solution of 7G (3.0 g, 14.2 mmol) and aluminum chloride (190 mg, 1.4 mmol) in 1,4-dioxane (30 mL) was added bromine (0.77 mL, 15.0 mmol) at ambient temperature. After 20 min, the solvent was removed under reduced pressure. The residue was dissolved in ethyl acetate and washed with brine twice. The organic phase was dried over magnesium sulfate and concentrated to give product 7H as an oil.
Step 9-N-[2-(3-Benzylphenyl)-2-oxo-ethyl]—N-(2-chloro-pyridin-3-ylmethyl)-phenylsulfonamide (7I)

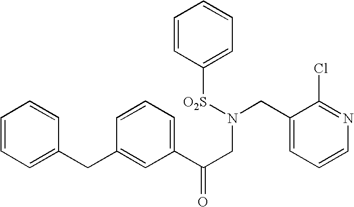

A solution of 2-chloropyridin-3-yl methylamine (7D)) (0.55 g) and diisopropylethylamine (2 mL) in anhydrous methylene chloride (10 mL) was cooled in an ice bath. To this was added a solution of 1-(3-benzylphenyl)-2-bromoethanone (7H) (1.12 g) in anhydrous methylene chloride (3 mL) over 5 minutes. The ice bath was removed and the mixture stirred 1 hr. After this time benzenesulfonyl chloride (0.54 mL) was added neat over 3 minutes and the mixture stirred at ambient temperature overnight. The reaction mixture was then concentrated under vacuum and the residue diluted with brine and extracted with methylene chloride (3×). The organic extracts were combined, dried over anhydrous sodium sulfate, filtered and concentrated under vacuum to a dark brown syrup. Column chromatography on silica gel with chloroform afforded the title compound as a pale yellow gum.
Step 10-3-({[2-(3-Benzylphenyl)-2-oxo-ethyl]-benzenesulfonylamino}-methyl)-pyridine-2-carboxylic acid ethyl ester (7J)

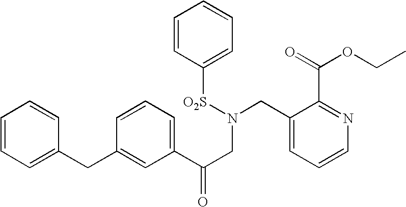

In a glass lined steel bomb was suspended N-[2-(3-benzylphenyl)-2-oxo-ethyl]—N-(2-chloro-pyridin-3-ylmethyl)-phenylsulfonamide (7I) (0.85 g) in absolute ethanol (20 mL). N-Methyl pyrrolidone (˜5 mL) was added to make a homogeneous solution which was bubbled with argon gas for 20 minutes. To this solution was then added sodium acetate (0.142 g), 1,3-bis(diphenylphosphino)propane (70 mg), and palladium acetate (38 mg). The vessel was sealed, purged with carbon monoxide (3×100 psi) then pressurized with carbon monoxide to 250 psi. The bomb was then heated in a 100° C. oil bath for 2 days. The pressure was relieved and the reaction mixture was filtered through Celite and the filtrate concentrated under vacuum to a reddish brown oil. Purification by column chromatography on silica gel with chloroform as the eluent provided the title compound as a colorless gum.

Step 11-1-(3-Benzylphenyl)-1-(8-hydroxy-[1,6]naphthyridin-7-yl)methanone (7)

To a solution of 3-({[2-(3-benzylphenyl)-2-oxo-ethyl]-benzenesulfonylamino}-methyl)-pyridine-2-carboxylic acid ethyl ester (7J) (0.85 g) in anhydrous THF(20 mL) was added solid sodium ethoxide (0.4 g) and the mixture stirred at ambient temperature under an atmosphere of nitrogen for 2 hours. The reaction was quenched with saturated aqueous ammonium chloride, diluted with deionized water and extracted with ethyl acetate (3×). The combined extracts were washed with brine, dried over anhydrous sodium sulfate, filtered and concentrated under vacuum to provide the crude title compound as a yellow foam. The material is subjected to reverse phase HPLC purification. Lyophilization of appropriate fractions provided the title compound.

¹H NMR (400 MHz, CD₃OD) δ 9.17 (dd, 1H), 8.90 (s, 1H), 8.61 (dd, 1H), 7.98-8.02 (m, 2H), 7.89 (dd, 1H), 7.42-7.52 (m, 2H), 7.16-7.30 (m, 5H), 4.08 (s, 2H). ES MS M+1=341

### EXAMPLE 8

### 1-[3-Benzyl-5-(1,1-dioxoisothiazolidin-2-ylmethyl)-phenyl]-1 -(8-hydroxy-[1,6]naphthyridin-7-yl)-methanone (8)

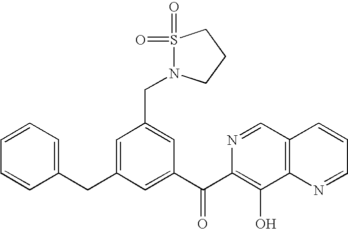 Step 1-(3-benzyl-5-bromo)benzyl bromide (8A)

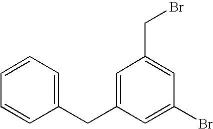

To a solution of alcohol (9d) (17 g, 61.4 mmol) and carbon tetrabromide (22.4 g, 67.5 mmol) in dichloromethane (200 mL) under argon was added triphenylphosphine (17.6 g, 67.5 mmol) in dichloromethane (20 mL) at 0° C. The reaction proceeded at ambient temperature overnight. The solvent was removed under reduced pressure. The residue was purified by flash chromotograph (hexanes/ethyl acetate) to give 8A as a solid.
Step 2-1-(1,1-dioxo-isothiazolidin-2-ylmethyl)-3-benzyl-5-bromo-benzene (8B)

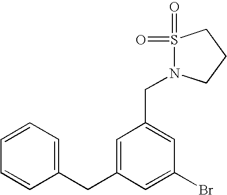

A mixture of 8A (5 g, 14.7 mmol), 1,3-propanesultam (3.6 g, 29.7 mmol) and potassium carbonate (4.1 g, 29.7 mmol) in acetonitrile (60 mL) was refluxed overnight. The solvent was removed under reduced pressure. The residue was partitioned between ethyl acetate and brine. The organic phase was dried over sodium sulfate, filtered and concentrated under reduced pressure. The residue was purified by flash chromatograph (ethyl acetate/hexanes) to give 8B as a white solid.
Step 3-{1-[3-Benzyl-5-(1,1-dioxo-isothiazolidin-2-ylmethyl)]-phenyl}ethanone (8C)

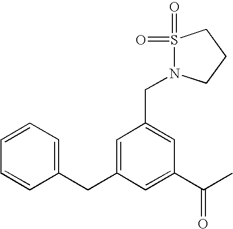

A sealed tube was charged with 8B (5 g, 13.2 mmol), thallium acetate (4.16 g, 15.8 mmol), 1,3-bis(diphenylphosphino)propane (0.98 g, 2.38 mmol), triethylamine (7.35 mL, 52.8 mmol) and DMF (20 mL). This mixture was purged with argon for 10 min. Palladium acetate (0.44 g, 1.98 mmol) and butyl vinyl ether (8.5 mL) were then added and the reaction mixture was stirred at 100° C. overnight. After cooling to room temperature, the reaction mixture was filtered through Celite. DMF was removed under vacuum. The residue was redissolved in THF (200 mL) and treated with 1N HCl (200 mL). After 1h, the reaction mixture was extracted with ethyl acetate twice. The combined organic layers were washed with brine, dried over sodium sulfate, filtered and concentrated. The residue was purified by flash chromatograph (hexanes/ethyl acetate) to give 8C as a white solid.
Step 4-1-{[3-Benzyl-5-(1,1-dioxo-isothiazolidin-2-ylmethyl)]-phenyl}-2-bromo-ethanone (8D)

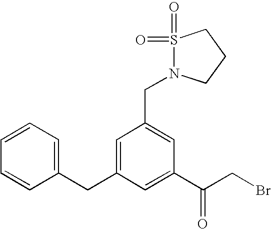

To a mixture of 8C (650 mg, 1.89 mmol) and aluminum chloride (7.6 mg, 0.05 mmol) in 1,4-dioxane (15 mL) was added bromine solution (0.62 M in 1,4-dioxane, 3.18 mL, 1.98 mmol) dropwise. After 1h, the solvent was removed in vacuo. The residue was purified by flash chromatograph (hexanes/ethyl acetate) to give 8D as an oil.
Step 5-{2-[3-Benzyl-5-(1,1-dioxo-isothiazolidin-2-ylmethyl)-phenyl]-2-oxo-ethyl}-(2-chloro-pyridin-3ylmethyl)-carbamic acid benzyl ester (8E)

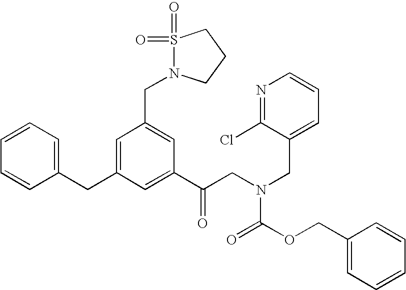

To a solution of amine 7D (292 mg, 1.74 mmol) and diisopropylethylamine (0.81 mL, 4.64 mmol) in acetonitrile (15 mL) was added 8D (490 mg, 1.16 mg) in acetonitrile (10 mL). After 1 h, benzyl chloroformate (0.5 mL, 3.48 mmol) was added. The reaction mixture was stirred overnight. The solvent was removed under reduced pressure. The residue was partitioned between ethyl acetate and brine. The organic layer was dried over sodium sulfate, filtered and concentrated. The residue was purified by flash chromatograph (hexanes/ethyl acetate) to give 8E as an oil.
Step 6-3-[({2-[3-Benzyl-5-(1,1 -dioxo-isothiazolidin-2-ylmethyl)-phenyl]-2-oxo-ethyl}-benzyloxycarbonyl-amino)-methyl]-pyridine-2-carboxylic acid ethyl ester (8F)

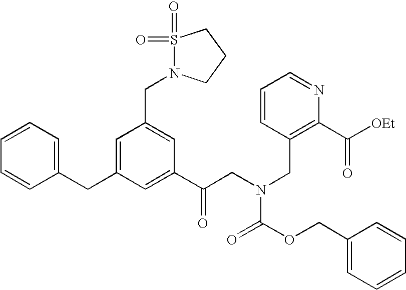

A mixture of 8E (500 mg, 0.81 mmol), trans-dichloro-bis(triphenylphosphine)palladium (II) (85.3 mg, 0.12 mmol) and triethylamine (1 mL) in ethanol (25 mL) and ethyl acetate (5 mL) in a par bomb flask was purged with argon for 10 min. Tha bomb was then pressurized with carbon monoxide to 250 psi and heated at 100° C. for 60 h. After cooling to room temperature, the reaction mixture was filtered through Celited and concentrated. The resultant residue was purified by flash chromatograph (ethyl acetate) to give 8F.

Step 7-1-[3-Benzyl-5-(1,1-dioxoisothiazolidin-2-ylmethyl)-phenyl]-1-(8-hydroxy-[1,6]naphthyridin-7-yl)-methanone (8)

To a solution of 8F (490 mg, 0.748 mmol) in dry THF (20 mL) was added sodium ethoxide (127 mg, 1.87 mmol) under argon. After 4h, water was added and the reaction mixture was extracted with ethyl acetate. The organic layer was dried over sodium sulfate, filtered and concentrated. The residue was then dissolved in acetonitrile (15 mL) and treated with 48% HBr (35 mL). This mixture was heated at 35° C. for 4h. The solvent and excess reagents were removed under vacuum. The resultant residue was purified by preparative reverse phase HPLC to give 8.

¹H NMR (400 MHz, DMSO) δ 12.0 (s, 1H), 9.20 (dd, 1H), 8.93 (s, 1H), 8.65 (dd, 1H), 7.88 (dd, 1H), 7.74 (s, 1H), 7.72 (s, 1H), 7.52 (s, 1H), 7.16-7.32 (m, 5H), 4.13 (s, 2H), 4.03 (s, 2H), 3.21 (t, 2H), 3.09 (t, 2H), 2.20 (quintet, 2H). ES MS M+1=474

### EXAMPLE 9

### 1-(3-Benzyl-5-(morpholin-4-ylmethyl)phenyl)-1-(8-hydroxy-[1,6]naphthyridin-7-yl)methanone

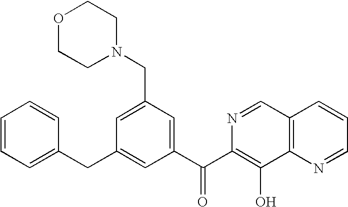 Step 1: (3,5-dibromophenyl)phenylmethanol (9a)

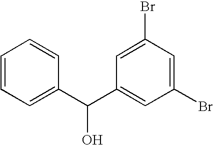

To a cold (−78° C.) solution of 1,3,5-tribromobenzene (30 g) in diethyl ether (500 mL), a solution of n-BuLi in hexanes (2.5 M, 38.1 mL) was added. The resultant mixture was stirred at −78° C. for 1 h and was treated with benzaldehyde (10.2 mL). The reaction mixture was allowed to warn up slowly to 0° C. and was stirred at that temp. for 1.5 hr. The product mixture was diluted with ethyl acetate and partitioned with aq. HCl (1M, 95 mL). The organic extract was washed with brine, dried over magnesium sulfate, filtered, and concentrated under vacuum to provide the title alcohol.
Step 2: 1-Benzyl-3,5-dibromobenzene (9b)

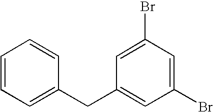

To a cold (0° C.) solution of (3,5-dibromophenyl)phenylmethanol (32.5 g) and triethylsilane (27.7 g) in dichloromethane (500 mL), boron trifluoride diethyl etherate (30 mL) was added dropwise over a period of 45 min. The resultant mixture was stirred at 0° C. for 1 hr, and at room temp. overnight. The product mixture was diluted with dichloromethane, and neutralized with saturated aq. sodium bicarbonate. The organic extract was washed with brine, dried over magnesium sulfate, filtered, and concentrated under vacuum. The residue was subjected to column chromatography on silica gel eluted with hexane. Collection and concentration of appropriate fractions provided the title dibromide.
Step 3. 3-Benzyl-5-bromobenzaldehyde (9c)

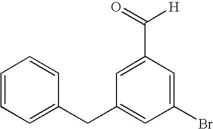

To a cold (−78 C.) solution of 1-benzyl-3,5-dibromobenzene (1.15 g) in THF (30 mL), a solution of n-BuLi in hexanes (2.5 M, 2 mL) was added. The resultant mixture was stirred at −78 C. for 1 h and was treated with anhydrous DMF (0.3 mL). The reaction mixture was allowed to warm up slowly to room temp. and was stirred at that temp. overnight. The product mixture was diluted with ethyl acetate and partitioned with aq. HCl. The organic extract was washed with brine, dried over magnesium sulfate, filtered, and concentrated under vacuum. The residue was subjected to column chromatography on silica gel eluting with 10% ethyl acetate in hexane. Collection and concentration of appropriate fractions provided the title benzaldehyde.
Step 4: 3-Benzyl-5-bromobenzyl alcohol (9d)

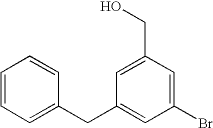

To a cold (0 C.) solution of 3-benzyl-5-bromobenzaldehyde (0.465 g) in methanol (5 mL), sodium borohydride (0.123 g) was added. The reaction mixture was stirred at room temp. for 3 hr. The product mixture was concentrated, and the residue partitioned between ethyl acetate and aq. HCl. The organic extract was washed with brine, dried over magnesium sulfate, filtered, and concentrated under vacuum to provide the title alcohol.
Step 5: 1-(3-Benzyl-5-hydroxymethyl-phenyl)-ethanone (9e)

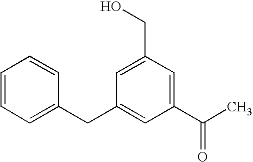

To a mixture of 3-Benzyl-5-bromobenzyl alcohol (10.8 g), thallium acetate (11.3 g), 1,3-bis(diphenylphosphino)propane (3.2 g) and triethylamine (16 mL) in DMF (60 niL) in a pressure tube, purged with argon for a period of 10 minute, palladium acetate (1.7 g) and n-butyl vinyl ether (25 mL) was added. The reaction tube was sealed and stirred at 100° C. overnight. The reaction mixture was filtered through a bed of Celite, and the filtrate concentrated under vacuum. The residue was dissolved in THF (200 mL) and treated with aq. HCl (1M, 100 mL). The resultant mixture was stirred at room temperature for 1 hr., diluted with methylene chloride and deionized water and the layers separated. The aqueous layer was extracted with methylene chloride (2×) and the combined organic extracts were dried over magnesium sulfate, filtered, and concentrated under vacuum. The residue was subjected to column chromatography on silica gel eluting with 25-35% ethyl acetate in hexane. Collection and concentration of appropriate fractions provided the title ketone.
Step 6: 1-(3-Benzyl-5-hydroxymethyl-phenyl)-2-bromo-ethanone (9f)

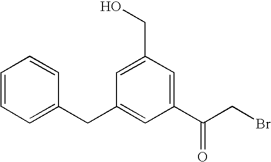

To a solution of 1-(3-benzyl-5-hydroxymethyl-phenyl)-ethanone (5.2 g) in anhydrous 1,4-dioxane (150 mL) was added a 100 mg/ mL solution of bromine in 1,4-dioxane (34.6 mL) over a 0.5 hr period. The mixture was stirred for 1 hr at ambient temp. and the solvent removed in vacuo. The residue was concentrated from toluene under vacuum (2X) to provide the crude product as an orange syrup and used immediately without further purification.
Step 7. [2-(3-Benzyl-5-hydroxymethyl-phenyl)-2-oxo-ethyl]-(2-chloro-pyridin-3-ylmethyl)-carbamic acid benzyl ester (9g)

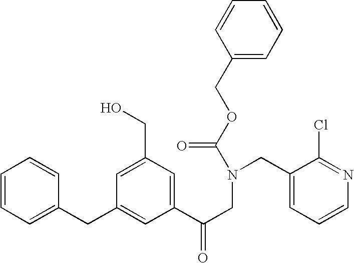

A solution of 2-chloropyridin-3-yl methylamine (3.1 g) and triethylamine (14.6 mL) in anhydrous DMF (100 mL) was cooled in an ice bath. To this was added a solution of 1-(3-Benzyl-5-hydroxymethyl-phenyl)-2-bromo-ethanone (7.5 g) in anhydrous DMF (100 mL) over 5 minutes. The ice bath was removed and the mixture stirred 1 hr. After this time benzyl chloroformate (3.3 mL) was added neat over 3 minutes and the mixture stirred at ambient temperature overnight. The reaction mixture was then concentrated under vacuum and the residue diluted with brine and extracted with methylene chloride (3×). The organic extracts were combined, dried over anhydrous sodium sulfate, filtered and concentrated under vacuum to a dark brown syrup. Column chromatography on silica gel with 50-60% ethyl acetate in hexane afforded the title compound as a pale yellow gum.
Step 8: 3-({[2-(3-Benzyl-5-hydroxymethyl-phenyl)-2-oxo-ethyl]-benzyloxycarbonyl-amino}-methyl)-pyridine-2-carboxylic acid ethyl ester (9h)

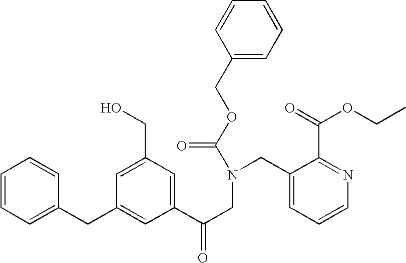

In a glass lined steel bomb was suspended [2-(3-benzyl-5-hydroxymethyl-phenyl)-2-oxo-ethyl]-(2-chloro-pyridin-3-ylmethyl)-carbamic acid benzyl ester (5.0 g) in absolute ethanol. N-Methyl pyrrolidone (˜9 mL) was added to make a homogeneous solution which was bubbled with argon gas for 20 minutes. To this solution was then added sodium acetate (0.88 g), 1,3-bis(diphenylphosphino)propane (0.2 g), and palladium acetate (0.1 g). The vessel was sealed, purged with carbon monoxide (3×100 psi) then pressurized with carbon monoxide to 250 psi. The bomb was then heated in a 100° C. oil bath for 3 days and aged 2 days at ambient temperature. The pressure was relieved and the reaction mixture was filtered through Celite and the filtrate concentrated under vacuum to a reddish brown oil. Purification by column chromatography on silica gel with 80% ethyl acetate in hexane to 100% ethyl acetate as the eluent provided the title compound as a colorless gum.
Step 9: 3-({[2-(3-Benzyl-5-bromomethyl-phenyl)-2-oxo-ethyl]-benzyloxycarbonyl-amino}-methyl)-pyridine-2-carboxylic acid ethyl ester (9i)

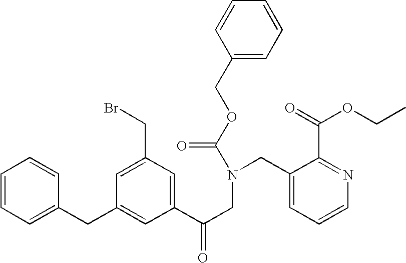

To a solution of 3-({ [2-(3-Benzyl-5-hydroxymethyl-phenyl)-2-oxo-ethyl]-benzyloxycarbonyl-amino 3-methyl)-pyridine-2-carboxylic acid ethyl ester (1.33 g) in anhydrous THF (25 mL) was added carbon tetrabromide (1.2 g), and triphenylphosphine (0.94 g). After stirring at ambient temperature for 1 hr. the reaction mixture was adsorbed onto silica gel and loaded onto a pre wetted silica gel column and eluted with 45% then 50% ethyl acetate in hexane. The appropriate fractions were combined and concentrated under vacuum to provide the title compound as a colorless gum.
Step 10: 3-({[2-(3-Benzyl-5-morpholin-4-ylmethyl-phenyl)-2-oxo-ethyl]-benzyloxycarbonyl-amino}-methyl)-pyridine-2-carboxylic acid ethyl ester (9j)

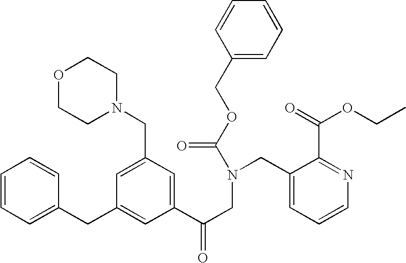

To a cold (0° C.) solution of 3-({[2-(3-benzyl-5-bromomethyl-phenyl)-2-oxo-ethyl]-benzyloxycarbonyl-amino}-methyl)-pyridine-2-carboxylic acid ethyl ester (0.41 g) in anhydrous DMF (7 mL) was added morpholine (0.17 mL) and potassium carbonate (0.11 g). The cooling bath was removed and the reaction mixture was stirred at ambient temperature overnight. The solvent was removed under vacuum and the residue partitioned between ethyl ether and brine. The layers were separated and the aqueous layer extracted with ethyl ether (2×). The combined ether extracts were backwashed with brine, dried over anhydrous sodium sulfate, filtered and concentrated under vacuum to a pink gum. Purification by column chromatography on silica gel and eluting with ethyl acetate followed by 5% THF in ethyl acetate provided the title compound as a pale red gum.
Step 11. 7-[1 -(3-Benzyl-5-morpholin-4-ylmethyl-phenyl)-methanoyl]-8-hydroxy-5H-[1,6]naphthyridine-6-carbamic acid benzyl ester (9k)

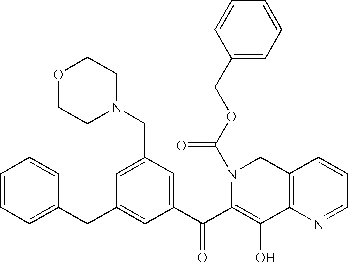

To a solution of 3-(( [2-(3-Benzyl-5-morpholin-4-ylmethyl-phenyl)-2-oxo-ethyl)-benzyloxycarbonyl-amino}-methyl)-pyridine-2-carboxylic acid ethyl ester (0.37 g,) in anhydrous THF (16 mL) was added solid sodium ethoxide (81 mg) and the mixture stirred at ambient temperature under an atmosphere of nitrogen. After 1 hour. additional sodium ethoxide (50 mg) was added and stirring continued. Another addition of sodium ethoxide (20 mg) after 2 hours. followed by an hour of stirring resulted in complete conversion. The reaction was quenched with saturated aqueous ammonium chloride, diluted with deionized water and extracted with ethyl acetate (3×). The combined extracts were washed with brine, dried over anhydrous sodium sulfate, filtered and concentrated under vacuum to provide the crude title compound as a yellow foam.

Step 12. 1-(3-Benzyl-5-morpholin-4-ylmethylphenyl)-1-(8-hydroxy-[1,6]naphthyridin-7-yl)methanone (9)

To a cold (0° C.) solution of 7-[1-(3-benzyl-5-morpholin-4-ylmethyl-phenyl)-methanoyl]-8-hydroxy-5H-[1,6]naphthyridine-6-carbamic acid benzyl ester (36 mg) in anhydrous acetonitrile (1 mL) under a nitrogen atmosphere was added trimethylsilyl iodide (22 μL). The mixture was stirred 0.5 hour at 0° C., and 1.5 hour at ambient temperature. An additional portion of trimethylsilyl iodide (22 μL) was added and stirring was continued for 1 hour. One hour after a third portion of trimethylsilyl iodide (22 μL) was added, the reaction mixture was concentrated under vacuum to a dark brown gum. The product was isolated by preparative HPLC with Waters PrepPak C18 cartridges and acetonitrile/water with trifluoroacetic acid modifier as the mobile phase, as a yellow orange lyophilized solid.

¹H NMR (400 MHz, CDCl₃) δ 9.3 (dd, 1H), 8.8 (s, 1H), 8.4 (dd, 1H), 8.2 (s, 2H), 7.8(dd, 1H), 7.5(s, 1H), 7.4-7.2 (m, 5H), 4.3 (s, 2H), 4.1 (s, 2H), 4.0 (m, 4H), 3.6 (m, 2H), 2.9 (m, 2H). ES MS M+1=440

### EXAMPLE 10

### 1-(3-Benzyl-5-piperidin-1 -ylmethyl-phenyl)-1-(8-hydroxy-[1,6]naphthyridin-7-yl)methanone

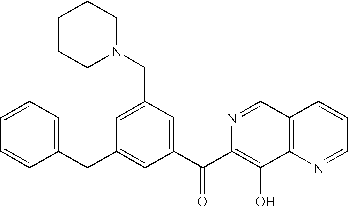

The title compound was prepared using a procedure similar to that described in Example 9, except that piperidine was substituted for morpholine in Step 10.

¹H NMR (400 MHz, CDCl₃) δ 11.0 (brs, 1H), 9.3 (d, 1H), 8.8 (s, 1H), 8.4 (d, 1H), 8.2 (s, 1H), 8.1 (s, 1H), 7.8 (dd, 1H), 7.6 (s, 1H), 7.4-7.2 (m, 5H), 4.3 (s, 2H), 4.1 (s, 2H), 3.6 (m, 2H), 2.7 (m, 2H), 2.1 (m, 2H), 2.1 (m, 2H).1.9 (m, 3H), 1.4 (m, 1H). ES MS M+1=438

### EXAMPLE 11

### 1-[3-Benzyl-5-(4-methylpiperazin-1-ylmethyl)phenyl]-1-(8-hydroxy-[1,6]naphthyridin-7-yl)methanone

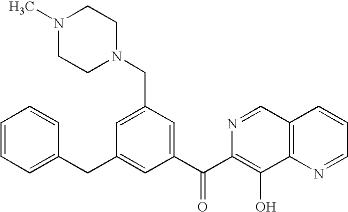

The title compound was prepared using a procedure similar to that described in Example 9, except that 4-methylpiperazine was substituted for morpholine in Step 10.

¹H NMR (400 MHz, CDCl₃) δ 9.3 (dd, 1H), 8.8 (s, 1H), 8.4 (dd, 1H), 8.2 (s, 1H), 8.1 (s, 1H), 7.8 (dd, 1H), 7.6 (s, 1H), 7.4-7.2 (m, 5H), 4.2 (s, 2H), 4.1 (s, 2H), 3.6 (m, 8H), 2.9 (s, 3H). ES MS M+1=453

### EXAMPLE 12

### 1-{3-Benzyl-5-[1-(8-hydroxy-[1,6]naphthyridin-7-yl)methanoyl]benzyl }-1H-pyridin-2-one

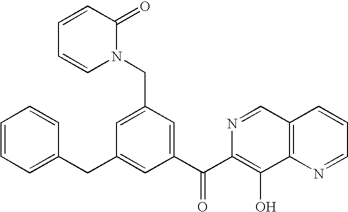

The title compound was prepared using a procedure similar to that described in Example 9, except that 2-hydroxypyridine was substituted for morpholine and cesium carbonate was substituted for potassium carbonate in Step 10.

¹H NMR (400 MHz, CDCl₃) δ 9.3 (dd, 1H), 8.8 (s, 1H), 8.4 (dd, 1H), 8.05 (s, 1H), 8.0 (s, 1H), 7.8 (dd, 1H), 7.5 (m, 1H), 7.4 (m, 2H) 7.3-7.2 (m, 5H), 6.9 (d,1H), 6.4 (dt, 1H), 5.3 (s, 2H), 4.1 (s, 2H). ES MS M+1=448

### EXAMPLE 13

### 3-{3-Benzyl-5-[(8-hydroxy-1,6-naphthyridin-7-yl)carbonyl]benzyl}-1-methylpyrimidine-2,4-(1H,3H)-dione

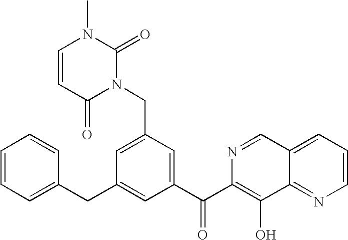

The title compound was prepared using a procedure similar to that described in Example 9, except that 1-methyluricil was substituted for morpholine and cesium carbonate was substituted for potassium carbonate in Step 10.

¹H NMR (400 MHz, CDCl₃) δ 9.3 (dd, 1H), 8.8 (s, 1H), 8.4 (dd, 1H), 8.2 (s, 1H), 7.9 (s, 1H), 7.8 (dd, 1H), 7.6 (s, 1H), 7.3-7.2 (m, 5H), 7.1 (d,1H), 5.8 (d, 1H), 5.2 (s, 2H), 4.1 (s, 2H), 3.4 (s, 3H). ES MS M+1=479

### EXAMPLES 14 AND 15

### 1-[3-Benzyl-5-(tetrazol-1-ylmethyl)phenyl]-1-(8-hydroxy-[1,6]naphthyridin-7-yl)methanone (14)

### 1-[3-Benzyl-5-(tetrazol-2-ylmethyl)phenyl]-1-(8-hydroxy-[1,6]naphthyridin-7-yl)methanone (15)

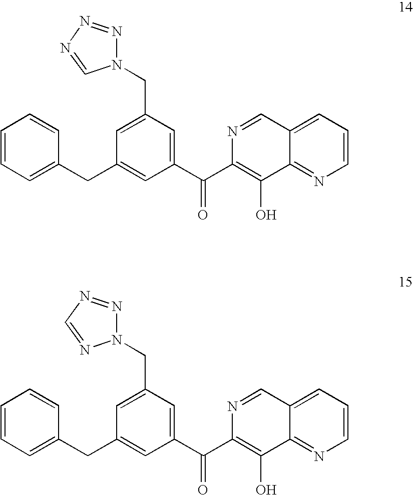

The title compounds were prepared using a procedure similar to that described in Example 9, except that tetrazole was substituted for morpholine and two regioisomers were isolated by column chromatography in Step 10. These were carried forward in a similar manner to provide the title compounds as yellow lyophilized solids.

14: ¹H NMR (400 MHz, CDCl₃) δ 9.3 (dd, 1H), 8.8 (s, 1H), 8.5 (s, 1H), 8.4 (dd, 1H), 8.1 (s, 1H), 8.0 (s, 1H), 7.8 (dd, 1H), 7.4-7.2 (m, 6H), 5.7 (s, 2H), 4.1 (s, 2H). ES MS M+1=423

15: ¹H NMR (400 MHz, CDCl₃) δ 9.4 (dd, 1H), 8.8 (s, 1H), 8.55 (s, 1H), 8.5 (dd, 1H), 8.1 (s, 1H), 8.05 (s, 1H), 7.9 (dd, 1H), 7.5 (s, 1H), 7.3-7.2 (m, 5H), 5.9 (s, 2H), 4.1 (s, 2H). ES MS M+1=423

### EXAMPLE 16

### 1-(3-Benzyl-5-pyrazol-1-ylmethylphenyl)-1-(8-hydroxy-[1,6]naphthyridin-7-yl)methanone

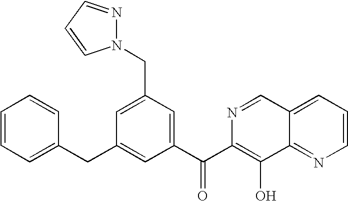 Step 1: 7-[1-(3-Benzyl-5-pyrazol-1-ylmethylphenyl)-methanoyl]-8-hydroxy-5H-[1,6]naphthyridine-6-carbamic acid benzyl ester

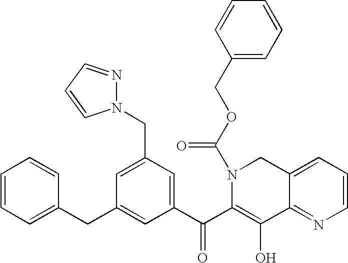

The title compound was prepared using a procedure similar to that described in Example 9, Steps 1 to 11, except that pyrazole was substituted for morpholine in Step 10.
Step 2: 1-(3-Benzyl-5-pyrazol-1-ylmethylphenyl)-1-(8-hydroxy-[1,6]naphthyridin-7-yl)methanone

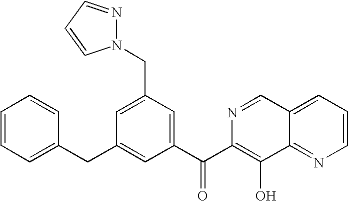

To a solution of 7-[1-(3-benzyl-5-pyrazol-1-ylmethylphenyl) methanoyl]-8-hydroxy-5H-[1,6]naphthyridine-6-carbamic acid benzyl ester (0.16 g) in acetonitrile (3 mL) was added 48% aqueous hydrobromic acid ( 5 mL). The mixture was heated in a 50 C oil bath for 5 hours and concentrated under vacuum to a dark red semisolid. This material was suspended in acetonitrile and treated with deionized water to dissolve all solids and heated in a 50 C oil bath while exposed to air for 8 hours. After cooling to ambient temperature the reaction was diluted with deionized water and extracted with chloroform (3×). The combined organic extracts were dried over anhydrous sodium sulfate, filtered and concentrated under vacuum to a reddish brown gum. The product was isolated by preparative HPLC with Waters PrepPak C18 cartridges and acetonitrile/ water with trifluoroacetic acid modifier as the mobile phase, as a yellow orange lyophilized solid.

¹H NMR (400 MHz, CDCl₃) δ 9.4 (dd, 1H), 8.8 (s, 1H), 8.5 (dd, 1H), 8.0 (s, 1H), 7.95 (s, 1H), 7.85 (dd, 1H), 7.7 (d, 1H), .7 5(d, 1H), 7.35-7.2 (m, 6H), 6.4 (t, 1H), 5.5 (s, 2H), 4.1 (s, 2H). ES MS M+1=421

### EXAMPLE 17

### 3-{3-Benzyl-5-[1-(8-hydroxy-[1,6]naphthyridin-7-yl)methanoyl]benzyl}-3H-pyrimidin-4-one

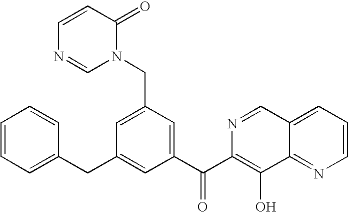

The title compound was prepared using a procedure similar to that described in Example 16, except that 4(3H) pyrimidone was substituted for pyrazole and cesium carbonate was substituted for potassium carbonate in Step 10.

¹H NMR (400 MHz, CDCl₃) δ 9.3 (dd, 1H), 8.8 (s, 1H), 8.5 (s, 1H), 8.4 (dd, 1H), 8.1 (s, 1H), 8.0 (s, 1H), 7.9 (d, 1H), 7.8 (dd, 1H), 7.5 (s, 1H), 7.35-7.2 (m, 5H), 6.6 (d,1H), 5.2 (s, 2H), 4.1 (s, 2H). ES MS M+1=449

### EXAMPLE 18

### 1- {3-Benzyl-5-[1 -(8-hydroxy-[1 ,6]naphthyridin-7-yl)methanoyl]benzyl}pyrrolidin-2-one

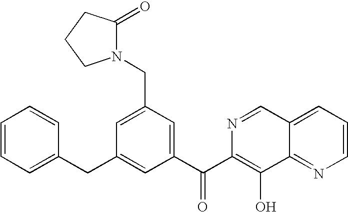 Step 1: 7-{1-[3-Benzyl-5-(2-oxo-pyrrolidin-1-ylmethyl)-phenyl]-methanoyl}-8-hydroxy-5H-[1,6]naphthyridine-6-carbamic acid benzyl ester.

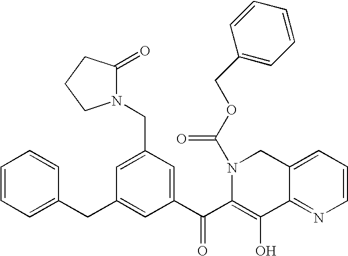

To a suspension of 60% oil dispersion of sodium hydride (48 mg) in anhydrous DMF (5 mL) was added pyrrolidinone (105 mg) in one portion. The mixture was stirred at ambient temperature for 1 hour and then treated with a solution of 3-({[2-(3-benzyl-5-bromomethyl-phenyl)-2-oxo-ethyl]-benzyloxycarbonylamino}-methyl)-pyridine-2-carboxylic acid ethyl ester (0.41 g) in anhydrous DMF (5 mL). After stirring overnight, the reaction mixture was treated with saturated aqueous ammonium chloride, diluted with deionized water and extracted with ethyl acetate (3×). The combined organic extracts were backwashed with deionized water, brine (2×), dried over anhydrous sodium sulfate, filtered and concentrated under vacuum to provide the crude title compound as a yellowish orange gum.

Step 2: 1-{3-Benzyl-5-[1-(8-hydroxy-[1,6]naphthyridin-7-yl) methanoyl]benzyl}pyrrolidin-2-one

7-{1 -[3-Benzyl-5-(2-oxo-pyrrolidin-1-ylmethyl)-phenyl]-methanoyl}-8-hydroxy-5H-[1,6]naphthyridine-6-carbamic acid benzyl ester was treated in a manner similar to Example 16 Step 2 to provide the title compound as a lyophillized yellow solid.

¹H NMR (500 MHz, CDCl₃) δ 9.3 (dd, 1H), 8.8 (s, 1H), 8.4 (dd, 1H), 7.98 (s, 1H), 7.97 (s, 1H), 7.8 (dd, 1H), 7.5 (s, 1H), 7.3 (m, 3H), 7.2 (m, 3H), 4.5 (s, 2H), 4.1 (s, 2H), 3.3 (t, 2H), 2.5 (t, 2H), 2.03 (p, 2H). ES MS M+1=438

### EXAMPLE 19

### N-{3-Benzyl-5-[1-(8-hydroxy-[1,6]naphthyridin-7-yl)methanoyl]benzyl}formamide

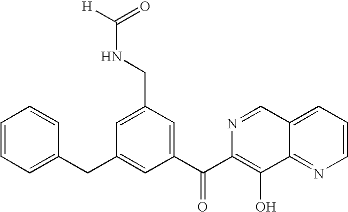

The title compound was prepared using a procedure similar to that described in Example 18, except that formamide was substituted for pyrrolidinone in Step 1 and in Step 2.the unoxidized intermediate was isolated by preparative HPLC and allowed to air oxidize prior to isolation of the final product by preparative HPLC.

¹H NMR (500 MHz, CDCl₃) δ 13.7 (brs, 1H), 9.3 (dd, 1H), 8.8 (s, 1H), 8.4 (dd, 1H), 8.3 (s, 0.8H), 8.23 (s, 0.2H), 8.20 (s, 0.2H), 8.01 (s, 1H),7.98 (s, 0.8H), 7.8 (dd, 1H), 7.5 (s, 1H), 7.35-7.2 (m, 5H), 6.6 (d,1H), 5.2 (s, 2H), 4.1 (s, 2H). ES MS M+1=398

### EXAMPLE 20

### N-{3-Benzyl-5-[1-(8-hydroxy-[1,6]naphthyridin-7-yl)methanoyl]benzyl}—N-methylformamide

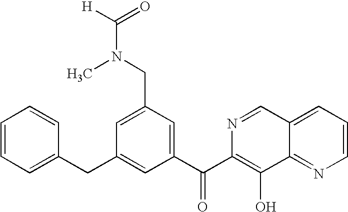

The title compound was prepared using a procedure similar to that described in Example 19, except that N-methylformamide is substituted for formamide in Step 1.

¹H NMR (500 MHz, CDCl₃) δ 13.7 (brs, 1H), 9.3 (m, 1H), 8.8 (s, 1H), 8.4 (dt, 1H), 8.33 (s, 0.5H), 8.2 (s, 0.5H), 8.02 (s, 0.5H), 7.97 (m, 1.5H),7.8 (p, 1H), 7.35-7.2 (m, 6H), 4.6 (s,1H), 4.5 (s, 1H), 4.09 (s, 1H) 4.1 (s, 1H), 2.9 (s, 1.5H), 2.85 (s, 1.5H). ES MS M+1=412

### EXAMPLE 21

### 1-(8—Hydroxy-[1,6]naphthyridin-7-yl)-1-(3-pyrazol-1-ylmethyl-5-pyridin-2-ylmethylphenyl)methanone (21)

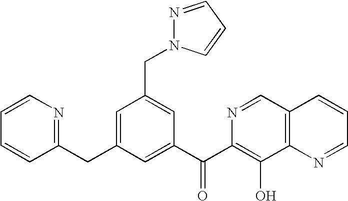 Step 1: tert-Butyl-(3,5-dibromobenzyloxy)dimethyl silane (21A)

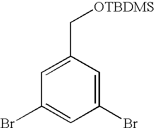

A solution of 3,5-dibromobenzyl alcohol (16.5 g, 62.0 mmol), imidazole (10.8 g, 158.7 mmol) and tert-butyldimethylchlorosilane (11.5 g, 76.3 mmol) in DMF (100 mL) was stirred over weekend at ambient temperature. The solvent was removed under vacuum. The residue was dissolved in diethyl ether and washed with water four times and brine once. The organic phase was dried over magnesium sulfate and concentrated to give 21A.
Step 2: [3—Bromo-5-(tert-butyl-dimethyl-silanyloxymethyl)-phenyl]-pyridin-2-yl-methanone (21B)

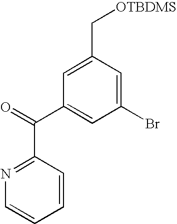

To a solution of 21A (23.5 g, 61.8 mmol) in anhydrous diethyl ether (200 mL) under argon was added n-butyllithium ( 2.5 M in hexanes, 26 mL, 65.0 mmol) dropwise at −78° C. After 1 h, a solution of pyridine-2-carboxylic acid methoxy-methyl-amide (10.8 g, 65.0 mmol) in diethyl ether (40 mL) was added. The reaction was allowed to warm slowly to ambient temperature overnight. The reaction mixture was treated with 1N HCl (50 mL) and extracted with diethyl ether three times. The combined organic phases were washed with brine, dried over sodium sulfate, filtered and concentrated. The residue was used for the next reaction without further purification.
Step 3: (3-Bromo-5-pyridin-2-ylmethyl-phenyl)-methanol (21C)

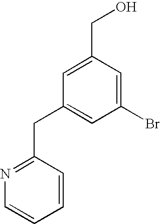

A mixture of 21B ( 61.8 mmol) and anhydrous hydrazine (33 mL) in ethyleneglycol (150 mL) was heated at 100° C. for 3h under argon. The excess hydrazine was evaporated in vacuo and the remain reaction mixture was co-evaporated with toluene once. The reaction mixture was then heated at 160° C. and potassium hydroxide (20 g, 356 mmol) was added portionwise. After 1 h, the reaction mixture was cooled to room temperature, neutralized with 1N HCl to pH 9 and extracted with chloroform five times. The combined organic phases were washed with brine, dried over sodium sulfate, filtered and concentrated under vacuum. The residue was purified by flash chromatography (ethyl acetate/chloroform) to give 21C.
Step 4: 1-(3-hydroxymethyl-5-pyridin-2-ylmethyl-phenyl)-ethanone (21D)

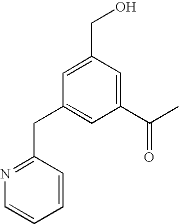

A seal tube was charged with 21C (3.05 g, 11.0 mmol), thallium acetate (3.62 g, 13.7 mmol), 1,3-bis(diphenylphosphino)propane (920 mg, 2.22 mmol), triethylamine (6.2 mL, 44.5 mmol) and DMF (30 mL). This mixture was purged with argon for 10 min. Palladium acetate (490 mg, 2.18 mmol) and butyl vinyl ether (7.2 mL, 55.6 mmol) were then added and the reaction mixture was stirred at 100° C. overnight. After cooling to room temperature, the reaction mixture was filtered through Celite. DMF was removed under vacuum. The residue was redissolved in THF (10 mL) and treated with 1N HCl (21 mL). After 2.5h, the reaction mixture was neutralized with 1N NaOH to pH 8 and extracted with chloroform four times. The combined organic layers were washed with brine, dried over sodium sulfate, filtered and concentrated. The residue was purified by flash chromatography (hexanes/ethyl acetate) to give 21D.
Step 5: (2—Chloro-pyridin-3-ylmethyl)-[2-(3-hydroxymethyl-5-pyridin-2-ylmethyl-phenyl)-2-oxo-ethyl]-carbamic acid benzyl ester (21E)

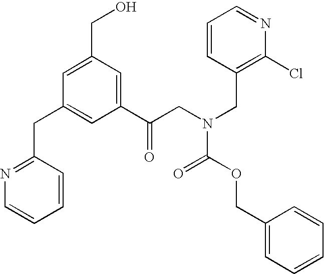

To a mixture of 21D (2.3 g, 9.53 mmol) and aluminum chloride (100 mg, 0.75 mmol) in 1,4-dioxane (20 niL) was added a solution of bromine (0.54 mL, 10.5 mmol) in 1,4-dioxane (15 mL) dropwise. After 30 min, the solvent was removed in vacuo. The residue was dissolved in DMF (10 mL) and this solution was cannulated into a solution of amine 7D (1.5 g, 10.6 mmol) and diisopropylethylamine (14 mL, 80.4 mmol) in DMF (30 mL). After 30 min, benzyl chloroformate (1.6 mL, 11.2 mmol) was added. The reaction mixture was stirred overnight. The solvent was removed under vacuum. The residue was partitioned between chloroform and brine. The organic layer was dried over sodium sulfate and concentrated. The residue was purified by flash chromatography (hexanes/ethyl acetate) to give 21E.
Step 6: 3-({Benzyloxycarbonyl-[2-(3-hydroxymethyl-5-pyridin-2-ylmethyl-phenyl)-2-oxo-ethyl]-amino}-methyl)-pyridine-2-carboxylic acid ethyl ester (21F)

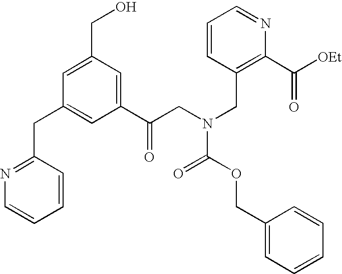

A mixture of 21E (1.25 g, 2.43 mmol), palladium acetate (110 mg, 0.49 mmol), 1,3-bis(diphenylphosphino)propane (220 mg, 0.53 mmol) and sodium acetate (220 mg, 2.68 mmol) in ethanol (40 mL) in a parr bomb flask was purged with argon for 10 min. The bomb was then pressurized with carbon monoxide to 300 psi and heated at 100° C. overnight. After cooling to room temperature, the reaction mixture was filtered through Celite and concentrated. The resultant residue was purified by flash chromatography (methanol/chloroform) to give 21F.
Step 7 8-hydroxy-7-(3-pyrazol-1-ylmethyl-5-pyridin-2-ylmethyl-benzoyl)-5H-[1,6]naphthyridine-6-carboxylic acid benzyl ester (21G)

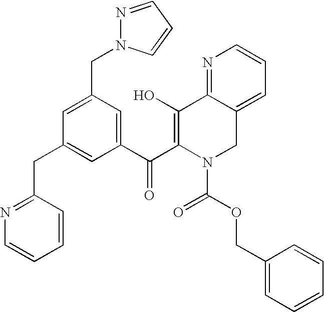

To a solution of 21F (180 mg, 0.325 mmol) in DMF (5 mL) at 0° C. was added diisopropylethylamine (0.11 mL, 0.62 mmol) and methanesulfonyl chloride (0.038 mL, 0.49 mmol) under argon and stirred for 40 min. Meanwhile, a separate flask, charged with sodium hydride (60% in mineral oil, 68 mg, 1.7 mmol) in DMF (5 mL) under argon, was added 1-pyrazole (78 mg, 1.15 mmol) and the reaction mixture was stirred for 15 min at ambient temperature then cooled to 0° C. The mesylate in the first flask was then cannulated to the second flask under argon at 0° C. After 1 h, the reaction mixture was treated with 1N HCl to pH 8 and extracted with chloroform four times. The combined organic phases were dried over sodium sulfate, filtered and concentrated. The residue was purified by preparative reverse phase HPLC to give 21G.

Step 8 1-(8—Hydroxy-[1,6]naphthyridin-7-yl)-1-(3-pyrazol-1-ylmethyl-5-pyridin-2-ylmethylphenyl)methanone (21)

To a solution of 21G (57 mg, 0.085 mmol) in dichloromethane (10 mL) was added boron tribromide (1.0 M in dichloromethane, 0.45 mL, 0.45 mmol) under argon at 0° C. After 30 min, methanol was added and the reaction mixture was evaporated to dryness. The residue was co-evaporated with methanol and chloroform saturated with ammonium three times. The resultant residue was then purified by preparative reverse phase HPLC to give 21.

¹H NMR (400 MHz, CD₃OD) δ 9.17 (dd, 1H), 8.84 (s, 1H), 8.67 (dd, 1H), 8.60 (dd, 1H), 8.32 (m, 1H), 8.04 (s, 1H), 7.95 (s, 1H), 7.89 (dd, 1H), 7.75-7.80 (m, 3H), 7.53 (d, 1H), 7.43 (s, 1H), 6.34 (t, 1H), 5.45 (s, 2H), 4.43 (s, 2H). ES MS M+1=422

### EXAMPLE 22

### 1-(8—Hydroxy-[1,6]naphthyridin-7-yl)-1-[3-(1,1-dioxo-isothiazolidin-2-ylmethyl)-5-pyridin-2-ylmethylphenyl]methanone (22) (L-870,349)

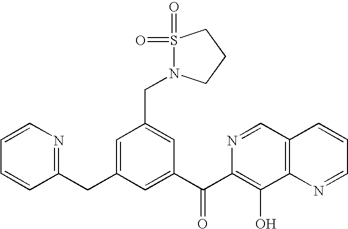

The title compound was prepared in a manner similar to that described in Example 21, except that 1-pyrazole was substituted with 3-chloro-propane-1-sulfonamide in step 7.

¹H NMR (400 MHz, CD₃OD) δ 9.17 (dd, 1H), 8.89 (s, 1H), 8.72 (d, 1H), 8.60 (dd, 1H), 8.43 (td, 1H), 8.14 (s, 1H), 8.04 (s, 1H), 7.82-7.92 (m, 3H), 7.63 (s, 1H), 4.53 (s, 2H), 4.28 (s, 2H), 3.16-3.30 (m, 4H), 2.34 (quintet, 2H). ES MS M+1=475

### EXAMPLE 23

### 1-(8—Hydroxy-[1,6]naphthyridin-7-yl)-1 -[3-(pyridin-2-one-1-ylmethyl)-5-pyridin-2-ylmethylphenyl]methanone (23)

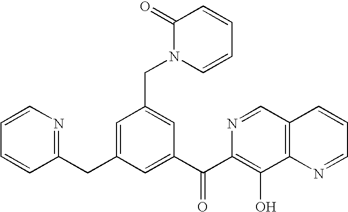

The title compound was prepared in a manner similar to that described in Example 21, except that 1-pyrazole was substituted with 2-pyridone in step 7.

¹H NMR (400 MHz, CD₃OD) δ 9.17 (dd, 1H), 8.84 (s, 1H), 8.74 (d, 1H), 8.60 (dd, 1H), 8.50 (t, 1H), 8.09 (s, 1H), 8.04 (s, 1H), 7.89-7.93 (m, 3H), 7.80 (dd, 1H), 7.60 (s, 1H), 7.55 (td, 1H), 6.58 (d, 1H), 6.43 (td, 1H), 5.30 (s, 2H), 4.53 (s, 2H). ES MS M+1=449

### EXAMPLE 24

### 1-(8—Hydroxy-[1,6]naphthyridin-7-yl)-1-[3-(piperidin-2-one-1-ylmethyl)-5-pyridin-2-ylmethylphenyl]methanone (24)

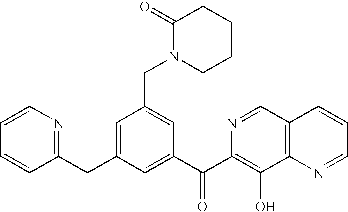

The title compound was prepared in a manner similar to that described in Example 21, except that 1-pyrazole was substituted with 2-piperidone in step 7.

¹H NMR (400 MHz, CD₃OD) δ 9.17 (dd, 1H), 8.89 (s, 1H), 8.74 (d, 1H), 8.62 (dd, 1H), 8.50 (td, 1H), 8.05 (s, 1H), 8.01 (s, 1H), 7.89-7.92 (m, 3H), 7.52 (s, 1H), 4.69 (s, 2H), 4.54 (s, 2H), 3.38 (m, 2H), 2.42 (m, 2H), 1.83 (m, 4H). ES MS M+1=453

### EXAMPLE 25

### 7-[1-(4-Benzylpyridin-2-yl)methanoy]]-8-hydroxy-6H-[1,6]naphthyridin-5-one (25)

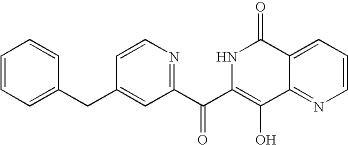 Step 1: 4-benzylpyridine N-oxide (25A)

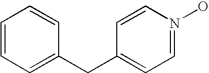

To a solution of 4 benzylpyridine (15.9 g) in glacial acetic acid (90 mL), was added 35% aqueous hydrogen peroxide and the mixture stirred in an 85 C. oil bath overnight. After cooling to ambient temperature the mixture was treated slowly with saturated aqueous sodium bicarbonate to a pH of ˜8 and extracted with methylene chloride (3×). The combined organic extracts were washed with brine, dried over anydrous magnesium sulfate, filtered and concentrated under vacuum to the N-oxide, a tan solid.
Step 2: 4-Benzylpyridine-2-carbonitrile (25B)

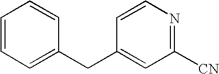

To a solution of 4-benzylpyridine N-oxide (28 g) and triethylamine (31.6 mL)in anhydrous acetonitrile (160 mL), under an atmosphere of nitrogen, was added trimethylsilylcyanide (50 mL) over a period of 15 minutes. The mixture was heated to reflux and stirred overnight. After cooling to ambient temperature, the mixture was diluted with methylene chloride and washed with saturated aqueous sodium bicarbonate (2×), dried over anhydrous sodium sulfate, filtered and concentrated under vacuum to adark brown oil. Column chromatography on silica gel with 30% ethyl acetate in hexane provided the title nitrile as a yellow oil.
Step 3: 1-(4-Benzylpyridin-2-yl) ethanone. (25C)

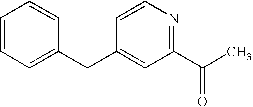

To a solution of 4-benzylpyridine-2-carbonitrile (10.2 g) in anhydrous ether (110 mL), under an atmosphere of nitrogen, was added a 3 M solution of methyl magnesium iodide in ether (21 mL), over 10 minutes. The resulting slurry was stirred 2 hours at ambient temperature, then cooled in an ice bath and treated slowly with 1M aqueous HCl (200 mL). The mixture was allowed to warm to ambient temperature over 1 hour then neutralized with 1M aqueous sodium hydroxide to a pH of 7 and extracted with methylene chloride (3×). The combined organic extracts were dried over anhydrous sodium sulfate, filtered and concentrated under vacuum to a brown oil. Column chromatography on silica gel with 20% ethyl acetate in hexane provided the title ketone as a yellow oil.
Step 4: 1-(4-Benzylpyridin-2-yl)-2-bromoethanone (25D)

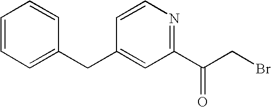

To a mixture of 1-(6-benzylpyridin-2-yl) ethanone (5.3 g) and sodium bromate (1.26 g) in glacial acetic acid (21.5 mL), was added a solution of 48% aqueous HBr (11.4 mL) over 15 minutes. The reaction was placed in an oil bath which was heated slowly over 30 minutes to 95 C. and held at that temperature for 30 minutes. After cooling to ambient temperature, the mixture was poured into a mixture of saturated aqueous sodium bicarbonate and ice. Additional saturated aqueous sodium bicarbonate was added to bring the pH to ˜7-8 and the mixture extracted with ethyl acetate (3×). The combined organic extracts were washed with brine, dried over anhydrous sodium sulfate, filtered and concentrated under vacuum to a brown oil. Column chromatography on silica gel with 7.5% ethyl acetate in hexane provided the title bromoketone as a pale brown oil.
Step 5: Pyrrolo[3,4-b]pyridine-5,7-dione. 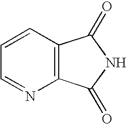
A mixture of quinolinic anhydride (40 g) and acetamide (40 g) in acetic anhydride (40 mL) was heated in a 140 C. oil bath for 2 hours. After cooling to 0 C. in an ice bath, the precipitated solid was isolated by filtration, washed with ice cold methanol (2×), air dried, then dried under vacuum to provide the title compound as a grey solid.
Step 6: 6-[2-(4-Benzylpyridin-2-yl)-2-oxoethyl]pyrrolo[3,4-b]pyridine-5,7-dione (25E)

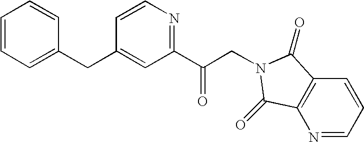

To a solution of 1-(4-benzylpyridin-2-yl)-2-bromoethanone (0.6 g) in anhydrous DMF (10 mL) under a nitrogen atmosphere, was added pyrrolo[3,4-b]pyridine-5,7-dione (0.37 g) and cesium carbonate (0.81 g). The mixture was stirred at ambient temperature for 2 hours and poured into ice water. The insoluble solid was isolated by filtration, washed with deionized water (3×), and dried under vacuum to provide the title compound as a light tan solid.

Step 7: 7-[1-(4-Benzylpyridin-2-yl)methanoyl]-8-hydroxy-6H-[1,6]naphthyridin-5-one (25)

To a hot (90-100 C.) solution of sodium methoxide, prepared by dissolving sodium metal (0.09 g) in anhydrous methanol (3 mL), was added 6-[2-(4-benzylpyridin-2-yl)-2-oxoethyl]pyrrolo[3,4-b]pyridine-5,7-dione (0.35 g) in one portion. Heating was continued for 45 minutes and then the slurry allowed to cool to ambient temperature at which time it was acidified with 10% aqueous oxalic acid. The resulting red solid was isolated by filtration, washed with several portions of deionized water and dried under vacuum to afford a red solid which was a mixture of regioisomers. Separation and purification of the regioisomers was accomplished by preparative HPLC with Waters PrepPak C18 cartridges and acetonitrile/ water with trifluoroacetic acid modifier as the mobile phase. Lyophilization of the earlier eluting fractions provided the title compound as a red lyophilized solid.

¹H NMR (500 MHz, CDCl₃) δ 11.0 (brs, 1H), 9.2 (dd, 1H), 8.8 (dd, 1H), 8.6 (d, 1H), 8.4 (s, 1H), 7.7 (dd, 1H), 7.5 (dd, 1H), 7.4- 7.2 (m, 5H), 4.2 (s, 2H.). ES MS M+1=358

### EXAMPLE 26

Oral Composition

As a specific embodiment of an oral composition of a compound of this invention, 50 mg of compound of Example 14 is formulated with sufficient finely divided lactose to provide a total amount of 580 to 590 mg to fill a size 0 hard gelatin capsule.

### EXAMPLE 27

### HIV Integrase Assay: Strand Transfer Catalyzed by Recombinant Integrase

Assays for the strand transfer activity of integrase were conducted in accordance with Wolfe, A. L. et al., J. Virol. 1996, 70: 1424-1432, for recombinant integrase, except that: (i) the assays used preassembled integrase strand transfer complexes; (ii) the strand transfer reaction was performed in the presence of inhibitor in 2.5 mM MgCl₂ using 0.5 to 5 nM of a 3′ FITC labeled target DNA substrate (SEQ. ID. NO: 1 and SEQ. ID. NO: 2)

| | | |
|---|---|---|
| 5′ | TGA CCA AGG GCT AAT TCA CT fitc | 3′ |
| 3′ fitc | ACT GGT TCC CGA TTA AGT GA | 5′; |

and (iii) strand transfer products were detected using an alkaline phosphatase conjugated anti—FITC antibody and a chemiluminescent alkaline phosphatase substrate. Representative compounds tested in the integrase assay demonstrated IC₅₀'s of less than about 100 micromolar.

Further description on conducting the assay using preassembled complexes is found in Hazuda et al., J. Virol. 1997, 71: 7005-7011; Hazuda et al., Drug Design and Discovery 1997, 15: 17-24; and Hazuda et al., Science 2000, 287: 646-650.

### EXAMPLE 28

Assay for Inhibition of HIV Replication

Assays for the inhibition of acute HIV infection of T-lymphoid cells were conducted in accordance with Vacca, J. P. et al., (1994), Proc. Natl. Acad. Sci. USA 91, 4096. Representative compounds tested in the present assay demonstrated IC₉₅'s of less than about 10 micromolar.

While the foregoing specification teaches the principles of the present invention, with examples provided for the purpose of illustration, the practice of the invention encompasses all of the usual variations, adaptations and/or modifications that come within the scope of the following claims.

1. A compound of Formula (I): 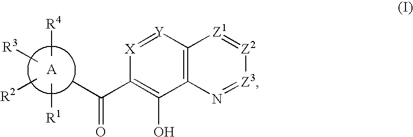
wherein A is
(1) phenyl,
(2) phenyl fused to a carbocycle to form a fused carbocyclic ring system; or
(3) heterocycle containing one or more heteroatoms selected from nitrogen, oxygen and sulfur and a balance of carbon atoms, with at least one of the ring atoms being carbon;
A is connected by a ring carbon to the exocyclic carbonyl, and is substituted by R¹, R², R³, and R⁴;
X is N or C-Q¹;
Y is N or C-Q², provided that X and Y are not both N;
Z¹ is N or C-Q³;
Z² is N or C-Q⁴;
Z³ is N or CH;
each of Q¹, Q², Q³, and Q⁴ is independently
(1) —H,
(2) —C1 -6 alkyl,
(3) —C₁₋₆ fluoroalkyl,
(4) —OH,
(5) —O—C₁₋₆ alkyl,
(6) —O—C₁₋₆ fluoroalkyl,
(7) halo,
(8) —CN,
(9) —C₁₋₆ alkyl-OR^{a},
(10) —C₀₋₆ alkyl-C(═O)R^{a},
(11) —C₀₋₆ alkyl-CO₂R^{a},
(12) —C₀₋₆ alkyl-SR^{a},
(13) —N(R^{a})₂,
(14) —C₁₋₆ alkyl —N(R^{a})₂,
(15) —C₀₋₆ alkyl-C(═O)N(R^{a})₂,
(16) —C₁₋₆ alkyl-N(R^{a})—C(R^{a})═O,
(17) —SO₂R^{a},
(18) —N(R^{a})SO₂R^{a},
(19) —C₂₋₅ alkynyl,
(20) —C₂₋₅ alkynyl-CH₂N(R^{a})₂,
(21) —C₂₋₅ alkynyl-CH₂OR^{a},
(22) 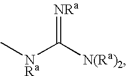
(23) —N(R^{a})—C₁₋₆ alkyl-SR^{a},
(24) —N(R^{a})—C₁₋₆ alkyl-OR^{a},
(25) —N(R^{a})—C₁₋₆ alkyl-N(R^{a})₂,
(26) —N(R^{a})—C₁₋₆ alkyl-N(R^{a})—C(R^{a})═O,
(27) —R^{k},
(28) —C₁₋₆ alkyl substituted with R^{k},
(29) —C₁₋₆ fluoroalkyl substituted with R^{k},
(30) —C₂₋₅ alkenyl-R^{k},
(31) —C₂₋₅ alkynyl-R^{k},
(32) —O—R^{k},
(33) —O—C₁₋₄ alkyl-R^{k},
(34) —S(O)ₙ—R^{k},
(35) —S(O)ₙ—C₁₋₄ alkyl-R^{k},
(36) —O—C₁₋₆ alkyl-OR^{k},
(37) —O—C₁₋₆ alkyl-O—C₁₋₄ alkyl-R^{k},
(38) —O—C₁₋₆ alkyl-SR^{k},
(39) —N(R^{c})—R^{k},
(40) —N(R^{c})—C₁₋₆ alkyl substituted with one or two R^{k}groups;
(41) —N(R^{c})—C₁₋₆ alkyl-OR^{k},
(42) —C(═O)N—C₁₋₆ alkyl-R^{k}, or
(43) —C₂₋₅ alkynyl-CH₂S(O)ₙ—R^{a};
each of R¹ and R² is independently:
(1) —H,
(2) —C₁₋₆ alkyl,
(3) —C₁₋₆ fluoroalkyl,
(4) —O—C₁₋₆ alkyl,
(5) —O—C 1-6 fluoroalkyl,
(6) —OH,
(7) halo,
(8) —NO₂,
(9) —CN,
(10) —C₁₋₆ alkyl-OR^{a},
(11) —C₀₋₆ alkyl-C(═O)R^{a},
(12) —C₀₋₆ alkylCO₂R^{a},
(13) —C₀₋₆ alkyl-SR^{a},
(14) —N(R^{a})₂,
(15) —C₁₋₆ alkyl-N(R^{a})₂,
(16) —C₀₋₆ alkyl-C(═O)N(R^{a})₂,
(17) —C₁₋₆ alkyl-N(R^{a})—C(R^{a})═O,
(18) —SO₂R^{a},
(19) —N(R^{a})SO₂R^{a},
(20) —C₂₋₅ alkenyl,
(21) —O—C₁₋₆ alkyl-OR^{a},
(22) —O—C₁₋₆ alkyl-SR^{a},
(23) —O—C₁₋₆ alkyl-NH—CO₂R^{a},
(24) —O—C₂₋₆ alkyl-N(R^{a})₂,
(25) —N(R^{a})—C₁₋₆ alkyl-SR^{a},
(26) —N(R^{a})—C_{1 6} alkyl-OR^{a},
(27) —N(R^{a})—C₁₋₆ alkyl-N(R^{a})₂,
(28) —N(R^{a})-C₁₋₆ alkyl-N(R^{a})—C(R^{a})═O,
(29) —R^{k},
(30) —C16 alkyl substituted with 1 or 2 R^{k}groups,
(31) —C16 fluoroalkyl substituted with 1 or 2 R^{k}groups,
(32) —C2-5 alkenyl-R^{k},
(33) —C2-5 alkynyl-R^{k},
(34) —OR^{k},
(35) -0—C₁₋₄ alkyl-R^{k},
(36) —S(O)ₙ—R^{k},
(37) —S(O)ₙ—C₁₋₄ alkyl-R^{k},
(38) —O—C₁₋₆ alkyl-OR^{k},
(39) —O—C₁₋₆ alkyl-O—C14 alkyl-R^{k},
(40) —O—C₁₋₆ alkyl-SR^{k},
(41) —C₁₋₆ alkyl (OR^{b})(R^{k}),
(42) —C₁₋₆ alkyl (OR^{b})(—C₁₋₄ alkyl-R^{k}),
(43) —C₀₋₆ alkyl-N(R^{b})(R^{k}),
(44) —C₀₋₆ alkyl-N(R^{b})(—C₁₋₄ alkyl-R^{k}),
(45) —C₁₋₆ alkyl S(O)ₙ—^{k},
(46) —C₁₋₆ alkyl S(O)ₙ—C₁₋₄ alkyl-R^{k},
(47) —C₀₋₆ alkyl C(O)—R^{k}, or
(48) —C₀₋₆ alkyl C(O)—C₁₋₄ alkyl-R^{k};
each of R³ and R⁴ is independently
(1) —H,
(2) halo,
(3) —CN,
(4) —NO₂,
(5) —OH,
(6) C₁₋₆ alkyl,
(7) C₁₋₆ fluoroalkyl,
(8) —O—C₁₋₆ alkyl,
(9) —O—C₁₋₆ fluoroalkyl,
(10) —C₁₋₆ alkyl-OR^{a},
(11) —C₀₋₆ alkyl-C(═O)R^{a},
(12) —C₀₋₆ alkyl-CO₂R^{a},
(13) —C₀₋₆ alkyl-SR^{a},
(14) —N(R^{a})₂,
(15) —C₁₋₆ alkyl-N(R^{a})₂,
(16) —C₀₋₆ alkyl-C(═O)N(R^{a})₂,
(17) —SO₂R^{a},
(18) —N(R^{a})SO₂R^{a},
(19) —C₂₋₅ alkenyl,
(20) —O—C₁₋₆ alkyl-OR^{a},
(21) —O—C₁₋₆ alkyl-SR^{a},
(22) —O—C₁₋₆ alkyl-NH—CO₂R^{a},
(23) —O—C₂₋₆ alkyl-N(R^{a})₂, or
(24) oxo;
each R^{a} is independently —H, —C₁₋₆ alkyl, or —C₁₋₆ fluoroalkyl;
each R^{b} is independently:
(1) —H,
(2) —C₁₋₄ alkyl,
(3) —C₁₋₄ fluoroalkyl,
(4) —R^{k},
(5) —C₂₋₃ alkenyl,
(6) —C₁₋₄ alkyl-R^{k},
(7) —C₂₋₃ alkenyl-R^{k},
(8) —S(O)ₙ—R^{k}, or
(9) —C(O)—R^{k};
each R^{c} is independently
(1) —H,
(2) —C₁₋₆ alkyl,
(3) —C₁₋₆ alkyl substituted with —N(R^{a})₂, or
(4) —C₁₋₄ alkyl-aryl, wherein aryl is optionally substituted with 1 to 5 substituents independently selected from halogen, C₁₋₆ alkyl, C₁₋₆ fluoroalkyl, —O—C₁₋₆ alkyl, —O—C₁₋₆ fluoroalkyl, —S—C₁₋₆ alkyl, —CN, and —OH;
each R^{k} is independently carbocycle or heterocycle, wherein either the carbocycle or heterocycle is unsubstituted or substituted with from 1 to 5 substituents each of which is independently selected from
(a) halogen,
(b) C₁₋₆ alkyl,
(c) C₁₋₆ fluoroalkyl,
(d) —O—C₁₋₆ alkyl,
(e) —O—C₁₋₆ fluoroalkyl,
(f) —S-C₁₋₆ alkyl,
(g) —CN,
(h) —OH,
(i) oxo,
(j) —(CH₂)₀₋₃C(═O)N(R^{a})₂,
(k) —(CH₂)₀₋₃C(═O)R^{a},
(1) —N(R^{a})—C(═O)R^{a},
(m) —N(R^{a})—C(═O)OR^{a},
(n) —(CH₂)₁₋₃N(R^{a})—C(═O)R^{a},
(o) —N(R^{a})₂,
(p) —C₁₋₆ alkyl-N(R^{a})₂,
(q) aryl,
(r) aryloxy-,
(s) —C₁₋₄ alkyl substituted with aryl,
(t) heteromonocycle,
(u) —C₁₋₄ alkyl substituted with a heteromonocycle,
(v) heteromonocyclylcarbonyl-C₀₋₆ alkyl-,
(w) N-heteromonocyclyl-N—C₁₋₆ alkyl-amino-;
wherein the aryl group in (q) aryl, (r) aryloxy, and (s) —C₁₋₄ alkyl substituted with aryl, is optionally substituted with from 1 to 3 substituents independently selected from halogen, C₁₋₆ alkyl, —O—C₁₋₆ alkyl, C₁₋₆ alkyl substituted with N(R^{a})₂, C₁₋₆ fluoroalkyl, and —OH; and
wherein the heteromonocyclyl group in (t) heteromonocycle, (u) —C₁₋₄ alkyl substituted with a heteromonocycle, (v) heteromonocyclyl-carbonyl-C₀₋₆ alkyl-, and (w) N-heteromonocyclyl-N—C₁₋₆ alkyl-amino- is optionally substituted with from 1 to 3 substituents independently selected from halogen, C₁₋₆ alkyl, —O—C₁₋₆ alkyl, C₁₋₆ fluoroalkyl, oxo, and —OH; and
each n is independently an integer equal to 0, 1 or 2;
and provided that:
(i) when A is phenyl, X is CH, Y is CH, and Z¹=Z²=Z³=CH, then at least one of R¹, R², R³, and R⁴ is not —H;
(ii) when A is phenyl, X is CH, Y is CQ² wherein Q² is halo or —C₁₋₆ alkyl or phenyl optionally substituted with halo or —C 1-6 alkyl or benzyl optionally substituted with halo or —C -6 alkyl, Z¹=Z²=Z³=CH, and all but one of R¹, R², R³ and R⁴ are independently —H, halo or —C16 alkyl, then the other of R¹, R², R³ and R⁴ is not —H, halo or —C16 alkyl;
(iii) when A is phenyl, X is CH, Y is CH, Z¹=Z²=Z³=CH, and one of R¹, R², R³, and R⁴ is —CO₂R^{a}, then at least one of the others of R¹, R², R³, and R⁴ is not —H;
(iv) when A is phenyl, X is N, Y is C—OH, and Z¹=Z²=Z³=CH, then at least one of R¹, R², R³, and R⁴ is not —H; and
(v) when A is phenyl, X is CH, Y is CH, Z¹ is CQ³, and Z²=Z³=CH, then either (v-a) Q³ is not unsubstituted or substituted benzyl or (v-b) at least one of R¹, R², R³, and R⁴ is not —H;
or a pharmaceutically acceptable salt thereof.

2. The compound according to claim 1, wherein
X is N;
Y is C-Q²;
Z¹ is C-Q³;
Z² is C-Q⁴;
Z³ is CH;
Q² is
(1) —H,
(2) —C₁₋₆ alkyl,
(3) —C₁₋₆ fluoroalkyl,
(4) —OH,
(5) —O—C₁₋₆ alkyl,
(6) —O—C₁₋₆ fluoroalkyl,
(7) halo,
(8) —CN,
(9) —C₁₋₆ alkyl-OR^{a},
(10) —C₀₋₆ alkyl-C(═O)R^{a},
(11) —C₀₋₆ alkyl-CO₂R^{a},
(12) —C₀₋₆ alkyl-SR^{a},
(13) —N(R^{a})₂,
(14) —C₁₋₆ alkyl —N(R^{a})₂,
(15) —C₀₋₆ alkyl-C(═O)N(R^{a})₂,
(16) —C₁₋₆ alkyl-N(R^{a})—C(R^{a})═O,
(17) —SO₂R^{a},
(18) —N(R^{a})SO₂R^{a},
(19) —C₂₋₅ alkynyl,
(20) —C₂₋₅ alkynyl-CH₂N(R^{a})₂,
(21) —C₂₋₅ alkynyl-CH₂OR^{a},
(22) 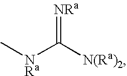
(23) —N(R^{a})—C₁₋₆ alkyl-SR^{a},
(24) —N(R^{a})—C₁₋₆ alkyl-OR^{a},
(25) —N(R^{a})—C₁₋₆ alkyl-N(R^{a})₂,
(26) —N(R^{a})—C₁₋₆ alkyl-N(R^{a})—C(R^{a})═O,
(27) —R^{k},
(28) —C₁₋₆ alkyl substituted with R^{k},
(29) —C₁₋₆ fluoroalkyl substituted with R^{k},
(30) —C₂₋₅ alkenyl-R^{k},
(31) —C₂₋₅ alkynyl-R^{k},
(32) —O—R^{k},
(33) —O—C₁₋₄ alkyl-R^{k},
(34) —S(O)ₙ—R^{k},
(35) —S(O)ₙ—C₁₋₄ alkyl-R^{k},
(36) —O—C₁₋₆ alkyl-OR^{k},
(37) —O—C₁₋₆ alkyl-O—C₁₋₄ alkyl-R^{k},
(38) —O—C₁₋₆ alkyl-SR^{k},
(39) —N(R^{c})—R^{k},
(40) —N(R^{c})—C₁₋₄ alkyl substituted with one or two R^{k}groups,
(41) —N(R^{c})—C₁₋₆ alkyl-OR^{k},
(42) —C(═O)N—C₁₋₆ alkyl-R^{k}, or
(43) —C₂₋₅ alkynyl-CH₂S(O)ₙ—R^{a}; and
each of Q³ and Q⁴:
(1) —H,
(2) —C₁₋₆ alkyl,
(3) —C₁₋₆ fluoroalkyl,
(4) —OH,
(5) —O—C₁₋₆ alkyl,
(6) —O—C₁₋₆ fluoroalkyl,
(7) halo,
(8) —CN,
(9) —C₁₋₆ alkyl-OR^{a},
(10) —C₀₋₆ alkyl-C(═O)R^{a},
(11) —C₀₋₆ alkyl-CO₂R^{a},
(12) —SR^{a},
(13) —N(R^{a})₂,
(14) —C₁₋₆ alkyl —N(R^{a})₂,
(15) —C₀₋₆ alkyl-C(═O)N(R^{a})₂,
(16) —SO₂R^{a},
(17) —N(R^{a})SO₂R^{a},
(18) —R^{k}, or
(19) —C₁₋₆ alkyl substituted with R^{k};
and provided that when A is phenyl, Y is C—OH, and Z¹ are Z² are both CH, then at least one of R¹, R², R³, and R⁴ is not —H;
or a pharmaceutically acceptable salt thereof.

3. The compound according to claim 2, wherein Q³ and Q⁴ are both —H;
and provided that when A is phenyl, Y is C—OH, then at least one of R¹, R², R³, and R⁴ is not —H;
or a pharmaceutically acceptable salt thereof.

4. The compound according to claim 1, which is a compound of Formula (II): 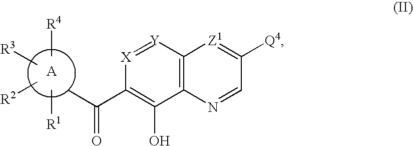
wherein
A is
(1) phenyl,
(2) a fused carbocyclic ring system selected from the group consisting of 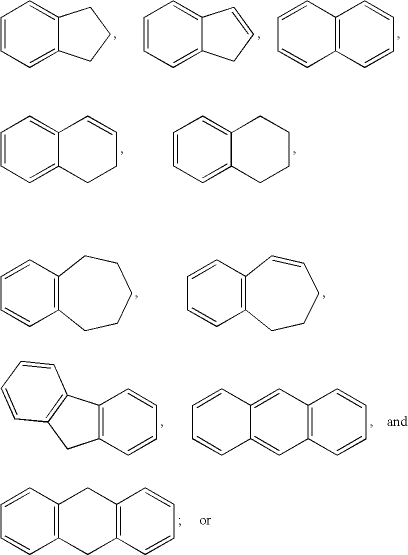
(3) a 5- or 6-membered saturated or unsaturated monocylic heterocycle which contains from 1 to 4 nitrogen atoms, from zero to 2 heteroatoms selected from oxygen and sulfur, and a balance of carbon atoms, with at least one of the ring atoms being carbon;
A is connected by a ring carbon to the exocyclic carbonyl, and is substituted by R¹, R², R³, and R⁴;
X is N or C-Q¹;
Y is N or C-Q², provided that X and Y are not both N;
Z¹ is N or C-Q³;
Q¹ is —H or —C₁₋₄ alkyl;
Q² is
(1) —H,
(2) —C₁₋₄ alkyl,
(3) —C₁₋₄ fluoroalkyl,
(4) —O—C₁₋₄ alkyl,
(5) —O—C₁₋₄ fluoroalkyl,
(6) —OH,
(7) halo,
(8) —CN,
(9) —C₁₋₄ alkyl-OR^{a},
(10) —(CH₂)₀₋₂C(═O)R^{a},
(11) —(CH₂)₀₋₂CO₂R^{a},
(12) —(CH₂)₀₋₂SR^{a},
(13) —N(R^{a})₂,
(14) —C₁₋₄ alkyl —N(R^{a})₂,
(15) —(CH₂)₀₋₂C(═O)N(R^{a})₂,
(16) —SO₂R^{a},
(17) —N(R^{a})SO₂R^{a},
(18) —C₂₋₃ alkynyl,
(19) —C≡C—CH₂N(R^{a})₂,
(20) —C≡C—CH₂OR^{a},
(21) —N(R^{a})—C₁₋₄ alkyl-SR^{a},
(22) —N(R^{a})—C₁₋₄ alkyl-OR^{a},
(23) —N(R^{a})—C₁₋₄ alkyl-N(R^{a})₂,
(24) —N(R^{a})—C₁₋₄ alkyl-N(R^{a})—C(R^{a})═O,
(25) —R^{k},
(26) —C₁₋₄ alkyl substituted with R^{k},
(27) —C₁₋₄ fluoroalkyl substituted with R^{k},
(28) —C₂₋₅ alkenyl-R^{k},
(29) —C₂₋₅ alkynyl-R^{k},
(30) —O—R^{k},
(31) —O—C₁₋₄ alkyl-R^{k},
(32) —S(O)ₙ—R^{k},
(33) —N(R^{c})—R^{k},
(34) —N(R^{c})—C₁₋₄ alkyl substituted with one or two R^{k}groups,
(35) —N(R^{c})—C₁₋₄ alkyl-OR^{k},
(36) —C(═O)N—C₁₋₄ alkyl-R^{k},
(37) —C≡C—CH₂SR^{a}, or
(38) —C≡C—CH₂SO₂R^{a};
Q³ is
(1) —H,
(2) —C₁₋₄ alkyl,
(3) —C₁₋₄ fluoroalkyl,
(4) —O—C₁₋₄ alkyl,
(5) —O—C₁₋₄ fluoroalkyl,
(6) halo selected from —F, —Cl, and —Br,
(7) —CN,
(8) —C₁₋₄ alkyl-OR^{a}, or
(9) —C₁₋₄ alkyl substituted with R^{k};
Q⁴ is:
(1) —H,
(2) —C₁₋₄ alkyl,
(3) —C₁₋₄ fluoroalkyl,
(4) —O—C₁₋₄ alkyl,
(5) —O—C₁₋₄ fluoroalkyl,
(6) halo selected from —F, —Cl, and —Br,
(7) —CN,
(8) —C₁₋₆ alkyl-OR^{a},
(9) —N(R^{a})₂, or
(10) —C₁₋₆ alkyl —N(R^{a})₂;
each of R¹ and R² is independently:
(1) —H,
(2) —C₁₋₄ alkyl,
(3) —C₁₋₄ fluoroalkyl,
(4) —O—C₁₋₄ alkyl,
(5) —O—C₁₋₄ fluoroalkyl,
(6) —OH,
(7) halo,
(8) —CN,
(9) —C₁₋₄ alkyl-OR^{a},
(10) —(CH₂)_{0- 2}C(═O)R^{a},
(11) —(CH₂)₀₋₂CO₂R^{a},
(12) —(CH₂)₀₋₂SR^{a},
(13) —N(R^{a})₂,
(14) —C₁₋₄ alkyl-N(R^{a})₂,
(15) —(CH₂)₀₋₂C(═O)N(R^{a})₂,
(16) —C₁₋₄ alkyl-N(R^{a})—C(R^{a})═O,
(17) —SO₂R^{a},
(18) —N(R^{a})SO₂R^{a},
(19) —O—C₁₋₄ alkyl-OR^{a},
(20) —O—C₁₋₄ alkyl-SR^{a},
(21) —O—C₁₋₄ alkyl-NH—CO₂R^{a},
(22) —O—C₂₋₄ alkyl-N(R^{a})₂,
(23) —N(R^{a})—C₁₋₄ alkyl-SR^{a},
(24) —N(R^{a})—C₁₋₄ alkyl-OR^{a},
(25) —N(R^{a})—C₁₋₄ alkyl-N(R^{a})₂,
(26) —N(R^{a})—C₁₋₄ alkyl-N(R^{a})—C(R^{a})═O,
(27) —R^{k},
(28) —C₁₋₄ alkyl substituted with 1 or 2 R^{k} groups,
(29) —C₁₋₄ fluoroalkyl substituted with 1 or 2 R^{k} groups,
(30) —O—R^{k},
(31) —O—C₁₋₄ alkyl-R^{k},
(32) —S(O)ₙ—R^{k},
(33) —S(O)ₙ—C₁₋₄ alkyl-R^{k},
(34) —O—C₁₋₄ alkyl-OR^{k},
(35) —O—C₁₋₄ alkyl-O—C₁₋₄ alkyl-R^{k},
(36) —O—C₁₋₄ alkyl-SR^{k}, or
(37) —C₀₋₄ alkyl-N(R^{b})(R^{k});
each of R³ and R⁴ is independently
(1) —H,
(2) halo,
(3) —CN,
(4) —OH,
(5) C₁₋₄ alkyl,
(6) C₁₋₄ fluoroalkyl,
(7) —O—C₁₋₄ alkyl,
(8) —O—C₁₋₄ fluoroalkyl,
(9) —C₁₋₄ alkyl-OR^{a},
(10) —O—C₁₋₄ alkyl-OR^{a},
(11) —O—C₁₋₄ alkyl-SR^{a},
(12) —O—C₁₋₄ alkyl-NH—CO₂R^{a}, or
(13) —O—C₂₋₄ alkyl-N(R^{a})₂;
each R^{a} is independently —H or —C₁₋₄ alkyl;
each R^{b} is independently:
(1) —H,
(2) —C₁₋₄ alkyl,
(3) —C₁₋₄ fluoroalkyl,
(4) —R^{k},
(5) —C₁₋₄ alkyl-R^{k},
(6) —S(O)ₙ—R^{k}, or
(7) —C(═O)—R^{k};
each R^{c} is independently
(1) —H,
(2) —C₁₋₄ alkyl,
(3) —C₁₋₄ alkyl substituted with —N(R^{a})₂, or
(4) —C₁₋₄ alkyl-phenyl, wherein the phenyl is optionally substituted with 1 to 3 substituents independently selected from halogen, C₁₋₄ alkyl, C₁₋₄ fluoroalkyl, —O—C₁₋₄ alkyl, —O—C₁₋₄ fluoroalkyl, —S—C₁₋₄ alkyl, —CN, and —OH;
each R^{k} is independently:
(1) aryl selected from phenyl and naphthyl, wherein aryl is unsubstituted or substituted with from 1 to 5 substituents independently selected from:
(a) halogen,
(b) C₁₋₆ alkyl,
(c) C₁₋₆ fluoroalkyl,
(d) —O—C₁₋₆ alkyl,
(e) —O—C₁₋₆ fluoroalkyl,
(f) phenyl,
(g) —S—C₁₋₆ alkyl,
(h) —CN,
(i) —OH,
(j) phenyloxy, unsubstituted or substituted with from 1 to 3 substituents independently selected from:
(i) halogen,
(ii) C₁₋₆ alkyl,
(iii) C₁₋₆ fluoroalkyl, and
(iv) —OH,
(k) —N(R^{a})₂,
(l) —C₁₋₆ alkyl-N(R^{a})₂,
(m) R^{t},
(p) —(CH₂)₀₋₃C(═O)N(R^{a})₂, and
(q) —(CH₂)₀₋₃C(═O)R^{a};
(2) —C₃₋₇ cycloalkyl, unsubstituted or substituted with from 1 to 3 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,,
(f) —CN,
(h) phenyl, and
(j) —OH;
(3) —C₃₋₇ cycloalkyl fused with a phenyl ring, unsubstituted or substituted with from 1 to 5 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,
(f) —CN, and
(g) —OH;
(4) a 5- or 6- membered heteroaromatic ring containing from 1 to 4 heteroatoms independently selected from oxygen, nitrogen and sulfur, wherein the heteroaromatic ring is unsubstituted or substituted on nitrogen or carbon with from 1 to 5 substituents independently selected from:
(a) halogen,
(b) C₁₋₆ alkyl,
(c) C₁₋₆ fluoroalkyl,
(d) —O—C₁₋₆ alkyl,
(e) —O—C₁₋₆ fluoroalkyl,
(f) phenyl,
(g) —S—C₁₋₆ alkyl,
(h) —CN,
(i) —OH,
(j) phenyloxy, unsubstituted or substituted with from 1 to 3 substituents independently selected from:
(i) halogen,
(ii) C₁₋₆ alkyl,
(iii) C₁₋₆ fluoroalkyl, and
(iv) —OH,
(k) —N(R^{a})₂,
(l) —C₁₋₆ alkyl-N(R^{a})₂,
(m) R^{t},
(n) oxo,
(o) —(CH₂)₀₋₃C(═O)N(R^{a})₂, and
(p) —(CH₂)₀₋₃C(═O)R^{a};
(5) a 5- or 6- membered saturated heterocyclic ring containing 1 or 2 heteroatoms independently selected from oxygen, nitrogen and sulfur, wherein the heterocyclic ring is unsubstituted or substituted with from 1 to 4 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,
(f) —CN,
(g) oxo,
(h) phenyl,
(i) benzyl,
(j) phenylethyl,
(k) —OH,
(l) —(CH₂)₀₋₃C(═O)N(R^{a})₂,
(m) —(CH₂)₀₋₃C(═O)R^{a},
(n) —N(R^{a})—C(═O)R^{a},
(o) —N(R^{a})—C(═O)OR^{a},
(p) —(CH₂)₁₋₃N(R^{a})—C(═O)R^{a},
(q) —N(R^{a})₂,
(r) —(CH₂)₁₋₃N(R^{a})₂,
(s) —(CH₂)₀₋₃C(═O)R^{t},
(t) —R^{t},
(u) —N(R^{a})R^{t}, and
(v) —(CH₂)₁₋₃R^{t}; or
(6) an 8- to 10-membered heterobicyclic ring containing from 1 to 4 heteroatoms independently selected from oxygen, nitrogen and sulfur, wherein the heterobicyclic ring is saturated or unsaturated, and is unsubstituted or substituted with from 1 to 5 substituents independently selected from:
(a) halogen,
(b) C₁₋₆ alkyl,
(c) —O—C₁₋₆ alkyl,
(d) C₁₋₆ fluoroalkyl,
(e) —O—C₁₋₆ fluoroalkyl,
(f) —CN,
(g) ═O, and
(h) —OH;
R^{t} is naphthyl or a 5- or 6-membered heteromonocylic ring containing from 1 to 4 nitrogen atoms, wherein the heteromonocyclic ring is saturated or unsaturated, and wherein the naphthyl or the heteromonocyclic ring is unsubstituted or substituted with 1 or 2 substituents independently selected from halogen, oxo, C₁₋₄ alkyl, and —O—C₁₋₄ alkyl; and
n is an integer equal to 0, 1 or 2;
and provided that:
(i) when A is phenyl, X is CH, Y is CH, Z¹ is CH, and Q⁴ is —H, then at least one of R¹, R², R³, and R⁴ is not —H;
(ii) when A is phenyl, X is CH, Y is CQ² wherein Q² is halo or —C₁₋₆ alkyl or phenyl optionally substituted with halo or —C₁₋₆ alkyl or benzyl optionally substituted with halo or —C₁₋₆ alkyl, Z¹ is CH, Q⁴ is —H, and all but one of R¹, R², R³ and R⁴ are independently —H, halo or —C₁₋₆ alkyl, then the other of R¹, R², R³ and R⁴ is not —H, halo or —C₁₋₆ alkyl;
(iii) when A is phenyl, X is CH, Y is CH, Z¹ is CH, Q⁴ is —H, and one of R¹, R², R³, and R⁴ is —CO₂R^{a}, then at least one of the others of R¹, R², R³, and R⁴ is not —H;
(iv) when A is phenyl, X is N, Y is C—OH, Z¹ is CH, and Q⁴ is —H, then at least one of R¹, R², R³, and R⁴ is not —H; and
(v) when A is phenyl, X is CH, Y is CH, Z¹ is CQ³, and Q⁴ is —H, then either (v-a) Q³ is not unsubstituted or substituted benzyl or (v-b) at least one of R¹, R², R³, and R⁴ is not —H;
or a pharmaceutically acceptable salt thereof.

5. The compound according to claim 1, which is a compound of Formula (III): 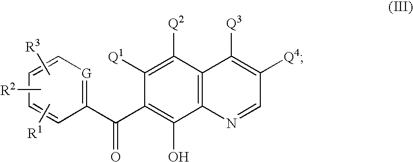
wherein G is N or is CH optionally substituted with one of R¹, R², and R³;
and provided that:
(i) when G is not N and Q¹=Q²=Q³=Q⁴=H, then at least one of R¹, R² and R³ is not —H;
(ii) when G is not N, Q¹ is H, Q² is halo or —C₁₋₆ alkyl or phenyl optionally substituted with halo or —C₁₋₆ alkyl or benzyl optionally substituted with halo or —C₁₋₆ alkyl, Q³=Q⁴=H, and all but one of R¹, R²,and R³ are independently —H, halo or —C₁₋₆ alkyl, then the other of R¹, R², and R³ is not —H, halo or —C₁₋₆ alkyl;
(iii) when G is not N, Q¹=Q²=Q³=Q⁴=H, and one of R¹, R², and R³ is —CO₂R^{a}, then at least one of the others of R¹, R² and R³ is not —H; and
(iv) when G is not N and Q¹=Q²=Q⁴=H, then either (v-a) Q³ is not unsubstituted or substituted benzyl or (v-b) at least one of R¹, R² and R³ is not —H;
or a pharmaceutically acceptable salt thereof.

6. The compound according to claim 1, which is a compound of Formula (V): 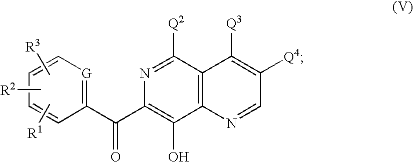
wherein G is N or is CH optionally substituted with one of R¹, R², and R³;
and provided that when G is not N, Q² is OH, and Q³=Q⁴=H, then at least one of R¹, R², and R³ is not —H;
or a pharmaceutically acceptable salt thereof.

7. The compound according to claim 6, wherein
R¹ is:
(1) —R^{k},
(2) —(CH₂)₁₋₄R^{k},
(3) —O—R^{k}, or
(4) —O—(CH₂)₁₋₄R^{k};
R² is:
(1) —H,
(2) methyl,
(3) ethyl,
(4) CF₃,
(5) methoxy,
(6) ethoxy
(7) —OCF₃
(8) halo selected from —F, —Cl and —Br,
(9) —CN,
(10) —CH₂OR^{a},
(11) —CO₂R^{a},
(12) —SR^{a},
(13) —N(R^{a})₂,
(14) —(CH₂)₁₋₃N(R^{a})₂,
(15) —SO₂R^{a},
(16) —(CH₂)_{1- 2}N(R^{a})—C(R^{a})═O,
(17) —R^{k},
(18) —(CH₂)₁₋₄R^{k},
(19) —O—R^{k}, or
(20) —O—(CH₂)₁₋₄R^{k},
each R^{c} is independently —H or —C₁₋₄ alkyl;
each R^{k} is independently:
(1) phenyl which is unsubstituted or substituted with from 1 to 4 substituents independently selected from:
(a) halogen selected from —F, —Cl, and —Br,
(b) methyl,
(c) —CF₃,
(d) methoxy,
(e) —OCF₃,
(f) phenyl,
(g) —S—CH₃,
(h) —CN,
(i) —OH,
(j) phenyloxy, unsubstituted or substituted with from 1 to 3 substituents independently selected from:
(i) halogen selected from —F, —Cl, and —Br,
(ii) methyl,
(iii) —CF₃, and
(iv) —OH,
(k) —N(R^{a})₂,
(I) —(CH₂)₁₋₃N(R^{a})₂,
(m) —R^{t},
(p) —(CH₂)₀₋₃C(═O)N(R^{a})₂, and
(q) —(CH₂)₀₋₃C(═O)R^{a};
(2) —C₃₋₆ cycloalkyl, unsubstituted or substituted with from 1 to 3 substituents independently selected from:
(a) halogen selected from —F, —Cl, and —Br,
(b) methyl,
(c) —CF₃,
(d) methoxy,
(e) —OCF₃,
(f) —CN,
(h) phenyl, and
(j) —OH;
(3) a 5- or 6- membered heteroaromatic ring selected from thienyl, pyridyl, imidazolyl, pyrrolyl, pyrazolyl, thiazolyl, isothiazolyl, oxazolyl, isooxazolyl, pyrazinyl, pyirimidinyl, triazolyl, tetrazolyl, furanyl, and pyridazinyl, wherein the heteroaromatic ring is unsubstituted or substituted on nitrogen or carbon with 1 or 2 substituents independently selected from:
(a) halogen selected from —F, —Cl, and —Br,
(b) methyl,
(c) —CF₃,
(d) methoxy,
(e) —OCF₃,
(f) phenyl,
(g) —S—C₁₋₆ alkyl,
(h) —CN,
(i) —OH,
(j) phenyloxy, unsubstituted or substituted with from 1 to 3 substituents independently selected from:
(i) halogen selected from —F, —Cl, and —Br,
(ii) methyl,
(iii) —CF₃, and
(iv) —OH,
(k) —N(R^{a})₂,
(l) —C₁₋₆ alkyl-N(R^{a})₂,
(m) —R^{t},
(n) oxo,
(o) —(CH₂)₀₋₃C(═O)N(R^{a})₂, and
(p) —(CH₂)₀₋₃C(═O)R^{a}; and
(4) a 5- or 6- membered saturated heterocyclic ring selected from piperidinyl, morpholinyl, thiomorpholinyl, thiazolidinyl, isothiazolidinyl, oxazolidinyl, isooxazolidinyl, pyrrolidinyl, imidazolidinyl, piperazinyl, tetrahydrofuranyl, and pyrazolidinyl, wherein the heterocyclic ring is unsubstituted or substituted with 1 or 2 substituents independently selected from:
(a) halogen selected from —F, —Cl, and —Br,
(b) methyl,
(c) —CF₃,
(d) methoxy,
(e) —OCF₃,
(f) —CN,
(g) ═O,
(h) phenyl,
(i) benzyl,
(j) phenylethyl,
(k) —OH,
(l) —(CH₂)₀₋₃C(═O)N(R^{a})₂,
(m) —(CH₂)₀₋₃C(═O)R^{a},
(n) N(R^{a})—C(═O)R^{a},
(o) N(R^{a})—C(═O)OR^{a},
(p) N(R^{a})—C(═O)OC(CH₃)₃,
(q) (CH₂)₁₋₃N(R^{a})—C(═O)R^{a},
(r) N(R^{a})₂,
(s) (CH₂)₁₋₃N(R^{a})₂,
(t) —(CH₂)₀₋₃C(═O)R^{t},
(u) —R^{t},
(v) —N(R^{a})R^{t}, and
(w) —(CH₂)₁₋₃R^{t}; and
R^{t} is selected from pyrrolidinyl, pyrazolidinyl, imidazolinyl, piperidinyl, piperazinyl, pyrrolyl, pyridyl, imidazolyl, pyrazolyl, triazolyl, tetrazolyl, pyrazinyl, pyrimidinyl, and pyradizinyl; any one of which is unsubstituted or substituted with 1 or 2 substituents independently selected from —F, —Cl, —Br, oxo, methyl, and methoxy;
or a pharmaceutically acceptable salt thereof.

8. The compound according to claim 1, which is a compound selected from the group consisting of:
1-(3-Benzylphenyl)-1-(8-hydroxyquinolin-7-yl)methanone;
1-(3-Benzylphenyl)-1-(8-hydroxy-4-methylquinolin-7-yl)methanone;
1-(3-Benzylphenyl)-1-(8-hydroxy-5-methylquinolin-7-yl)methanone;
1-[3-Benzyl-5-(1H-1,2,4-triazol-1-ylmethyl)phenyl]-1-(5-chloro-8-hydroxyquinolin-7-yl)methanone;
1-(3-Benzyl-5-imidazol-1-ylmethylphenyl)- 1-(5-chloro-8-hydroxyquinolin-7-yl)methanone;
1-(4-Benzyl-pyridin-2-yl)-1-(8-hydroxyquinolin-7-yl)methanone;
1-(3-Benzylphenyl)-1-(8-hydroxy-[1,6]naphthyridin-7-yl)methanone;
1-[3-Benzyl-5-(1,1-dioxoisothiazolidin-2-ylmethyl)-phenyl]-1-(8-hydroxy-[1,6]naphthyridin-7-yl)methanone;
1-(3-Benzyl-5-(morpholin-4-ylmethyl)phenyl)-1-(8-hydroxy-[1,6]naphthyridin-7-yl)methanone;
1-(3-Benzyl-5-piperidin-1-ylmethylphenyl)-1-(8-hydroxy-[1,6]naphthyridin-7-yl)methanone;
1-[3-Benzyl-5-(4-methylpiperazin-1-ylmethyl)phenyl]-1-(8-hydroxy-[1,6]naphthyridin-7-yl)methanone;
1-{3-Benzyl-5-[1-(8-hydroxy-[1,6]naphthyridin-7-yl)methanoyl]benzyl}-1H-pyridin-2-one;
3-{3-Benzyl-5-[(8-hydroxy-1,6-naphthyridin-7-yl)carbonyl]benzyl}-1-methylpyrimidine-2,4-(1H,3H)-dione;
1-[3-Benzyl-5-(tetrazol-1-ylmethyl)phenyl]-1-(8-hydroxy-[1,6]naphthyridin-7-yl)methanone;
1-[3-Benzyl-5-(tetrazol-2-ylmethyl)phenyl]-1-(8-hydroxy-[1,6]naphthyridin-7-yl)methanone;
1-(3-Benzyl-5-pyrazol-1 -ylmethylphenyl)-1-(8-hydroxy- [1,6]naphthyridin-7-yl)methanone;
3-{3-Benzyl-5-[1-(8-hydroxy-[1,6]naphthyridin-7-yl)methanoyl]benzyl}-3H-pyrimidin-4-one;
1- {3-Benzyl-5-[1-(8-hydroxy-[1,6]naphthyridin-7-yl)methanoyl]benzyl}pyrrolidin-2-one;
N-{3-Benzyl-5-[1-(8-hydroxy-[1,6]naphthyridin-7-yl)methanoyl]benzyl}formamide;
N-{3-Benzyl-5-[1-(8-hydroxy-[1,6]naphthyridin-7-yl)methanoyl]benzyl}—N-methylformamide;
1-(8-hydroxy-[1,6]naphthyridin-7-yl)-1-(3-pyrazol-1-ylmethyl-5-pyridin-2-ylmethylphenyl)methanone;
1-(8—Hydroxy-[1,6]naphthyridin-7-yl)-1-[3-(1,1-dioxo-isothiazolidin-2-ylmethyl)-5-pyridin-2-ylmethylphenyl]methanone;
1-(8—Hydroxy-[1,6]naphthyridin-7-yl)-1-[3-(pyridin-2-one-1-ylmethyl)-5-pyridin-2-ylmethylphenyl]methanone;
1-(8—Hydroxy-[1,6]naphthyridin-7-yl)-1-[3-(piperidin-2-one-1-ylmethyl)-5-pyridin-2-ylmethylphenyl]methanone;
7-[1 -(4-Benzylpyridin-2-yl)methanoyl]-8-hydroxy-6H-[1,6]naphthyridin-5-one;
and pharmaceutically acceptable salts thereof.

9. A pharmaceutical composition comprising a therapeutically effective amount of a compound according to claim 1 and a pharmaceutically acceptable carrier.

10. A pharmaceutical composition which comprises the product made by combining a therapeutically effective amount of a compound according to claim 1 and a pharmaceutically acceptable carrier.

11. A method of inhibiting HIV integrase, preventing or treating infection by HIV, or treating or delaying the onset of AIDS in a subject in need thereof which comprises administering to the subject a therapeutically effective amount of a compound A compound of Formula (I): 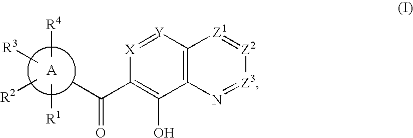
or a pharmaceutically acceptable salt thereof;
wherein A is
(1) phenyl,
(2) phenyl fused to a carbocycle to form a fused carbocyclic ring system; or
(3) heterocycle containing one or more heteroatoms selected from nitrogen, oxygen and sulfur and a balance of carbon atoms, with at least one of the ring atoms being carbon;
A is connected by a ring carbon to the exocyclic carbonyl, and is substituted by R¹, R², R³, and R⁴;
X is N or C-Q¹;
Z¹ is N or C-Q³;
Z² is N or C-Q⁴;
Z³ is N or CH;
each of Q¹, Q², Q³, and Q⁴ is independently
(1) —H,
(2) —C₁₋₆ alkyl,
(3) —C₁₋₆ fluoroalkyl,
(4) —OH,
(5) —O—C₁₋₆ alkyl,
(6) —O—C₁₋₆ fluoroalkyl,
(7) halo,
(8) —CN,
(9) —C₁₋₆ alkyl-OR^{a},
(10) —C₀₋₆ alkyl-C(═O)R^{a},
(11) —C₀₋₆ alkyl-CO₂R^{a},
(12) —C₀₋₆ alkyl-SR^{a},
(13) —N(R^{a})₂,
(14) —C₁₋₆ alkyl —N(R^{a})₂,
(15) —C₀₋₆ alkyl-C(═O)N(R^{a})₂,
(16) —C₁₋₆ alkyl-N(R^{a})—C(R^{a})═O,
(17) —SO₂R^{a},
(18) —N(R^{a})SO₂R^{a},
(19) —C₂₋₅ alkynyl,
(20) —C₂₋₅ alkynyl-CH₂N(R^{a})₂,
(21) —C₂₋₅ alkynyl-CH₂OR^{a},
(22) 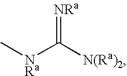
(23) —N(R^{a})—C₁₋₆ alkyl-SR^{a},
(24) —N(R^{a})—C₁₋₆ alkyl-OR^{a},
(25) —N(R^{a})—C₁₋₆ alkyl-N(R^{a})₂,
(26) —N(R^{a})—C₁₋₆ alkyl-N(R^{a})—C(R^{a})═O,
(27) —R^{k},
(28) —C₁₋₆ alkyl substituted with R^{k},
(29) —C₁₋₆ fluoroalkyl substituted with R^{k},
(30) —C₂₋₅ alkenyl-R^{k},
(31) —C₂₋₅ alkynyl-R^{k},
(32) —O—R^{k},
(33) —O—C₁₋₄ alkyl-R^{k},
(34) —S(O)ₙ—R^{k},
(35) —S(O)ₙ—C₁₋₄ alkyl-R^{k},
(36) —O—C₁₋₆ alkyl-OR^{k},
(37) —O—C₁₋₆ alkyl-O—C₁₋₄ alkyl-R^{k},
(38) —O—C₁₋₆ alkyl-SR^{k},
(39) —N(R^{c})—R^{k},
(40) —N(R^{c})-C₁₋₆ alkyl substituted with one or two R^{k} groups;
(41) —N(R^{c})—C₁₋₆ alkyl-OR^{k},
(42) —C(═O)N—C₁₋₆ alkyl-R^{k}, or
(43) —C₂₋₅ alkynyl-CH₂S(O)ₙ-R^{a};
each of R¹ and R² is independently:
(1) —H,
(2) —C₁₋₆ alkyl,
(3) —C₁₋₆ fluoroalkyl,
(4) —O—C₁₋₆ alkyl,
(5) —O—C₁₋₆ fluoroalkyl,
(6) —OH,
(7) halo,
(8) —NO₂,
(9) —CN,
(10) —C₁₋₆ alkyl-OR^{a},
(11) —C₀₋₆ alkyl-C(═O)R^{a},
(12) —C₀₋₆ alkylCO₂R^{a},
(13) —C₀₋₆ alkyl-SR^{a},
(14) —N(R^{a})₂,
(15) —C₁₋₆ alkyl-N(R^{a})₂,
(16) —C₀₋₆ alkyl-C(═O)N(R^{a})₂,
(17) —C₁₋₆ alkyl-N(R^{a})—C(R^{a})═O,
(18) —SO₂R^{a},
(19) —N(R^{a})SO₂R^{a},
(20) —C₂₋₅ alkenyl,
(21) —O—C₁₋₆ alkyl-OR^{a},
(22) —O—C₁₋₆ alkyl-SR^{a},
(23) —O—C₁₋₆ alkyl-NH—CO₂R^{a},
(24) —O—C₂₋₆ alkyl-N(R^{a})₂,
(25) —N(R^{a})—C₁₋₆ alkyl-SR^{a},
(26) —N(R^{a})—C₁₋₆ alkyl-OR^{a},
(27) —N(R^{a})—C₁₋₆ alkyl-N(R^{a})₂,
(28) —N(R^{a})—C₁₋₆ alkyl-N(R^{a})—C(R^{a})═O,
(29) —R^{k},
(30) —C₁₋₆ alkyl substituted with 1 or 2 R^{k} groups,
(31) —C₁₋₆ fluoroalkyl substituted with 1 or 2 R^{k} groups,
(32) —C₂₋₅ alkenyl-R^{k},
(33) —C₂₋₅ alkynyl-R^{k},
(34) —O—R^{k},
(35) —O—C₁₋₄ alkyl-R^{k},
(36) —S(O)ₙ—R^{k},
(37) —S(O)ₙ—C₁₋₄ alkyl-R^{k},
(38) —O—C₁₋₆ alkyl-OR^{k},
(39) —O—C₁₋₆ alkyl-O—C₁₋₄ alkyl-R^{k},
(40) —O—C₁₋₆ alkyl-SR^{k},
(41) —C₁₋₆ alkyl (OR^{b})(R^{k}),
(42) —C₁₋₆ alkyl (OR^{b})(—C₁₋₄ alkyl-R^{k}),
(43) —C₀₋₆ alkyl-N(R^{b})(R^{k}),
(44) —C₀₋₆ alkyl-N(R^{b})(—C₁₋₄ alkyl-R^{k}),
(45) —C₁₋₆ alkyl S(O)ⁿ—R^{k},
(46) —C₁₋₆ alkyl S(O)ₙ—C₁₋₄ alkyl-R^{k},
(47) —C₀₋₆ alkyl C(O)—R^{k}, or
(48) —C₀₋₆ alkyl C(O)—C₁₋₄ alkyl-R^{k};
each of R³ and R⁴ is independently
(1) —H,
(2) halo,
(3) —CN,
(4) —NO₂,
(5) —OH,
(6) C₁₋₆ alkyl,
(7) C₁₋₆ fluoroalkyl,
(8) —O—C₁₋₆ alkyl,
(9) —O—C₁₋₆ fluoroalkyl,
(10) —C₁₋₆ alkyl-OR^{a},
(11) —C₀₋₆ alkyl-C(═O)R^{a},
(12) —C₀₋₆ alkyl-CO₂R^{a},
(13) —C₀₋₆ alkyl-SR^{a},
(14) —N(R^{a})₂,
(15) —C₁₋₆ alkyl-N(R^{a})₂,
(16) —C₀₋₆ alkyl-C(═O)N(R^{a})₂,
(17) —SO₂R^{a},
(18) —N(R^{a})SO₂R^{a},
(19) —C₂₋₅ alkenyl,
(20) —O—C₁₋₆ alkyl-OR^{a},
(21) —O—C₁₋₆ alkyl-SR^{a},
(22) —O—C₁₋₆ alkyl-NH—CO₂R^{a},
(23) —O—C₂₋₆ alkyl-N(R^{a})₂, or
(24) oxo;
each R^{a} is independently —H, —C₁₋₆ alkyl, or —C16 fluoroalkyl;
each R^{b} is independently:
(1) —H,
(2) —C₁₋₄ alkyl,
(3) —C₁₋₄ fluoroalkyl,
(4) —R^{k},
(5) —C₂₋₃ alkenyl,
(6) —C₁₋₄ alkyl-R^{k},
(7) —C₂₋₃ alkenyl-R^{k},
(8) —S(O)ₙ—R^{k}, or
(9) —C(O)—R^{k};
each R^{c} is independently
(1) —H,
(2) —C₁₋₆ alkyl,
(3) —C₁₋₆ alkyl substituted with —N(R^{a})₂, or
(4) —C₁₋₄ alkyl-aryl, wherein aryl is optionally substituted with 1 to 5 substituents independently selected from halogen, C₁₋₆ alkyl, C₁₋₆ fluoroalkyl, —O—C₁₋₆ alkyl, —O—C₁₋₆ fluoroalkyl, —S-C₁₋₆ alkyl, —CN, and —OH;
each R^{k} is independently carbocycle or heterocycle, wherein the carbocycle and heterocycle are unsubstituted or substituted with from 1 to 5 substituents each of which is independently selected from
(a) halogen,
(b) C₁₋₆ alkyl,
(c) C₁₋₆ fluoroalkyl,
(d) —O—C₁₋₆ alkyl,
(e) —O—C₁₋₆ fluoroalkyl,
(f) —S—C₁₋₆ alkyl,
(g) —CN,
(h) —OH,
(i) oxo,
(j) —(CH₂)₀₋₃C(═O)N(R^{a})₂,
(k) —(CH₂)₀₋₃C(═O)R^{a},
(l) —N(R^{a})—C(═O)R^{a},
(m) —N(R^{a})—C(═O)OR^{a},
(n) —(CH₂)₁₋₃N(R^{a})—C(═O)R^{a},
(o) —N(R^{a})₂,
(p) —C₁₋₆ alkyl-N(R^{a})₂,
(q) aryl,
(r) aryloxy-,
(s) —C₁₋₄ alkyl substituted with aryl,
(t) heteromonocycle,
(u) —C₁₋₄ alkyl substituted with a heteromonocycle,
(v) heteromonocyclylcarbonyl-C₀₋₆ alkyl-,
(w) N-heteromonocyclyl-N-C₁₋₆ alkyl-amino-;
wherein the aryl group in (q) aryl, (r) aryloxy, and (s) —C₁₋₄ alkyl substituted with aryl, is optionally substituted with from 1 to 3 substituents independently selected from halogen, C₁₋₆ alkyl, —O—C₁₋₆ alkyl, C₁₋₆ alkyl substituted with N(R^{a})₂, C₁₋₆ fluoroalkyl, and —OH; and
wherein the heteromonocyclyl group in (t) heteromonocycle, (u) —C₁₋₄ alkyl substituted with a heteromonocycle, (v) heteromonocyclyl-carbonyl-C₀₋₆ alkyl-, and (w) N-heteromonocyclyl-N-C₁₋₆ alkyl-amino- is optionally substituted with from 1 to 3 substituents independently selected from halogen, C₁₋₆ alkyl, —O—C₁₋₆ alkyl, C₁₋₆ fluoroalkyl, oxo, and —OH; and
each n is independently an integer equal to 0, 1 or 2.


SEQUENCE LISTING (2 sequences)

SEQ ID NO: 1 - DNA, 20 bp, Artificial Sequence
  Synthetic DNA
tgaccaagggctaattcact

SEQ ID NO: 2 - DNA, 20 bp, Artificial Sequence
  Synthetic DNA
actggttcccgattaagtga